(19) United States
(12) Patent Application Publication
(10) Pub. No.: US 2026/0265804 A1
(43) Pub. Date: Sep. 10, 2026

(54) REDOX ACTIVE ANTIFOULING COATINGS

(71) Applicant: PRESIDENT AND FELLOWS OF HARVARD COLLEGE, Cambridge, MA (US)

(72) Inventors: Sajjad Janfaza, Cambridge, MA (US); Pawan Jolly, Cambridge, MA (US); Nandhinee Radha Shanmugam, Cambridge, MA (US); Donald E. Ingber, Cambridge, MA (US)

(73) Assignee: PRESIDENT AND FELLOWS OF HARVARD COLLEGE, Cambridge, MA (US)

(21) Appl. No.: 19/167,152

(22) PCT Filed: Mar. 22, 2024
(86) PCT No.: PCT/US2024/021067

§ 371 Date: Sep. 19, 2025

Related U.S. Application Data
(60) Provisional application No. 63/453,931, filed on Mar. 22, 2023.

(51) Int. Cl. C12Q 1/00; C09D 5/14; C09D 5/16; C09D 5/24; C09D 7/61; C09D 7/62; C09D 7/63; C09D 189/00; G01N 27/327

(52) U.S. Cl. CPC C12Q 1/006; C09D 5/14; C09D 5/1625; C09D 5/1656; C09D 5/24; C09D 7/61; C09D 7/62; C09D 7/63; C09D 189/00; G01N 27/3271

(57) ABSTRACT

The disclosure relates generally to compositions and methods for making antifouling and electrically responsive coatings, and electrodes and sensors including said antifouling coatings, and uses thereof.

### CROSS-REFERENCE TO RELATED APPLICATIONS

This application claims benefit under 35 U.S.C. § 119(e) of U.S. Provisional Application No. 63/453,931, filed Mar. 22, 2023, contents of which are incorporated herein by reference in their entirety.

### GOVERNMENT SUPPORT

This invention was made with government support under 75A50121C00075 awarded by Biomedical Advanced Research and Development Authority (BARDA). The government has certain rights in this invention.

### TECHNICAL FIELD

The disclosure relates generally to compositions and methods for making antifouling and electrically responsive coatings and electrodes and organic sensors including said coatings, and uses thereof.

### BACKGROUND

An imbalance of metabolites is considered one of the most severe threats to human health and is related to the high risks of several serious illnesses. The increasing growth of metabolomic analyses over the last decade shows the trend for developing new diagnostic methods based on detection of changes in levels of metabolites that are indicative of various disease states (e.g., diabetes, cancer, etc.). Therefore, fast and reliable detection of these biomarkers at low cost can be of great value for disease diagnosis in hospitals and at the point-of-care (POC).

Metabolite concentrations can be influenced by factors of intrinsic nature (biochemical reactions) or exogenous origin (diet or medication). Thus, continuous measurement of metabolite levels in biological samples, such as blood, saliva, and urine, is of central importance in clinical diagnostics. However, real-time measurement of metabolite concentrations in complex biological fluids presents considerable challenges because non-specific binding of other materials in these fluids can decrease sign specificity and sensitivity over time.

Thus, there is a need for electrical transducers and methods that can prevent non-specific interactions and preserve the ability of the electrical transducer to record electrochemical signals with high sensitivity. The present disclosure addresses these needs.

### SUMMARY

In general, various aspects described herein relate to compositions and their application to surfaces (e.g., conducting and/or transducer surfaces). The coatings protect these surfaces from unwanted interactions that impede or diminish their intended function. Furthermore, the compositions described herein allow one to produce coatings with high sensitivity and antifouling activity.

In one aspect provided herein is a composition, e.g., a mixture, such as homogenous mixture. The composition comprises: a proteinaceous material; a conductive element; a redox mediator, optionally the redox mediator is covalently linked to the proteinaceous material or the conductive element; and an oxidoreductase, optionally, the oxidoreductase is covalently linked to the proteinaceous material or the conductive element.

The compositions described herein can be used to coat surfaces to provide antifouling coating layer on the surface. Accordingly, in another aspect, provided herein is a surface comprising an antifouling coating layer on at least a portion of the surface, wherein the composition comprises: a proteinaceous material; a conductive element; a redox mediator, optionally the redox mediator is covalently linked to the proteinaceous material or the conductive element; and an oxidoreductase, optionally, the oxidoreductase is covalently linked to the proteinaceous material or the conductive element.

In some embodiments of any one of the aspects described herein, the antifouling coating layer is directly or indirectly connected with an electrode. For example, the surface is a surface of a conductive substrate (e.g., an electrically conductive substrate). Accordingly, in some embodiments, the substrate is an electrode. In some embodiments of any one of the aspects described herein, the surface is a surface of a medical device.

Accordingly, in another aspect provided herein is an electrode. The electrode comprises: (i) a conductive substrate (e.g., an electrically conductive substrate); and (2) an antifouling coating layer on at least a portion of a surface of the conductive substrate, and wherein the antifouling coating layer comprises: a proteinaceous material; a conductive element; a redox mediator, optionally the redox mediator is covalently linked to the proteinaceous material, the conductive element or the conductive surface; and an oxidoreductase, optionally, the oxidoreductase is covalently linked to the proteinaceous material, the conductive element or the conductive surface.

Exemplary redox mediators include, but are not limited to, metallocenes, metallocence derivatives, quinones, polycyclic aromatic hydrocarbons, redox-active organic molecules, phenazines, organosulfur compounds, radical acceptors, or a combination thereof. In some embodiments, the redox mediator is ferrocene or a derivative of ferrocene. For example, the redox mediator is aminoferrocene.

Exemplary oxidoreductases include, but are not limited to, oxidases, reductases, dehydrogenases, peroxidases, oxygenases, monooxygenases, dioxygenases, lipoxygenases, hydrogenases, transhydrogenases, peroxidases, catalases, epoxidases, hydroxylases, demethylases, desaturases, dismutases, hydroxyltransferases, synthases, dehalogenases, and deiodinases. In some embodiments of any one of the aspects described herein, the oxidoreductase is an oxidase or a dehydrogenase. For example, oxidoreductase is glucose oxidase.

It is noted that the redox mediator can be present in the antifouling coating layer or at a surface of the antifouling coating layer. Accordingly, in some embodiments of any one of the aspects described herein, the redox mediator is on a surface of the antifouling coating layer. In some embodiments of any one of the aspects described herein, the redox mediator is embedded within the antifouling coating layer. In some embodiments of any one of the aspects described herein, the redox mediator is imprinted on the antifouling coating layer. In some embodiments of any one of the aspects described herein, the redox mediator is in pores of the antifouling coating layer.

Similar to the redox mediator, the oxidoreductase can be present in the antifouling coating layer or at a surface of the antifouling coating layer. Accordingly, in some embodiments of any one of the aspects described herein, the oxidoreductase is on a surface of the antifouling coating layer. In some embodiments of any one of the aspects described herein, the oxidoreductase is embedded within the antifouling coating layer. In some embodiments of any one of the aspects described herein, the oxidoreductase is imprinted on the antifouling coating layer. In some embodiments of any one of the aspects described herein, the oxidoreductase is in pores of the antifouling coating layer.

In some embodiments of any one of the aspects described herein, the components of the antifouling coating layer are present in separate layers. For example, the antifouling coating layer has layered structure. In some embodiments, the antifouling coating layer comprises a first layer and a second layer, where the first layer comprises the protienaceous material and the conductive element, and the second layer comprises the redox mediator and the oxidoreductase. Generally, the first layer, i.e., the layer comprising the protienaceous material and the conductive element is in direct contact with the surface of the substrate and the second layer comprising the redox mediator and the oxidoreductase is on a surface of the first layer opposite the substrate surface.

In some embodiments, the antifouling coating layer comprises a first layer, a second layer, and a third layer. The second layer is disposed between the first and third layers. The first layer comprises the protienaceous material and the conductive element. The second layer comprises the redox mediator. The third layer comprises the oxidoreductase. The first layer, i.e., the layer comprising the protienaceous material and the conductive element is in direct contact with the surface of the substrate. The second layer comprising the redox mediator is on a surface of the first layer opposite the substrate surface. Third layer comprising oxidoreductase is on a surface of the second layer opposite the first layer.

In some embodiment of any one of the aspects described herein, the composition and/or the antifouling coating layer described herein further comprises a target binding molecule. Exemplary target binding molecules include, but are not limited to, receptors, ligand for a receptor, antibodies, antigen binding fragment of an antibody, antigens, enzymes, and nucleic acids. In some embodiments, the target binding molecule is an antibody or antigen binding fragment of an antibody.

In some embodiments of any one of the aspects described herein, the composition and/or the antifouling coating layer described herein further comprises a polymer. For example, the antifouling coating layer or the composition comprises a water miscible polymer. In some embodiments of any one of the aspects described herein, the composition and/or the antifouling coating layer described herein comprises a degradable polymer. Some exemplary polymers include, but are not limited to, poly(N-isopropyl acrylamide) (PNIPAAm), polyethylene glycol (PEG), alginate, polytetrafluoroethylene (PTFE), polyacetylene (PA), polyaniline (PANI), polypyrrole (PPy), polythiophene (PTH), poly(para-phenylene) (PPP), poly(phenylenevinylene) (PPV), and polyfuran (PF).

In some embodiments of any one of the aspects described herein, the composition and/or the antifouling coating layer described herein comprises a polymer selected from the group consisting of poly(3-hexylthiophene) (P3HT), perfluoro-3,6-dioxa-4-methyl-7-octenesulfonic acid copolymer, tetrafluoroethylene-perfluoro-3,6-dioxa-4-methyl-7-octenesulfonic acid copolymer (e.g. nafion), poly(9,9-dioctylfluorene-2,7-diyl-co-bis-N,N-(4-butylphenyl)-bis-N,N-phenyl-1,4-phenylenediamine), poly(9,9-dioctylfluorene-2,7-diyl-co-benzothiadiazole), (6,6)-phenyl-C61-butyric acid methyl ester, poly(2-methoxy-5-(2′-ethyl-hexyloxy)-1,4-phenylene vinylene), poly(4-vinylphenol) (PVP), and copolymers thereof, and any combination thereof.

In some embodiments of any one of the aspects described herein, the antifouling coating layer has a porosity of about 5% to about 95%. For example, the antifouling coating layer has a porosity of about 20% to about 75%. In some embodiments, the antifouling coating layer has a porosity of about 25% to about 60%, or about 30% to about 50%. For example, the antifouling coating layer has a porosity of about 35% to 45%.

In some embodiments of any one of the aspects described herein, the antifouling coating layer comprises macropores. In some embodiments of any one of the aspects described herein, the antifouling coating layer comprises mesopores. In some embodiments of any one of the aspects described herein, the antifouling coating layer comprises nanopores. In some embodiments of any one of the aspects described herein, the antifouling coating layer comprises multiscale pores, i.e., both macropores and mesopores.

In still another aspect, provided herein is a sensor comprising an electrode or a coated surface described herein. Generally, the sensor comprises a fluid-contact surface and the electrode is immobilized on at least a portion of the fluid-contact surface. In some embodiments of any one of the aspects described herein, the fluid-contact surface further comprises a positive control electrode and/or a negative control electrode immobilized thereon. In some embodiments of any one of the aspects described herein, the sensor comprises one or more microfluidic flow cells.

The electrodes, surfaces and sensors described herein are useful for detecting a target analyte in a sample. Accordingly, in yet another aspect, provided herein is a use of a surface, an electrode or sensor described herein for detecting a target analyte in a sample.

In still another aspect, provided herein is a method for detecting a target analyte in a sample. Generally, the method comprises contacting a sample suspected of comprising a target analyte with an electrode described herein and detecting the oxidation or reduction of the target analyte by the oxidoreductase present in or on the antifouling coating. In some embodiments, said detecting comprising and detecting the oxidation or reduction of the redox mediator present in or on the antifouling coating.

In some embodiments of any one of the aspects described herein, the target analyte is a peptide, a polypeptide, a peptidomimetic, a nucleic acid, an oligosaccharide, a polysaccharide, an amino acid, nucleoside, a nucleotide, a carbohydrate, a lipid, a peptidoglycan, a cell, microbial matter, an antigen, a lipid, a steroid, a hormone, a lipopolysaccharide, an endotoxin, a therapeutic agent, a lipid-binding molecule, a cofactor, a small molecule, a toxin, a biological threat agent (e.g., spore, viral, cellular and protein toxin), or any combination thereof.

The sample suspected of comprising the target analyte can be a biological sample (e.g., blood, saliva, amniotic fluid, sputum, urine, semen, cerebrospinal fluid, bronchial aspirate, perspiration, mucus, liquefied stool sample, synovial fluid, lymphatic fluid, tears, tracheal aspirate, lactation product, and any combination thereof); or a food, an ingredient for preparing a food, poultry, meat, fish, beverage, or dairy product; or a non-biological sample (e.g., water, salt water, pond water, river water, reservoir water, brine, drinking water, industrial water, brown water, waste water, sewerage, soil, and mixtures thereof.

In another aspect, provided herein is a kit comprising a composition, surface, electrode, or sensor described herein.

### BRIEF DESCRIPTION OF THE DRAWINGS

This patent or application file contains at least one drawing executed in color. Copies of this patent or patent application publication with color drawings will be provided by the Office upon request and payment of the necessary fee.

FIG. 1 is a schematic representation of structure of an exemplary working electrode for an electrochemical biosensor.

FIG. 2 shows electrochemical CV measurements of the antifouling coating containing redox molecule in PBS in the absence and presence of glucose at scan rates of 200 mV/s.

FIGS. 3A-3B show chronoamperometry of the biosensor in different concentrations of glucose in PBS (FIG. 3A) and a calibration curve of the biosensor (FIG. 3B).

FIGS. 4A-4B show the maximum current (FIG. 4A) and chronoamperometry (FIG. 4B) of the biosensor in 5 mM Glu in PBS, BSA, Uric acid, and Dopamine.

FIG. 5 shows the response of the biosensor to Glucose and Sucrose.

FIG. 6 shows the response of the biosensor to Glucose and Fructose.

FIG. 7 shows the response of biosensor to presence and absence of plasma (0.033, 0.072, 0.361, 1.953 mgmL⁻¹) and saliva (less than 0.02 and 1.16 mgmL⁻¹).

FIG. 8 shows schematic diagram representing the steps involved in the fabrication of glucose biosensors. The sensor is constructed sequentially with a nanocomposite-based antifouling coating, surface functionalized redox, and enzyme layers that support glucose detection in biological samples.

FIGS. 9A-9D show the Electrochemical performance of NH₂-Fc functionalized prGOx/BSA/GA in PBS. (FIG. 9A) Comparison of CV response for bare and NH₂-Fc functionalized nanocomposite coated SPCE. (FIG. 9B) CV of NH₂-Fc functionalized SPCE measured in PBS at different scan rates. (FIG. 9C) Plot demonstrating linear relationship between logarithm of peak current vs. logarithm of scan rate. (FIG. 9D) Cyclic voltammogram recorded in PBS for 20 cycles demonstrating electrochemical stability of NH₂-Fc functionalized prGOx/BSA/GA coating.

FIGS. 10A and 10B show (FIG. 10A) Chronoamperometric response of NH₂-Fc functionalized prGOx/BSA/GA sensor at 0.2 V in PBS obtained with successive addition of glucose from 0 to 50 mM. (FIG. 10B) Calibration dose response curve derived from amperometric signal response. The graph shows linear range between 0 and 10 mM glucose concentrations and saturation for concentrations higher than 20 mM of glucose. Data represented here is from n=2 replicates.

FIGS. 11A-11D show (FIG. 11A) Chronoamperometric response obtained at 0.2 V from NH₂-Fc functionalized glucose biosensor for a 5 mM glucose in PBS spiked with various electroactive interferants, such as BSA, uric acid, and dopamine. (FIG. 11B) Box plot representation of current response obtained with 2.5 mg/mL BSA, 1 mM uric acid, and 10 μM dopamine spiked separately in a solution containing 5 mM glucose in PBS. (FIG. 11C) Amperometry response of NH₂-Fc functionalized glucose sensor with varying concentrations of fructose demonstrating the sensor specificity. (FIG. 11D) Plot showing the specificity of the developed glucose biosensor for glucose detection compared with other saccharides, such as sucrose and fructose. Data represented here is from n=2 replicates.

FIG. 12 shows Response of biosensor to presence and absence of plasma (0.183 mM, 0.399 mM, 0.200 mM, 10.85 mM) and saliva (less than 0.111 mM and 6.44 mM).

FIG. 13 shows the calibration curve of glucose standard solutions using colorimetric assay. Linear regression analysis: y=9.1417x with an R2 of 0.9967, demonstrating a strong correlation between the variables.

### DETAILED DESCRIPTION

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated by reference in their entirety for any purpose

The compositions, electrodes, surfaces, sensors and structures provided herein are based in part on a composition comprising: a proteinaceous material; a conductive element; a redox mediator; and an oxidoreductase. Optionally, the redox mediator and/or the oxidoreductase are independently linked to the proteinaceous material or the conductive element.

### Redox Mediator

Embodiments of the various aspects described herein include a redox mediator. As used herein, term “redox mediator” refers to any chemical moiety capable of undergoing a reduction (accepting of an electron(s)) or oxidation (donation of an electron(s)) in the course of a multi-step process transferring electrons to or from a substrate of an oxidoreductase to an electrode. Exemplary redox mediators include, but are not limited to, metallocenes, metallocence derivatives, quinones, polycyclic aromatic hydrocarbons, redox-active organic molecules, phenazines, organosulfur compounds, radical acceptors, or a combination thereof.

In some embodiments of any one of the aspects described herein, the redox mediator is selected from the group consisting of ferrocene, ferrocene derivatives, 3,3′,5,5′-tetramethylbenzidine (TMB), o-phenylenediamine dihydrochloride (OPD), N,N,N′,N′-tetramethyl-p-phenylenediamine, viologens 2,2′-azino-bis(3-ethylbenzothiazoline-6-sulfonic acid) (ABTS), p-nitrophenyl Phosphate (PNPP), 3,3′-diaminobenzidine (DAB), 4-chloro-1-naphthol (4-CN), 5-bromo-4-chloro-3-indolyl-phosphate (BCIP), nitro blue tetrazolium (NBT), methylene blue, tetrathialfulvaene, 2,6-dichloroindophenol (DCIP), 2,6-dichloroindophenyl phosphate, riboflavin 5′-monophosphate (RMP), ethyl viologen (1,1′-bis(ethyl)-4,4′-bipyridinium or BEBP), Paraquat, hexaamine ruthenium, 1,4-naphthoquione, resorufine, cyanomethylviologen, diquat, triquat, 1,1′-bis(2-sulfoethyl)-4,4′-bipyridinium, 1,1′-dibenzyl-4,4′-bipyridinium, 4,4′-dicarboxy-2,2′-bipyridyl, 1-hydroxybenzotriazole, veratryl alcohol, violuric acid, 2-methoxy-phenothiazone, 3-hydroxyanthranilic acid, anthraquinone 2,6-disulfonic acid, N-hydroxyacetanilide, phenol red, 3,3′,5,5′-tetramethyl benzidine, dichlorophenol red, 2,2′,6,6′-tetramethylpiperidine-N-oxyl radical, syringaldehyde and acetosyringone, 2,2,6,6-tetramethylpiperidin-1-yl)oxyl (TEMPO), 2,2′7,7′-tetrakis-(N,N-di-p-methoxyphenyl-amine)-9,9′-spirobifluorene (spiro-MeOTAD), sodium anthraquinone-2,6-di sulphonate (AQDS), benzoquinones, 2,2′-biimidazole, 2-(2-pyridyl) imidazole, 2,2′-bipyridine, tetracyanoquinodimethane (TCNQ), and combinations thereof.

In some embodiments of any one of the aspects described herein, the redox mediator is ferrocene or a derivative thereof. As used herein, the term “ferrocene derivative” refers to a molecule containing an optionally substituted ferrocene group (e.g., optionally substituted ferrocenyl radical or ferrocene nucleus). Some exemplary ferrocene derivatives include, but are not limited to, aminoferrocene, ferrocene monocarboxylic acid, ferrocyanide, 1,1′-ferrocene dicarboxylic acid, 1,1′-dimethylferrocene (DMF), polyvinylferrocene, [N-ferrocenoyl]-4-aminophenyl phosphate, ferrocenylmethyl methacrylate, β-ferrocenyl-propenoic acid, and ferrocene monocarboxylic acid (FMCA). In some embodiments of any one of the aspects described herein, the redox mediator is aminoferrocene

In some embodiments of any one of the aspects described herein, the redox mediator is covalently linked with other components present in the composition or the antifouling coating layer. For example, the redox mediator can be covalently linked with the conductive element or the proteinaceous material in the in the composition or the antifouling coating layer. For example, the redox mediator is covalently linked to the proteinaceous material in the composition or the antifouling coating layer. In another non-limiting example, the redox mediator is covalently linked to the conductive element in the in the composition or the antifouling coating layer.

In some embodiments of any one of the aspects described herein, the redox mediator in the antifouling coating layer is covalently linked to the substrate surface, e.g., conductive substrate surface coated with the antifouling coating layer. It is noted the redox mediator can be linked directly to the surface without being linked to the conductive element or the proteinaceous material in the in the composition or the antifouling coating layer. In some embodiments, the redox mediator is not linked directly to the surface. For example, the redox mediator is linked to the surface by forming a covalent link to the conductive element or the proteinaceous material.

It is noted that the redox mediator can be covalently linked with the conductive element, the proteinaceous material or the substrate surface directly (e.g., a bond) or by a cross-linking agent. Exemplary cross-linking agents include, but are not limited to, glutaraldehyde, genipin, polyethylene glycol, carbodiimide based cross-linkers. Accordingly, in some embodiments, the redox mediator is covalently linked with the conductive element by a cross-linking agent. For example, the redox mediator covalently linked with the conductive element by a cross-linking agent selected from glutaraldehyde, genipin, polyethylene glycol, and carbodiimide cross-linker. In some embodiments, the redox mediator is covalently linked to the conductive element by formation of a bond between a functional group in the redox mediator and a complementary functional group in the conductive element. In some other embodiments, the redox mediator is covalently linked to the proteinaceous material by formation of a bond between a functional group in the redox mediator and a complementary functional group in the proteinaceous material.

The ratio of the redox mediator to the cross-linking agent can be from about 100:1 to about 1:1 (w/w). In some embodiments of any one of the aspects described herein, the ratio of the redox mediator to the cross-linker is from about 100:1 to about 10:1 (w/w). For example, the ratio of the redox mediator to the cross-linker can be from about 90:1 to about 20:1, about 80:1 to about 30:1, about 70:1 to about 40:1, or about 60:1 to about 50:1 (w/w). In some embodiments, the w/w ratio of the redox mediator to cross-linker is about 100:1, or about 95:1, or about 90:1, or about 85:1, or about 80:1, about 75:1, or about 70:1, or about 65:1, or about 60:1, about 55:1, or about 50:1, or about 45:1, or about 40:1, about 35:1, or about 30:1, or about 25:1, or about 20:1, or about 15:1, or about 10:1, or about 5:1 or about 1:1.

The redox mediator can be present in the composition or the antifouling coating layer in an amount from about 0.001 to about 10 (w/w).

The ratio of the redox mediator to the oxidoreductase in the composition or the antifouling coating layer can be from about 1:100 to about 1:1 (w/w). In some embodiments of any one of the aspects described herein, the ratio of the redox mediator to the oxidoreductase in the composition or the antifouling coating layer is from about 1:100 to about 1:10 (w/w). For example, the ratio of the redox mediator to the oxidoreductase in the composition or the antifouling coating layer can be from about 1:90 to about 1:20, about 1:80 to about 1:30, about 1:70 to about 1:40, or about 1:60 to about 1:50 (w/w). In some embodiments, the w/w ratio of the redox mediator to the oxidoreductase in the composition or the antifouling coating layer is about 1:100, or about 1:95, or about 1:90, or about 1:85, or about 1:80, about 1:75, or about 1:70, or about 1:65, or about 1:60, about 1:55, or about 1:50, or about 1:45, or about 1:40, about 1:35, or about 1:30, or about 1:25, or about 1:20, or about 1:15, or about 1:10, or about 1:5, or about 1:1.

The ratio of the redox mediator to the proteinaceous material in the composition or the antifouling coating layer can be from about 1:10 to about 1:1000 (w/w). In some embodiments of any one of the aspects described herein, the ratio of the redox mediator to the proteinaceous material in the composition or the antifouling coating layer is from about 1:10 to about 1:900 (w/w). For example, the ratio of the redox mediator to the proteinaceous material in the composition or the antifouling coating layer can be from about 1:100 to about 1:800, about 1:200 to about 1:700, about 1:300 to about 1:600, or about 1:400 to about 1:500 (w/w). In some embodiments, the w/w ratio of the redox mediator to the proteinaceous material in the composition or the antifouling coating is about 1:10, or about 1:50, or about 1:100, or about 1:150, or about 1:200, about 1:250, or about 1:300, or about 1:350, or about 1:400, about 1:450, or about 1:500, or about 1:550, or about 1:600, about 1:650, or about 1:700, or about 1:750, or about 1:800, or about 1:850, or about 1:900, or about 1:950, or about 1:1000.

The ratio of the redox mediator to the conductive element in the composition or the antifouling coating layer can be from about 1:10 to about 1:1000 (w/w). In some embodiments of any one of the aspects described herein, the ratio of the redox mediator to the conductive element in the composition or the antifouling coating layer is from about 1:10 to about 1:900 (w/w). For example, the ratio of the redox mediator to the conductive element in the composition or the antifouling coating layer can be from about 1:100 to about 1:800, about 1:200 to about 1:700, about 1:300 to about 1:600, or about 1:400 to about 1:500 (w/w). In some embodiments, the w/w ratio of the redox mediator to the conductive element in the composition or the antifouling coating is about 1:10, or about 1:50, or about 1:100, or about 1:150, or about 1:200, about 1:250, or about 1:300, or about 1:350, or about 1:400, about 1:450, or about 1:500, or about 1:550, or about 1:600, about 1:650, or about 1:700, or about 1:750, or about 1:800, or about 1:850, or about 1:900, or about 1:950, or about 1:1000.

### Oxidoreductases

Embodiments of the various aspects described herein include an oxidoreductase. As used herein, term “oxidoreductase” refers to any molecule, e.g., enzyme able to catalyze the transfer of electrons from one molecule (reductant, also called hydrogen acceptor or electron donor) to another (oxidant, also called hydrogen donor or electron acceptor). Exemplary oxidoreductases include, but are not limited to, (EC 1.1) oxidoreductases acting on the CH—OH group of donors, and an acceptor; (EC 1.2) oxidoreductases acting on the aldehyde or oxo group of donors, and an acceptor; (EC 1.3) oxidoreductases acting on the CH—CH group of donors, and an acceptor; (EC 1.4) oxidoreductases acting on the CH—NH₂ group of donors, and an acceptor; (EC 1.5) oxidoreductases acting on the CH—NH group of donors, and an acceptor; (EC 1.6) oxidoreductases acting on NADH or NADPH, and an acceptor; (EC 1.7) oxidoreductases acting on other nitrogenous compounds as donors, and an acceptor; (EC 1.8) oxidoreductases acting on a sulfur group of donors, and an acceptor; (EC 1.9) oxidoreductases acting on a heme group of donors, and an acceptor; (EC 1.1) oxidoreductases acting on diphenols and related substances as donors, and an acceptor; (EC 1.11) oxidoreductases acting on a peroxide as acceptor; (EC 1.12) oxidoreductases acting on hydrogen as donor, and an acceptor; (EC 1.13) oxidoreductases acting on single donors with incorporation of molecular oxygen, incorporating one or two oxygen atoms; (EC 1.14) oxidoreductases acting on paired donors, with incorporation or reduction of molecular oxygen, with the donor being 2-oxoglutarate, NADH, NADPH, reduced flavin, flavoprotein, pteridine, iron-sulfur protein, ascorbate; (EC 1.15) oxidoreductases acting on superoxide radicals as acceptor; (EC 1.16) oxidoreductases oxidizing metal ions, and an acceptor; (EC 1.17) oxidoreductases acting on CH or CH2 groups, and an acceptor; (EC 1.18) oxidoreductases acting on iron-sulfur proteins as donors, and an acceptor; (EC 1.19) oxidoreductases acting on reduced flavodoxin as donor, and an acceptor; (EC 1.2) oxidoreductases acting on phosphorus or arsenic in donors, and an acceptor; and (EC 1.21) oxidoreductases acting on X-H and Y-H to form an X-Y bond, and an acceptor; where acceptors for each donor category can include, without limitation: NAD, NADP, heme protein, oxygen, disulfide, quinone, an iron-sulfur protein, a flavin, a nitrogenous group, a cytochrome, dinitrogen, and H+.

In some embodiments of any one of the aspects described herein, the oxidoreductase can be selected from the group consisting of oxidases, reductases, dehydrogenases, peroxidases, oxygenases, monooxygenases, dioxygenases, lipoxygenases, hydrogenases, transhydrogenases, peroxidases, catalases, epoxidases, hydroxylases, demethylases, desaturases, dismutases, hydroxyltransferases, synthases, dehalogenases, and deiodinases. For example, the oxidoreductase is selected from the group consisting of glucose oxidase, malate oxidase, hexose oxidase, aryl-alcohol oxidase, alcohol oxidase, long-chain alcohol oxidase, glycerol-3-phosphate oxidase, poly vinyl-alcohol oxidase, D-arabinono-1,4-lactone oxidase, D-mannitol oxidase, xylitol oxidase, oxalate oxidase, carbon-monoxide oxidase, 4-hydroxyphenylpyruvate oxidase, dihydrouracil oxidase, ethanolamine oxidase, lactate oxidase, L-aspartate oxidase, sarcosine oxidase, urate oxidase, methanethiol oxidase, 3-hydroxyanthranilate oxidase, cholesterol oxidase, xanthine oxidase, amino-acid oxidase, laccase, catalase, fatty-acid peroxidase, peroxidase, diarylpropane peroxidase, ferroxidase, pteridine oxidase, columbamine oxidase, catechol 1,2-dioxygenase, gentisate 1,2-dioxygenase, homogentisate 1,2-dioxygenase, lipoxygenase, ascorbate 2,3-dioxygenase, 3-carboxyethylcatechol 2,3-dioxygenase, indole 2,3-dioxygenase, caffeate 3,4-dioxygenase, arachidonate 5-lipoxygenase, biphenyl-2,3-diol 1,2-dioxygenase, linoleate 11-lipoxygenase, acetylacetone-cleaving enzyme, lactate 2-monooxygenase, phenylalanine 2-monooxygenase, inositol oxygenase, fructose dehydrogenase, alcohol dehydrogenase, glycerol dehydrogenase, propanediol-phosphate dehydrogenase, L-lactate dehydrogenase, D-lactate dehydrogenase, glycerate dehydrogenase, glucose 1-dehydrogenase, galactose 1-dehydrogenase, allyl-alcohol dehydrogenase, 4-hydroxybutyrate dehydrogenase, octanol dehydrogenase, aryl-alcohol dehydrogenase, cyclopentanol dehydrogenase, long-chain-3-hydroxyacyl-CoA dehydrogenase, butanal dehydrogenase, terephthalate 1,2-cis-dihydrodiol dehydrogenase, succinate dehydrogenase, glutamate dehydrogenase, glycine dehydrogenase, hydrogen dehydrogenase, 4-cresol dehydrogenase, phosphonate dehydrogenase, diethyl 2-methyl-3-oxosuccinate reductase, tropinone reductase, long-chain-fatty-acyl-CoA reductase, carboxylate reductase, D-proline reductase, glycine reductlactase, Heme-proteins such as cytochromes, carbon-carbon lyases, carbon-oxygen lyases, carbon-nitrogen lyases, carbon-sulfur lyases, carbon-halide lyases, and phosphorus-oxygen lyases, alkaline phosphatase, malate dehydrogenase, staphylococcal nuclease, delta-V-steroid isomerase, yeast alcohol dehydrogenase, alpha-glycerophosphate dehydrogenase, triose phosphate isomerase, horseradish peroxidase (HRP), asparaginase, beta-galactosidase, ribonuclease, urease, catalase, glucose-VI-phosphate dehydrogenase, glucoamylase, tyrosinase, pyruvate oxidase, aldehyde oxidase, carbon-monoxide oxidase, retinal oxidase, abscisic-aldehyde oxidase, (methyl)glyoxal oxidase, xanthine oxidase, oxalate oxidase, and acetylcholinesterase.

In some embodiments of any one of the aspects described herein, the oxidoreductase is glucose oxidase.

In some embodiments of any one of the aspects described herein, the oxidoreductase is covalently linked with other components present in the composition or the antifouling coating layer. For example, the oxidoreductase can be covalently linked with the conductive element or the proteinaceous material in the composition or the antifouling coating layer. For example, the oxidoreductase is covalently linked to the proteinaceous material in the in the composition or the antifouling coating layer. In another non-limiting example, the oxidoreductase is covalently linked to the conductive element in the composition or the antifouling coating layer.

In some embodiments of any one of the aspects described herein, the oxidoreductase in the antifouling coating layer is covalently linked to the substrate surface, e.g., conductive substrate surface coated with the antifouling coating layer. It is noted the oxidoreductase can be linked directly to the surface without being linked to the conductive element or the proteinaceous material in the composition or the antifouling coating layer. In some embodiments, the oxidoreductase is not linked directly to the surface. For example, the oxidoreductase is linked to the surface by forming a covalent link to the conductive element or the proteinaceous material.

It is noted that the oxidoreductase can be covalently linked with the conductive element, the proteinaceous material or the substrate surface directly (e.g., a bond) or by a cross-linking agent. Exemplary cross-linking agents include, but are not limited to, glutaraldehyde, genipin, polyethylene glycol, and carbodiimide cross-linkers. Accordingly, in some embodiments, the oxidoreductase is covalently linked with the conductive element by a cross-linking agent. For example, the oxidoreductase covalently linked with the conductive element by a cross-linking agent selected from glutaraldehyde, genipin, polyethylene glycol, and carbodiimide cross-linkers. In some embodiments, the oxidoreductase is covalently linked to the conductive element by formation of a bond between a functional group in the oxidoreductase and a complementary functional group in the conductive element.

In some other embodiments, the oxidoreductase is covalently linked to the proteinaceous material by formation of a bond between a functional group in the oxidoreductase and a complementary functional group in the proteinaceous material. For example, the oxidoreductase is covalently linked to the proteinaceous material by formation of a bond between an amino group in the oxidoreductase and carboxylic acid in the proteinaceous material.

The ratio of the oxidoreductase to the cross-linking agent can be from about 100:1 to about 1:1 (w/w). In some embodiments of any one of the aspects described herein, the ratio of the oxidoreductase to the cross-linker is from about 100:1 to about 10:1 (w/w). For example, the ratio of the oxidoreductase to the cross-linker can be from about 90:1 to about 20:1, about 80:1 to about 30:1, about 70:1 to about 40:1, or about 60:1 to about 50:1 (w/w). In some embodiments, the w/w ratio of the oxidoreductase to cross-linker is about 100:1, or about 95:1, or about 90:1, or about 85:1, or about 80:1, about 75:1, or about 70:1, or about 65:1, or about 60:1, about 55:1, or about 50:1, or about 45:1, or about 40:1, about 35:1, or about 30:1, or about 25:1, or about 20:1, or about 15:1, or about 10:1, or about 5:1 or about 1:1.

The oxidoreductase can be present in the composition or the antifouling coating layer in an amount from about 20% to about 80% (w/v).

The ratio of the oxidoreductase to the proteinaceous material in the composition or the antifouling coating layer can be from about 10:1 to about 5:1 (w/w). In some embodiments of any one of the aspects described herein, the ratio of the oxidoreductase to the proteinaceous material in the composition or the antifouling coating layer is from about 10:1 to about 5:1 (w/w). For example, the ratio of the oxidoreductase to the proteinaceous material in the composition or the antifouling coating layer can be from about 9:1 to about 6:1, or about 8:1 to about 7:1 (w/w). In some embodiments, the w/w ratio of the oxidoreductase to the proteinaceous material in the composition or the antifouling coating is about 10:1, or about 9.5:1, or about 9:1, or about 8.5:1, or about 8:1, or about 7.5:1, or about 7:1, or about 6.5:1, or about 6:1, or about 5.5:1, or about 5:1.

The ratio of the oxidoreductase to the conductive element in the composition or the antifouling coating layer can be from about 10:1 to about 5:1 (w/w). In some embodiments of any one of the aspects described herein, the ratio of the oxidoreductase to the conductive element in the composition or the antifouling coating layer is from about 10:1 to about 5:1 (w/w). For example, the ratio of the oxidoreductase to the conductive element in the composition or the antifouling coating layer can be from 9:1 to about 6:1, or about 8:1 to about 7:1 (w/w). In some embodiments, the w/w ratio of the oxidoreductase to the conductive element in the composition or the antifouling coating is about 10:1, or about 9.5:1, or about 9:1, or about 8.5:1, or about 8:1, or about 7.5:1, or about 7:1, or about 6.5:1, or about 6:1, or about 5.5:1, or about 5:1.

### Proteinaceous Material

Embodiments of the various aspects described herein include a proteinaceous material. As used herein, the term “proteinaceous” refers to proteins, peptides and the like. Generally, proteinaceous material includes proteins and peptides, functionalized proteins, copolymers including proteins, natural and synthetic variants of these, and mixtures of these.

In some embodiments, the proteinaceous material is a globular protein. Exemplary globular proteins include, but are not limited to, albumin, Immunoglobulin G (IgG), Immunoglobulin E (IgE), Protein A, avidins, and carbonic anhydrase.

It is noted that the proteinaceous material can be a glycosylated protein or a non-glycosylated protein.

In some embodiments of any one of the aspects described herein, the proteinaceous material is a serum albumin. For example, the proteinaceous material can be Bovine Serum Albumin (BSA) or human serum albumin (HSA). In some embodiments of any one of the aspects described herein, the proteinaceous material is BSA.

In some embodiments, the proteinaceous material is denatured. As used herein, “denaturing” is the process of modifying the quaternary, tertiary and secondary molecular structure of a protein from its natural, original or native state. For example, such as by breaking weak bonds (e.g., hydrogen bonds), which are responsible for the highly ordered structure of the protein in its natural state. The process can be accomplished by, for example: physical means, such as by heating, sonication or shearing; by chemical means such as acid, alkali, inorganic salts and organic solvents (e.g., alcohols, acetone or chloroform); and by radiation. A denatured protein, such as an enzyme, losses its original biological activity. In some instances, the denaturing process is reversible, such that the protein molecular structure is regained by the re-forming of the original bonding interactions at least to the degree that the original biological function of the protein is restored. In other instances, the denaturing process is irreversible or non-reversible, such that the original and biological function of the protein is not restored. Cross-linking, for example after denaturing, can reduce or eliminate the reversibility of the denaturing process.

The degree of denaturing can be expressed as a percent of protein molecules that have been denatured, such as a mole percent. Some methods of denaturing can be more efficient than others. For example, under some conditions, sonication applied to a protein, e.g., serum albumin such as BSA or HSA can denature about 30-40% of the protein and the denaturing is reversible. When BSA or HAS is denatured it undergoes two structural stages. The first stage is reversible whilst the second stage is irreversible (e.g., non-reversible) but does not necessarily result in a complete destruction of the ordered structure. For example, heating up to 65° C. can be regarded as the first stage, with subsequent heating above that as the second stage. At higher temperatures, further transformations are seen. In some embodiments, the proteinaceous material e.g., serum album such as BSA or HAS is denatured by heating above about 65° C. (e.g., above about 70° C., above about 80° C., above about 90° C., above about 100° C., above about 110° C., above about 120° C.), below about 200° C. (below about 190° C., 180° C., 170° C., 160° C., 150° C.), and for at least about 1 minute (e.g., at least about 2, 3, 4, 5, 10 or 20 minutes) but less than about 24 hours (e.g., less than about 12, 10, 8, 6, 4, 2 1 hour). According to some implementations, any ranges herein described, for example heating at about 90° C. but below about 150° C. and for at least about 1 minute but less than one hour. As previously noted, the heating can include be included as a separate step to heating of the substrate and include different temperature ranges and heating times.

In some embodiments of any one of the aspects, denaturing of the proteinaceous material can occur before deposition of the mixture on a substrate surface. In some embodiments of any one of the aspects, denaturing can occur only upon deposition on the substrate surface, for example when only a heating step to heat the substrate is included. In some implementations, denaturing occurs before and after deposition, for example, where heating occurs before and after deposition of the mixture on the substrate surface.

In some embodiments the proteinaceous material used in the compositions and coating layers described herein are at least about 20% to about 100% (e.g., 20%, 30%, 40%, 50%, 60%, 70%, 80%, 90%, or 100%) denatured. In some embodiments, less than 50% of the denatured protein reverts back to its natural state (e.g., less than 40%, less than 30%, less than 20%, less than 10%, less than 1%). Therefore, the reversibility of the denaturing can be described as being 50% reversible, 40% reversible (60% irreversible), 30% reversible (70% irreversible), 20% reversible (80% irreversible), 10% reversible (90% irreversible) or even 0% reversible (100% irreversible).

In some embodiments, the proteinaceous material is irreversibly or non-reversibly denatured.

The proteinaceous material can be cross-linked with other components present in the composition or the antifouling coating layer. For example, the proteinaceous material can be cross-linked with the redox mediator, the oxidoreductase, the conductive element, or itself. Accordingly, in some embodiments, the proteinaceous material is cross-linked with the conductive element. For example, the proteinaceous material is cross-linked to the conductive element by a cross-linking agent. In some embodiments of any one of the aspects described herein, the proteinaceous material is cross-linked to the conductive element by a cross-linking agent selected from genipin, polyethylene glycol, glutaraldehyde, and carbodiimide cross-linkers. For example, the proteinaceous material is cross-linked to the conductive element by genipin.

In some embodiments, the proteinaceous material is cross-linked to itself. For example, the proteinaceous material is cross-linked to the conductive element by a cross-linking agent. In some embodiments of any one of the aspects described herein, the proteinaceous material is cross-linked to itself via a cross-linking agent selected from genipin, polyethylene glycol, glutaraldehyde, and carbodiimide cross-linkers. For example, the proteinaceous material is cross-linked to itself via genipin.

In some embodiments, the proteinaceous material in the antifouling coating layer is covalently linked to the surface that is coated by the antifouling coating layer.

The ratio of the proteinaceous material to the cross-linking agent can be from about 100:1 to about 1:1 (w/w). In some embodiments of any one of the aspects described herein, the ratio of the proteinaceous material to the cross-linker is from about 100:1 to about 10:1 (w/w). For example, the ratio of the proteinaceous material to the cross-linker can be from about 90:1 to about 20:1, about 80:1 to about 30:1, about 70:1 to about 40:1, or about 60:1 to about 50:1 (w/w). In some embodiments, the w/w ratio of proteinaceous material to cross-linker is about 100:1, or about 95:1, or about 90:1, or about 85:1, or about 80:1, about 75:1, or about 70:1, or about 65:1, or about 60:1, about 55:1, or about 50:1, or about 45:1, or about 40:1, about 35:1, or about 30:1, or about 25:1, or about 20:1, or about 15:1, or about 10:1, or about 5:1 or about 1:1.

The ratio of proteinaceous material to the conductive element in the composition or the antifouling coating layer can be from about 10:1 to 1:1 (w/w). For example, the w/w ratio of ratio of the proteinaceous material to the conductive element in the composition or the antifouling coating layer is about 10:1, or about 9.5:1, or about 9:1, or about 8.5:1, or about 8:1, or about 7.5:1, or about 8:1, or about 6.5:1, or about 6:1 or about 5.5:1, or about 5:1, or about 4.5:1, or about 4:1, or about 3.5:1 or about 3:1, or about 2.5:1, or about 2:1, or about 1.5:1 or about 1:1 (w/w).

The amount of the proteinaceous material in the composition or the antifouling coating layer can range from about 1 mg/ml to about 20 mg/ml. For example, the amount of the proteinaceous material in the composition or the antifouling coating layer can be about 1 mg/ml, about 1.5 mg/ml, about 2 mg/ml, about 2.5 mg/ml, about 3 mg/ml, about 3.5 mg/ml, about 4 mg/ml, about 4.5 mg/ml, about 5 mg/ml, about 5.5 mg/ml, about 6 mg/ml, about 6.5 mg/ml, about 7 mg/ml, about 7.5 mg/ml, about 8 mg/ml, about 8.5 mg/ml, about 9 mg/ml, about 9.5 mg/ml, about 10 mg/ml, about 10.5 mg/ml, about 11 mg/ml, about 11.5 mg/ml, about 12 mg/ml, about 12.5 mg/ml, about 13 mg/ml, about 13.5 mg/ml, about 14 mg/ml, about 14.5 mg/ml, about 15 mg/ml, about 15.5 mg/ml, about 16 mg/ml, about 16.5 mg/ml, about 17 mg/ml, about 17.5 mg/ml, about 18 mg/ml, about 18.5 mg/ml, about 19 mg/ml, about 19.5 mg/ml, or about 20 mg/ml. In some embodiments of any one of the aspects described herein, the amount of the proteinaceous material in the composition or the antifouling coating layer is from about 2 mg/ml to about 18 mg/ml, about 3 mg/ml to about 17 mg/ml, about 4 mg/ml to about 16 mg/ml. For example, the amount of the proteinaceous material in the composition or the antifouling coating layer is from about 5 mg/ml to about 15 mg/ml. In some embodiments, the amount of the proteinaceous material in the composition or the antifouling coating layer is from about 5 mg/ml to about 10 mg/ml.

In some embodiments, the amount of the proteinaceous material in the composition or the antifouling coating layer can range from about 0.1% to about 20% (w/v, w/w or v/v), e.g., from about 0.1% to about 10% (w/v, w/w, or v/v). For example, the amount of the proteinaceous material in the composition or the antifouling coating layer can be about 0.1%, about 0.125%, about 0.15%, about 0.175%, about 0.2%, about 0.225%, about 0.125%, about 0.275%, about 0.3%, about 0.325%, about 0.35%, about 0.375%, about 0.4%, about 0.425%, about 0.45%, about 0.475%, about 0.5%, about 0.525%, about 0.55%, about 0.575%, about 0.6%, about 0.625%, about 0.65%, about 0.675%, about 0.7%, about 0.725%, about 0.75%, about 0.775%, about 0.8%, about 0.825%, about 0.85%, about 0.875%, about 0.9%, about 0.925%, about 0.95%, about 0.975%, about 1%, about 1.125%, about 1.15%, about 1.175%, about 1.2%, about 1.225%, about 1.125%, about 1.275%, about 1.3%, about 1.325%, about 1.35%, about 1.375%, about 1.4%, about 1.425%, about 1.45%, about 1.475%, about 1.5%, about 1.525%, about 1.55%, about 1.575%, about 1.6%, about 1.625%, about 1.65%, about 1.675%, about 1.7%, about 1.725%, about 1.75%, about 1.775%, about 1.8%, about 1.825%, about 1.85%, about 1.875%, about 1.9%, about 1.925%, about 1.95%, about 1.975%, or about 2% (w/v, w/w or v/v). In some embodiments of any one of the aspects described herein, the amount of the proteinaceous material in the composition or the antifouling coating layer is from about 0.25% to about 1.75%, about 0.5% to about 1.5%, or about 0.75% to about 1.25% (w/v, w/w or v/v). For example, the amount of the proteinaceous material in the composition or the antifouling coating layer is about 1% (w/v, w/w or v/v).

### Conductive Element

Embodiments of the various aspects described herein include a conductive element. As used herein a conductive element is a substance or substrate that has the capability to conduct electricity. The conductive element can comprise conducting and/or semi-conducting materials. Further, the conductive element can be in any desired shape or form. For example, the conductive element can be in form of particles (e.g., nanoparticles), rods, flakes (e.g., nanoflakes), tubes (e.g., nanotubes), fibers, sheets, films, and the like. For example, the conductive element can be included in the form of a particle, a nano-particle, a micro-particle, a fiber, a nano-fiber, a micro-fiber, a flake, a nanoflake, a microflake, a tube, a nanotube, a microtube, a crystal, a nanocrystal, a microcrystal, a wire, a nano-wire, a micro-wire, a rod, a nano-rod, a micro-rod, a foil, a sheet, a web, or any combinations of these forms.

The conductive element can be formed from one or more metals, e.g., copper, gold, silver, platinum, palladium, indium, iridium, rhodium, ruthenium, osmium, nickel, tin, titanium, tantalum, tungsten, chromium, iron, aluminum, zinc, combinations thereof, or alloys of any of the foregoing. In addition, or in the alternative, a nonmetallic conductive material can be used. Exemplary nonmetallic conductive materials include, but are not limited to, graphite or acetylene black, graphene, conductive ceramics such as indium tin oxide (ITO), titanium nitride, tungsten nitride, tantalum nitride, and conductive polymers such as polythiophenes, polyanilines, polypyrroles, and polyethylenes and their mixtures and derivatives.

In some embodiments of any one of the aspects, the conductive element comprises a metal or a metalloid. For example, the conductive element comprises gold. In some embodiments of any one of the aspects described herein, the conductive element comprises gold particles (e.g., gold nano-particles), gold wires (e.g., gold nanowires), gold rods (e.g., gold nano-rods), or any combinations thereof.

In some embodiments of any one of the aspects described herein, the conductive element comprises a conducting carbon-based material. For example, the conductive element comprises an allotrope of carbon atoms arranged in a hexagonal lattice. The allotropes of carbon can include some functionalization, such as oxygen, carboxylates, epoxides, amines, amides and combinations of these, as described below. In some implementations, the functionalization includes poly amine functionalization such as pentaamine functionalization. In some embodiments, the conductive element comprises graphite, graphene, graphene oxide, functionalized graphene oxide, reduced graphene oxide (rGO), functionalized reduced graphene oxide, or carbon nano-tubes (CNTs).

As used herein “carbon nanotubes” and “graphene” are allotropes of carbon with sp² carbon atoms arranged in a hexagonal, honeycomb lattice. Single layer graphene is a two-dimensional material, and is a single layer of graphite. As used herein, more than one layer of graphene can be referred to as graphene, for example between 1 and 200 layers (e.g., about 1 to 100 layers, about 1 to 50 layers, about 1 to 10 layers). Carbon nanotubes are hollow, cylindrical structures, formed as a sheet of graphene rolled into a cylinder.

As used herein “graphene oxide” is a material that can be formed from the oxidation of graphene or exfoliation of graphite oxide. In a first step for producing graphene oxide, graphite is oxidized. Several methods for oxidation are known, one common method known as the Hummers and Offeman method, in which graphite is treated with a mixture of sulphuric acid, sodium nitrate and potassium permanganate (a very strong oxidizer). Other methods are known to be more efficient, reaching levels of 70% oxidisation, by using increased quantities of potassium permanganate, and adding phosphoric acid combined with the sulphuric acid, instead of adding sodium nitrate. Exfoliation of graphene oxide provides graphite oxide and can be done by several methods. Sonication can be a very time-efficient way of exfoliating graphite oxide, and it is extremely successful at exfoliating graphene (almost to levels of full exfoliation), but it can also heavily damage the graphene flakes, reducing them in surface size from microns to nanometres, and also produces a wide variety of graphene platelet sizes. Mechanically stirring is a much less destructive approach, but can take much longer to accomplish.

Graphite oxide and graphene oxide are very similar, chemically, but structurally, they are very different. Both are compounds having carbon, oxygen and hydrogen in variable ratios. In the most oxidized state the oxygen amount can be as high as about 60 wt %. the amount of hydrogen varies depending on the functionalization, for example, the number of epoxy bridges, hydroxyl groups and carboxyl groups. The main difference between graphite oxide and graphene oxide is the interplanar spacing between the individual atomic layers of the compounds, caused by water intercalation. This increased spacing, caused by the oxidization process, also disrupts the sp² bonding network, meaning that both graphite oxide and graphene oxide are often described as electrical insulators.

Reduced graphene oxide (rGO) is prepared from reduction of graphene oxide by thermal, chemical or electrical treatments. For example, treating the graphene oxide with hydrazine, hydrogen plasma, heating in water, high temperature heating (e.g., under nitrogen/argon) and electrochemical reduction. Whereas graphene can be a single carbon layer ideally comprising only carbon, reduced graphene oxide is similar but contains some degree of oxygen functionalization. The amount of oxygen depends on the degree of reduction and in some materials can vary between about 50 wt % and about 1 wt. % (e.g., between about 30 wt. % and about 5 wt. %).

Reduced graphene oxide can be functionalized or include functional groups. For example, reduced graphene oxide often includes oxygen in the form of carboxyl groups and hydroxyl groups. In some forms, the carboxyl and hydroxyl groups populate the edges of the rGO sheets, which can be functionalized. Accordingly, in some embodiments, the reduced graphene oxide (rGO) is carboxylated reduced graphene oxide or aminated reduced graphene oxide. As used herein, carbonylated reduced graphene oxide can refer to reduced graphene oxide having carboxyl groups. In some embodiments the amount of oxygen attributable to the carboxyl groups is between about 30 wt. % and about 0.1 wt. % (e.g., between about 10 wt. % and about 1 wt. %). Other forms of functionalization are possible. For example, amine functionalized rGO can be formed by a modified Buchere reaction, wherein ammonia an graphene oxide are reacted using a catalyst such as sodium bisulfite, or epoxide groups on graphene oxide can be opened with p-phenylenediamine. In some embodiments, the amount of nitrogen is between about 30 wt. % and 0.1 wt. % (e.g., between about 10 wt. % and 1 wt. %). In some implementations, a polyamine is used to functionalize rGO. For example, pentaamine functionalized graphene is used in some implementations.

The tube-shaped carbon nanotubes have diameters in the nanometer scale, such as, for example, between about 0.2 and about 20 nm, preferably between about 0.5 and about 10 nm, and more preferably still between about 1 and about 5 nm. These can be single walled carbon nanotubes (SWCNT), multi walled carbon nanotubes (MWCNT) (e.g., a collection of 2 or more nested tubes of continuously increasing diameters, or mixtures of these). The diameters of MWCNT can be larger than the SWCNT, such as between about 1 and about 100 nm (e.g., between about 1 and about 50 nm, between about 10 and 20 nm, between 5 and 15 nm, between about 30 and 50 nm). Depending on how the precursor graphene sheet is rolled up to make a seamless cylinder that is the carbon nanotube, different isomers of carbon nanotube can be made, for example designated as armchair configuration, chiral configuration, and zigzag configuration. In some embodiments, the carbon nanotubes are carboxylated carbon nanotubes or aminated carbon nanotubes.

The carbon nanotubes and reduced graphene oxide can include intercalated materials, such as ions and molecules. In some embodiments the carbon nanotubes can be functionalized for example by oxidation to form carboxylic acid groups on the surface, providing CNTs. In addition, in some embodiments, the carbon nanotubes and rGO can be further modified through condensation reactions with the carboxylic acid groups present on the CNTs or rGO (e.g., with alcohols and amines), electrostatic interactions with the carboxylic acid groups (e.g., calcium mediated coupling, or quaternary amines, protonated amine-carboxylate interaction, through cationic polymers or surfactants) or hydrogen bonding through the carboxylic acid groups (e.g., with fatty acids, and other hydrogen bonding molecules). The functionalization can be partial (e.g., wherein less than 90%, less than 80%, less than 60%, less than 50%, less than 40%, less than 30%, less than 20%, less than 10%, more than 10%, more than 20%, more than 30%, more than 40%, more than 50%, more than 60%, more than 70%, more than 80%, of the available carboxylic acid groups are functionalized) or complete, such as functionalizing substantially all the carboxylic acids (e.g., more than 90%, more than 95%, more than 99% of available carboxylic acid groups).

In some embodiments of any one of the aspects, the conductive element comprises a conductive polymer. Exemplary conductive polymers include, but are not limited to, polyacrylonitrile (PAN), polyanilines, polypyrroles, polyacetylenes, polyphenylene sulfide, polythiophene, polyfluorene, polypyrene, polyazulene, polynaphthalene, polycarbazole, polyindole, polyazepine, poly(3, 4-ethylenedioxythiophene) (PEDOT), poly(p-phenylene sulfide) (PPS), poly(p-phenylene vinylene), poly(fluorenes)s, polyphenylenes, polypyrenes, polyazulenes, polynaphthalenes, polyanilines, polyazepines, polyindoles, polycarbazoles, poly(pyrrole)s, poly(thiophene)s, poly (p-phenylene vinylene) (PPV), and mixtures thereof.

In some embodiments of any one of the aspects described herein, the conductive element comprises one or more organic compounds having conducting and/or semiconducting properties. Exemplary organic compounds having conducting and/or semiconducting properties include, but are not limited to polyacetylenes, porphyrins, phthalocyanins, fullerenes, polyparaphenylenes, polyphenylenevinylenes, polyfluorenes, polythiophenes, polypyrroles, polypyridines, polycarbazoles, polypyridinevinylenes, polyarylvinylenes, poly(p-phenylmethylvinylenes), derivatives and co-polymers thereof, and any combination thereof. In some embodiments of any one of the aspects described herein, the organic compounds having conducting and/or semiconducting properties are selected from the group consisting of poly(3-hexylthiophene) (P3HT), perfluoro-3,6-dioxa-4-methyl-7-octenesulfonic acid copolymer, tetrafluoroethylene-perfluoro-3,6-dioxa-4-methyl-7-octenesulfonic acid copolymer (e.g. nafion), poly(9,9-dioctylfluorene-2,7-diyl-co-bis-N,N-(4-butylphenyl)-bis-N,N-phenyl-1,4-phenylenediamine), poly(9,9-dioctylfluorene-2,7-diyl-co-benzothiadiazole), (6,6)-phenyl-C61-butyric acid methyl ester, poly(2-methoxy-5-(2′-ethyl-hexyloxy)-1,4-phenylene vinylene), poly(4-vinylphenol) (PVP), and copolymers thereof, and any combination thereof.

The conductive element can be cross-linked with other components present in the in the composition or the antifouling coating layer. For example, the conductive element can be cross-linked with the antimicrobial agent, the proteinaceous material, or itself. In some embodiments, the conductive element is cross-linked with another component of the mixture or by a cross-linking agent. In some embodiments of any one of the aspects described herein, the conductive element is cross-linked with another component of the mixture or to itself by a cross-linking agent by a cross-linking agent selected from genipin, polyethylene glycol, glutaraldehyde, and carbodiimide cross-linkers. For example, the conductive element is cross-linked with another component of the mixture or to itself by genipin.

In some embodiments, the conductive element in the antifouling coating layer is covalently linked to the surface that is coated by the antifouling coating layer.

The ratio of the conductive element to the cross-linking agent can be from about 100:1 to about 1:1 (w/w). In some embodiments of any one of the aspects described herein, the ratio of the conductive element to the cross-linker is from about 100:1 to about 10:1 (w/w). For example, the ratio of the proteinaceous material to the cross-linker can be from about 90:1 to about 20:1, about 80:1 to about 30:1, about 70:1 to about 40:1, or about 60:1 to about 50:1 (w/w). In some embodiments, the w/w ratio of the conductive element to cross-linker is about 100:1, or about 95:1, or about 90:1, or about 85:1, or about 80:1, about 75:1, or about 70:1, or about 65:1, or about 60:1, about 55:1, or about 50:1, or about 45:1, or about 40:1, about 35:1, or about 30:1, or about 25:1, or about 20:1, or about 15:1, or about 10:1, or about 5:1 or about 1:1.

The amount of the conductive element in the composition or the antifouling coating layer can range from about 1 mg/ml to about 20 mg/ml. For example, the amount of the conductive element in the composition or the antifouling coating layer can be about 1 mg/ml, about 1.5 mg/ml, about 2 mg/ml, about 2.5 mg/ml, about 3 mg/ml, about 3.5 mg/ml, about 4 mg/ml, about 4.5 mg/ml, about 5 mg/ml, about 5.5 mg/ml, about 6 mg/ml, about 6.5 mg/ml, about 7 mg/ml, about 7.5 mg/ml, about 8 mg/ml, about 8.5 mg/ml, about 9 mg/ml, about 9.5 mg/ml, about 10 mg/ml, about 10.5 mg/ml, about 11 mg/ml, about 11.5 mg/ml, about 12 mg/ml, about 12.5 mg/ml, about 13 mg/ml, about 13.5 mg/ml, about 14 mg/ml, about 14.5 mg/ml, about 15 mg/ml, about 15.5 mg/ml, about 16 mg/ml, about 16.5 mg/ml, about 17 mg/ml, about 17.5 mg/ml, about 18 mg/ml, about 18.5 mg/ml, about 19 mg/ml, about 19.5 mg/ml, or about 20 mg/ml. In some embodiments of any one of the aspects described herein, the amount of the conductive element in the composition or the antifouling coating layer is from about 2 mg/ml to about 18 mg/ml, about 3 mg/ml to about 17 mg/ml, about 4 mg/ml to about 16 mg/ml. For example, the amount of the conductive element in the composition or the antifouling coating layer is from about 5 mg/ml to about 15 mg/ml. In some embodiments, amount of the conductive element in the composition or the antifouling coating layer is from about 5 mg/ml to about 10 mg/ml.

In some embodiments, the amount of the conductive element in the composition or the antifouling coating layer can range from about 0.01 to about 20% (w/v, w/w or v/v), e.g., 0.01% to about 10% (w/v, w/w or v/v). For example, the amount of the conductive element in the composition or the antifouling coating layer can be about 0.01%, about 0.0125%, about 0.015%, about 0.0175%, about 0.02%, about 0.0225%, about 0.0125%, about 0.0275%, about 0.03%, about 0.0325%, about 0.035%, about 0.0375%, about 0.04%, about 0.0425%, about 0.045%, about 0.0475%, about 0.05%, about 0.0525%, about 0.055%, about 0.0575%, about 0.06%, about 0.0625%, about 0.065%, about 0.0675%, about 0.07%, about 0.0725%, about 0.075%, about 0.0775%, about 0.08%, about 0.0825%, about 0.085%, about 0.0875%, about 0.09%, about 0.0925%, about 0.095%, about 0.0975%, about 0.1%, about 0.125%, about 0.15%, about 0.175%, about 0.2%, about 0.225%, about 0.125%, about 0.275%, about 0.3%, about 0.325%, about 0.35%, about 0.375%, about 0.4%, about 0.425%, about 0.45%, about 0.475%, about 0.5%, about 0.525%, about 0.55%, about 0.575%, about 0.6%, about 0.625%, about 0.65%, about 0.675%, about 0.7%, about 0.725%, about 0.75%, about 0.775%, about 0.8%, about 0.825%, about 0.85%, about 0.875%, about 0.9%, about 0.925%, about 0.95%, about 0.975%, about 1%, about 1.125%, about 1.15%, about 1.175%, about 1.2%, about 1.225%, about 1.125%, about 1.275%, about 1.3%, about 1.325%, about 1.35%, about 1.375%, about 1.4%, about 1.425%, about 1.45%, about 1.475%, about 1.5%, about 1.525%, about 1.55%, about 1.575%, about 1.6%, about 1.625%, about 1.65%, about 1.675%, about 1.7%, about 1.725%, about 1.75%, about 1.775%, about 1.8%, about 1.825%, about 1.85%, about 1.875%, about 1.9%, about 1.925%, about 1.95%, about 1.975%, or about 2% (w/v, w/w or v/v). In some embodiments of any one of the aspects described herein, the amount of the conductive element in the composition or the antifouling coating layer is from about 0.01% to about 2%, about 0.05% to about 1.75%, about 0.1% to about 1.5%, about 0.35% to about 1%, or about 0.25% to about 0.75% (w/v, w/w or v/v). For example, the amount of the conductive element in the composition or the antifouling coating layer is about 0.5% (w/v, w/w or v/v).

### Additional Components—Target Binding Molecule

The electrodes and sensors described herein can be combined with affinity-based sensors to create multiplexed electrochemical sensor-based diagnostics that detect multiple different types of analytes (e.g., protein levels as well as metabolite levels) simultaneously. Accordingly, in some embodiments of any one of the aspects described herein, the composition and/or the antifouling coating layer described herein further comprises a target binding molecule. The terms “target binding ligand” and “target binding molecule” are used interchangeably herein and refer to a molecule that binds to or interacts with a target molecule. In other words, a target binding ligand or molecule is a molecule that is capable of binding with a target molecule. The targeting binding ligand can be a natural or synthetic molecule (e.g., a molecular receptor) that binds to a target molecule. Exemplary target binding ligands include, but are not limited to, a receptor, a ligand for a receptor, an antibody, an antigen binding fragment of an antibody, an antigen, an enzyme, or a nucleic acid. The target binding ligand is also referred to as a “capture agent” or “capture molecule” herein.

In some embodiments of any one of the aspects described herein, the binding of the target binding ligand to the target molecule is a specific binding such that it is selective to that target above non-targets. For example the dissociation constant between the target binding ligand and target molecule is at least about 200 nM, alternatively at least about 150 nM, alternatively at least about 100 nM, alternatively at least about 60 nM, alternatively at least about 50 nM, alternatively at least about 40 nM, alternatively at least about 30 nM, alternatively at least about 20 nM, alternatively at least about 10 nM, alternatively at least about 8 nM, alternatively at least about 6 nM, alternatively at least about 4 nM, alternatively at least about 2 nM, alternatively at least about 1 nM, or greater. In certain embodiments, the specific binding refers to binding where the target binding ligand binds to its target molecule without substantially binding to any other species in the sample/test solution.

By way of non-limiting examples, a target binding ligand can be selected from antibodies, adnectins, ankyrins, other antibody mimetics and other protein scaffolds, aptamers, nucleic acid (e.g., an RNA or DNA aptamer), protein, peptide, binding partner, oligosaccharides, polysaccharides, lipopolysaccharides, cellular metabolites, cells, viruses, subcellular particles, haptens, pharmacologically active substances, alkaloids, steroids, vitamins, amino acids, avimers, peptidomimetics, hormone receptors, cytokine receptors, synthetic receptors, sugars or molecularly imprinted polymer. The target binding ligand can be selective to a specific target or class of targets such as toxins and biomolecules. For example, the target can be ions, molecules, oligomers, polymers, proteins, peptides, nucleic acids, toxins, biological threat agents such as spore, viral, cellular and protein toxins, carbohydrates (e.g., mono saccharides, disaccharides, oligosaccharides, polyols, and polysaccharides) and combinations of these (e.g., copolymers including these).

In some embodiments of any one of the aspects described herein, the target binding ligand is an antibody or antigen binding fragment thereof. As used herein, the terms “antibody” and “antibodies” include polyclonal antibodies, monoclonal antibodies, humanized or chimeric antibodies, single chain Fv antibody fragments, Fab fragments, and F(ab)₂ fragments. Antibodies having specific binding affinity for a target of interest (e.g., an antigen) can be produced through standard methods. As used herein, the terms “antibody” and “antibodies” refer to intact antibody, or a binding fragment thereof that competes with the intact antibody for specific binding and includes chimeric, humanized, fully human, and bispecific antibodies. In some embodiments, binding fragments are produced by recombinant DNA techniques. In additional embodiments, binding fragments are produced by enzymatic or chemical cleavage of intact antibodies. Binding fragments include, but are not limited to, Fab, Fab′, F(ab′)₂, Fv, and single-chain antibodies.

### Additional Components—Antimicrobial Agents

The compositions and antifouling coating layers described herein can comprises additional components. For example, the composition and/or the antifouling layer can further comprise an antimicrobial agent. The term “antimicrobial agent” as used herein refers to any entity with antimicrobial activity, i.e. the ability to inhibit or reduce the growth and/or kill a microbe, e.g., by at least about 30%, at least about 40%, at least about 50%, at least about 75%, at least about 90% or more, as compared to in the absence of an antimicrobial agent.

It is noted that the antimicrobial agent can be present in the antifouling coating layer or at a surface of the antifouling coating layer. Accordingly, in some embodiments of any one of the aspects described herein, the antimicrobial agent molecule is on a surface of the antifouling coating layer. In some embodiments of any one of the aspects described herein, the antimicrobial agent is embedded within the antifouling coating layer. In some embodiments of any one of the aspects described herein, the antimicrobial agent is imprinted on the antifouling coating layer. In some embodiments of any one of the aspects described herein, the antimicrobial agent is in pores of the antifouling coating layer.

In some embodiments, the anti-microbial agent is an anti-bacterial agent, antifungal agent, or an anti-viral agent. In some embodiments of any one of the aspects described herein, the anti-microbial agent is an antimicrobial peptide or polymer. In some embodiments of any one of the aspects described herein, the anti-microbial agent is a metal particle, e.g., oxide, copper, or silver nanoparticles.

In some embodiments, an antimicrobial agent included in the composition and/or the antifouling coating layer can be an antibiotic. As used herein, the term “antibiotic” is art recognized and includes antimicrobial agents naturally produced by microorganisms such as bacteria (including Bacillus species), actinomycetes (including Streptomyces) or fungi that inhibit growth of or destroy other microbes, or genetically-engineered thereof and isolated from such natural source. Substances of similar structure and mode of action can be synthesized chemically, or natural compounds can be modified to produce semi-synthetic antibiotics. Exemplary classes of antibiotics include, but are not limited to, (1) β-lactams, including the penicillins, cephalosporins monobactams, methicillin, and carbapenems; (2) aminoglycosides, e.g., gentamicin, kanamycin, neomycin, tobramycin, netilmycin, paromomycin, and amikacin; (3) tetracyclines, e.g., doxycycline, minocycline, oxytetracycline, tetracycline, and demeclocycline; (4) sulfonamides (e.g., mafenide, sulfacetamide, sulfadiazine and sulfasalazine) and trimethoprim; (5) quinolones, e.g., ciprofloxacin, norfloxacin, and ofloxacin; (6) glycopeptides (e.g., vancomycin, telavancin, teicoplanin); (7) macrolides, which include for example, erythromycin, azithromycin, and clarithromycin; (8) carbapenems (e.g., ertapenem, doripenem, meropenem, and imipenem); (9) cephalosporins (e.g., cefadroxil, ceftriaxone, cefepime, and ceftobiprole); (10) lincosamides (e.g., clindamycin, and lincomycin); (11) monobactams (e.g., aztreonam); (12) nitrofurans (e.g., furazolidone, and nitrofurantoin); (13) Penicillins (e.g., amoxicillin, and Penicillin G); (14) polypeptides (e.g., bacitracin, colistin, and polymyxin B); and (15) other antibiotics, e.g., ansamycins, polymycins, carbacephem, chloramphenicol, lipopeptide, and drugs against mycobacteria (e.g., the ones causing diseases in mammals, including tuberculosis (Mycobacterium tuberculosis) and leprosy (Mycobacterium leprae), and any combinations thereof. Additional exemplary antimicrobial agent can include, but are not limited to, antibacterial agents, antifungal agents, antiprotozoal agents, antiviral agents, and any mixtures thereof.

In some embodiments of any one of the aspects described herein the anti-microbial agent is an anti-bacterial agent. Exemplary antibacterial agents include, but are not limited to, Acrosoxacin, Amifloxacin, Amoxycillin, Ampicillin, Aspoxicillin, Azidocillin, Azithromycin, Aztreonam, Balofloxacin, Benzylpenicillin, Biapenem, Brodimoprim, Cefaclor, Cefadroxil, Cefatrizine, Cefcapene, Cefdinir, Cefetamet, Cefmetazole, Cefprozil, Cefroxadine, Ceftibuten, Cefuroxime, Cephalexin, Cephalonium, Cephaloridine, Cephamandole, Cephazolin, Cephradine, Chlorquinaldol, Chlortetracycline, Ciclacillin, Cinoxacin, Ciprofloxacin, Clarithromycin, Clavulanic Acid, Clindamycin, Clofazimine, Cloxacillin, Danofloxacin, Dapsone, Demeclocycline, Dicloxacillin, Difloxacin, Doxycycline, Enoxacin, Enrofloxacin, Erythromycin, Fleroxacin, Flomoxef, Flucloxacillin, Flumequine, Fosfomycin, Isoniazid, Levofloxacin, Mandelic Acid, Mecillinam, Metronidazole, Minocycline, Mupirocin, Nadifloxacin, Nalidixic Acid, Nifuirtoinol, Nitrofurantoin, Nitroxoline, Norfloxacin, Ofloxacin, Oxytetracycline, Panipenem, Pefloxacin, Phenoxymethylpenicillin, Pipemidic Acid, Piromidic Acid, Pivampicillin, Pivmecillinam, Prulifloxacin, Rufloxacin, Sparfloxacin, Sulbactam, Sulfabenzamide, Sulfacytine, Sulfametopyrazine, Sulphacetamide, Sulphadiazine, Sulphadimidine, Sulphamethizole, Sulphamethoxazole, Sulphanilamide, Sulphasomidine, Sulphathiazole, Temafioxacin, Tetracycline, Tetroxoprim, Tinidazole, Tosufloxacin, Trimethoprim, and phramceutically acceptable salts or esters thereof.

In some embodiments of any one of the aspects described herein, the anti-bacterial agent is selected from the group consisting of macrolides or ketolides such as erythromycin, azithromycin, clarithromycin, and telithromycin; beta-lactams including penicillin, cephalosporin, and carbapenems such as carbapenem, imipenem, and meropenem; monolactams such as penicillin G, penicillin V, methicillin, oxacillin, cloxacillin, dicloxacillin, nafcillin, ampicillin, amoxicillin, carbenicillin, ticarcillin, meziocillin, piperacillin, azlocillin, temocillin, cepalothin, cephapirin, cephradine, cephaloridine, cefazolin, cefamandole, cefuroxime, cephalexin, cefprozil, cefaclor, loracarbef, cefoxitin, cefmetazole, cefotaxime, ceftizoxime, cefiriaxone, cefoperazone, ceftazidime, cefixime, cefpodoxime, ceftibuten, cefdinir, cefpirome, cefepime, cefadroxil, ceftriaxone, ceftobiprole and astreonam; quinolones such as nalidixic acid, oxolinic acid, norfloxacin, pefloxacin, enoxacin, ofloxacin, levofloxacin, ciprofloxacin, temafloxacin, lomefloxacin, fleroxacin, grepafloxacin, sparfloxacin, trovafloxacin, clinafloxacin, gatifloxacin, moxifloxacin, sitafloxacin, ganefloxacin, gemifloxacin and pazufloxacin; antibacterial sulfonamides and antibacterial sulphanilamides, including para-aminobenzoic acid, sulfadiazine, sulfisoxazole, sulfamethoxazole and sulfathalidine; aminoglycosides such as streptomycin, neomvcin, kanamycin, paromycin, gentamicin, tobramycin, amikacin, netilmicin, spectinomycin, sisomicin, dibekalin and isepamicin; tetracyclines such as tetracycline, chlortetracycline, demeclocycline, minocycline, oxytetracycline, methacycline, doxycycline; rifamycins such as rifampicin (also called rifampin), rifapentine, rifabutin, bezoxazinorifamycin and rifaximin; lincosamides such as lincomycin and clindamycin; glycopeptides such as vancomycin and teicoplanin; streptogramins such as quinupristin and daflopristin; oxazolidinones such as linezolid; polymyxin, colistin and colymycin; trimethoprim, bacitracin, and phosphonomycin. In some preferred embodiments, the antibacterial agent is gentamicin, ampicillin, vancomycin, ceftriaxone or cefepime.

In some embodiments of any one of the aspects described herein the anti-microbial agent is an antifungal agent. Exemplary antifungal agents include, but are not limited to, Bifonazole, Butoconazole, Chlordantoin, Chlorphenesin, Ciclopirox Olamine, Clotrimazole, Eberconazole, Econazole, Fluconazole, Flutrimazole, Isoconazole, Itraconazole, Ketoconazole, Miconazole, Nifuroxime, Tioconazole, Terconazole, Undecenoic Acid, and pharmaceutically acceptable salts or esters thereof. In some embodiments of any one of the aspects described herien, the antifungal agent is selected from the group consisting of azoles (e.g., barleyconazole, butoconazole, clortrimazole, econazole, fluconazole, isavuconazole, itraconazole, ketoconazole, miconazole, oxyconazole, posaconazole, ravuconazole, saperconazole, sulconazole, tercocnazole, tioconazole, voriconazole, and ciclopirox), polyenes (e.g., natamycin, lucensomycin, nystatin, amphotericin B, etc.), echinocandins (e.g., Cancidas), pradimicins (e.g., beanomicins, nikkomycins, sordarins, allylamines, etc.), Triclosan, Piroctone, fenpropimorph, terbinafine, cyclopyroxolamine, flucitocin, griseofulvin haloprozin, tolnaftate, naphthypine, hydrochloride, morpholine, butenapin, undecylenic acid, propionic acid, and derivatives and analogs thereof.

In some embodiments of any one of the aspects described herein, the anti-microbial agent is an antiprotozoal agent. Exemplary antiprotozoal agents include, but are not limited to, Acetarsol, Azanidazole, Chloroquine, Metronidazole, Nifuratel, Nimorazole, Omidazole, Propenidazole, Secnidazole, Sineflngin, Tenonitrozole, Temidazole, Tinidazole, and pharmaceutically acceptable salts or esters thereof.

In some embodiments of any one of the aspects described herein, the anti-microbial agent is an antiviral agent. Exemplary antiviral agents include, but are not limited to, Acyclovir, Brivudine, Cidofovir, Curcumin, Desciclovir, 1-Docosanol, Edoxudine, Fameyclovir, Fiacitabine, Ibacitabine, Imiquimod, Lamivudine, Penciclovir, Valacyclovir, Valganciclovir, and pharmaceutically acceptable salts or esters thereof.

An antimicrobial agent can be, for example, but not limited to, a small molecule, a peptide, a peptidomimetics, an antibody or a fragment thereof, a nucleic acid, an enzyme (e.g., an antimicrobial metalloendopeptidase such as lysostaphin), an aptamer, a drug, an antibiotic, a chemical or any entity that can inhibit the growth and/or kill a microbe. In some embodiments of the any one of the aspects described herein, the anti-microbial agent is an antimicrobial peptide or polymer. Examples of antimicrobial peptides include, but are not limited to, mefloquine, venturicidin A, antimycin, myxothiazol, stigmatellin, diuron, iodoacetamide, potassium tellurite hydrate, aDL-vinylglycine, N-ethylmaleimide, L-allyglycine, diaryquinoline, betaine aldehyde chloride, acivcin, psicofuraine, buthionine sulfoximine, diaminopemelic acid, 4-phospho-D-erythronhydroxamic acid, motexafin gadolinium, xycitrin, cathelicidins, defensins, protegrins, mastoparan, poneratoxin, cecropin, moricin, melittin, magainin, dermaseptin, and/or nisin, or modified versions or analogues thereof.

In some embodiments, the anti-microbial agent is a metal particle. Exemplary metal particles can include silver, titanium oxide, or copper present in any form, e.g., a nanoparticle, a colloid, a suspension, powder, and any combinations thereof. In some embodiments of any one of the aspects described herein the anti-microbial agent is titanium oxide, copper, or silver nanoparticles.

It is noted that compositions and antifouling layers described herein can comprise two or more different antimicrobial agents. Accordingly, in some embodiments of any one of the aspects described herein, the composition or antifouling layer described herein comprises more than one (e.g., at least 2, at least 3, at least 4, at least 5, at least 6, at least 7, at least 8, at least 9, at least 10, or more) different anti-microbial agents. The different anti-microbial agents can be for the same indication, e.g., anti-bacterial, or for different indications, e.g., one anti-microbial agent in the mixture is an anti-bacterial agent and another anti-microbial agent in the mixture is an antifungal agent.

The anti-microbial agent can comprise a functional group for cross-linking. Exemplary functional groups amenable to cross-linking include, but are not limited to, amino, hydroxyl, alkoxy, carbonyl, carboxyl, silyl, silyloxy, hydrocarbyl, sulfhydryl, cycloalkyl, aryl, thio, mercapto, imino, halo, cyano, nitro, azido, sulfoxy, phosphoryl, oxy, quinone, catechol, and the like. In some embodiments of any one of the aspects described herein, the anti-microbial agent comprises at least one amino (NH₂) group.

In some embodiments of any one of the aspects described herein, the anti-microbial agent is covalently linked to the proteinaceous material or the conductive element. For example, the anti-microbial agent is linked to the proteinaceous material or the conductive element via a linker or cross-linker. In some embodiments of any one of the aspects described herein, the anti-microbial agent is covalently linked to the proteinaceous material via a cross-linking agent such as genipin, polyethylene glycol, glutaraldehyde, or carbodiimide cross-linkers. In some preferred embodiments, the anti-microbial agent is covalently linked to the proteinaceous material via genipin.

In some embodiments of any one of the aspects described herein, the anti-microbial agent is covalently linked to the conductive element via a cross-linking agent such as genipin, polyethylene glycol, glutaraldehyde, or and carbodiimide cross-linker. In some preferred embodiments, the anti-microbial agent is covalently linked to the conductive element via genipin

The ratio of the anti-microbial agent to the cross-linking agent can be from about 100:1 to about 1:1 (w/w). In some embodiments of any one of the aspects described herein, the ratio of the anti-microbial agent to the cross-linker is from about 100:1 to about 10:1 (w/w). For example, the ratio of the anti-microbial agent to the cross-linker can be from about 90:1 to about 20:1 w/w, about 80:1 to about 30:1 w/w, about 70:1 to about 40:1 w/w, or about 60:1 to about 50:1 w/w. In some embodiments, the w/w ratio of anti-microbial agent to cross-linker is about 100:1, or about 95:1, or about 90:1, or about 85:1, or about 80:1, about 75:1, or about 70:1, or about 65:1, or about 60:1, about 55:1, or about 50:1, or about 45:1, or about 40:1, about 35:1, or about 30:1, or about 25:1, or about 20:1, about 15:1, or about 10:1.

Similarly, the ratio of the anti-microbial agent to the proteinaceous material in the composition or the antifouling coating layer can be from about 30:1 to about 1:1 (w/w). In some embodiments of any one of the aspects described herein, the ratio of the anti-microbial agent to the proteinaceous material in the composition or the antifouling coating layer is from about 25:1 to about 2:1 w/w, about 20:1 to about 3:1 w/w, about 15:1 to about 4:1 w/w, or about 10:1 to about 5:1 w/w. In some embodiments, the w/w ratio of anti-microbial agent to proteinaceous material in the composition or the antifouling coating layer is about 30:1, or about 29.5:1, or about 29:1, or about 28.5:1, or about 28:1, or about 27.5:1, or about 27:1, or about 26.5:1, or about 26:1, or about 25.5:1, or about 25:1, or about 24.5:1, or about 24:1, or about 23.5:1, or about 23:1, or about 22.5:1, or about 22:1, or about 21.5:1, or about 21:1, or about 20.5:1, or about 20:1, or about 19.5:1, or about 19:1, or about 18.5:1, or about 18:1, or about 17.5:1, or about 17:1, or about 16.5:1, or about 16:1, or about 15.5:1, or about 15:1, or about 14.5:1, or about 14:1, or about 13.5:1, or about 13:1, or about 12.5:1, or about 12:1, or about 11.5:1, or about 11:1, or about 10.5:1, or about 10:1, or about 9.5:1, or about 9:1, or about 8.5:1, or about 8:1, or about 7.5:1, or about 7:1, or about 6.5:1, or about 2.5:1, or about 2:1, or about 1.5:1, or about 1:1.

The ratio of the anti-microbial agent to the conductive element in the composition or the antifouling coating layer can be from about 20:1 to 1:1 (w/w). In some embodiments of any one of the aspects described herein, the ratio of the anti-microbial agent to the conductive element in the composition or the antifouling coating layer is from about 20:1 to about 5:1. For example, the ratio of the anti-microbial agent to the conductive element in the composition or the antifouling coating layer can be from about 15:1 to 5:1 w/w, 12.5:1 to 3:1 w/w, or 5:1 to 4:1 w/w. In some embodiments, the w/w ratio of anti-microbial agent to the conductive element in the composition or the antifouling coating layer is about 20:1, or about 19.5:1, or about 19:1, or about 18.5:1, or about 18:1, or about 17.5:1, or about 17:1, or about 16.5:1, or about 16:1, or about 15.5:1, or about 15:1, or about 14.5:1, or about 14:1, or about 13.5:1, or about 13:1, or about 12.5:1, or about 12:1, or about 11.5:1, or about 11:1, or about 10.5:1, or about 10:1, or about 9.5:1, or about 9:1, or about 8.5:1, or about 8:1, or about 7.5:1, or about 7:1, or about 6.5:1, or about 6:1, or about 5.5:1, or about 5:1

The amount of the antimicrobial agent in the composition or the antifouling coating layer can range from about 1 μg/μL to about 100 μg/μL. For example, the amount of the antimicrobial agent in the composition or the antifouling coating layer can be about 1 μg/μL, about 5 μg/μL, about 10 μg/μL, about 15 μg/μL, about 20 μg/μL, about 25 μg/μL, about 30 μg/μL, about 35 μg/μL, about 40 μg/μL, about 45 μg/μL, about 50 μg/μL, about 55 μg/μL, about 60 μg/μL, about 65 μg/μL, about 70 μg/μL, about 75 μg/μL, about 80 μg/μL, about 85 μg/μL, about 90 ug/μL, about 95 μg/μL, or about 100 μg/μL. In some embodiments, the amount of the antimicrobial agent in the composition or the antifouling coating layer can range from about 1 ug/μL to about 50 μg/μL. For example, the amount of the antimicrobial agent in the composition or the antifouling coating layer can range from about 1 μg/μL to about 50 μg/μL, from about 5 ug/μL to about 25 μg/μL, or from about 10 μg/μL to about 20 μg/μL.

The amount of the antimicrobial agent in the composition or the antifouling coating layer can range from about 0.1% to about 10% (w/v, w/w, or w/v). For example, the amount of the antimicrobial agent in the composition or the antifouling coating layer can be from about 0.15% to about 5%, from about 0.25% to about 2.5%, from about 0.5% to about 2%, or from about 0.75% to about 1.5% (w/v, w/w, or v/v). In some embodiments, amount of the antimicrobial agent in the composition or the antifouling coating layer is about 0.1% about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, about 1% about 1.15%, about 1.2%, about 1.25%, about 1.3%, about 1.35%, about 1.4%, about 1.45%, about 1.5%, about 1.55%, about 1.6%, about 1.65%, about 1.7%, about 1.75%, about 1.8%, about 1.85%, about 1.9%, about 1.95%, or about 2% (w/v, w/w or v/v).

In some embodiments, the composition or the antifouling coating layer further comprises one or more polymers. Exemplary polymers include, but are not limited to, polyethylene glycol, alginate, polytetrafluoroethylene (PTFE), polyacetylene (PA), polyaniline (PANI), polypyrrole (PPy), polythiophene (PTH), poly(para-phenylene) (PPP), poly(phenylenevinylene) (PPV), and polyfuran (PF). In another example, in some implementations, the polymer is a natural polymer such as cellulose, natural silk, cotton, or natural rubbers. In some implementations, the polymer is a synthetic polymer, such as nylon, epoxies, polyethylene (e.g. HDPE and LDPE), polypropylene, polybutadiene, polyethylene terephthalate (PET), polycarbonate, polyurethane, fluorinated polymers (e.g. TEFLON®), polystyrene (e.g. Styrofoam), sulfonated polystyrene, aramide (e.g. KEVLAR®), poly acrylonitrile, poly vinyl acetate, poly vinyl chloride (PVC), poly methyl methacrylate (PMMA), Polyhydroxyethylmethacrylate (PolyHEMA), poly ethers, poly lactic acid, and copolymers and blends of these. In some implementations, the polymer is an ionic polymer, such as a cationic or anionic polymer. In some embodiments, the mixture comprises a polymer selected from the group consisting of polyethylene glycol, alginate, polytetrafluoroethylene (PTFE), polyacetylene (PA), polyaniline (PANI), polypyrrole (PPy), polythiophene (PTH), poly(para-phenylene) (PPP), poly(phenylenevinylene) (PPV), and polyfuran (PF).

In some embodiments, the composition or the antifouling coating layer further comprises a drug eluting component. For example, the composition or the antifouling coating layer further comprises a therapeutic in addition to the anti-microbial agent. Exemplary therapeutic agents include, but are not limited to, anti-inflammatory agents, anti-cancer agents, anti-proliferatives, anti-migratory agents, antifibrotic agents, proapoptotics, anti-neoplastics, immuno-suppressants, and hormones.

In some embodiments, the therapeutic agent is an anti-inflammatory agent. As used herein the term “anti-inflammatory agent” refers to a compound (including its analogs, derivatives, prodrugs and pharmaceutically salts) which can be used to treat inflammation or an inflammation related disease or disorder. Exemplary anti-inflammatory agents include, but are not limited to, the known steroidal anti-inflammatory and non-steroidal anti-inflammatory drugs (NSAIDs). Exemplary steroidal anti-inflammatory agents include but are not limited to 21-acetoxypregnenolone, alclometasone, algestone, amcinonide, beclomethasone, betamethasone, budesonide, chloroprednisone, clobetasol, clobetansone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furcate, paramethosone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, and analogues and derivatives thereof. Exemplary nonsteroidal anti-inflammatory agents include but are not limited to COX inhibitors (COX-1 or COX nonspecific inhibitors) and selective COX-2 inhibitors. Exemplary COX inhibitors include but are not limited to salicylic acid derivatives such as aspirin, sodium salicylate, choline magnesium trisalicylate, salicylate, diflunisal, sulfasalazine and olsalazine; para-aminophenol derivatives such as acetaminophen; indole and indene acetic acids such as indomethacin and sulindac; heteroaryl acetic acids such as tolmetin, dicofenac and ketorolac; arylpropionic acids such as ibuprofen, naproxen, flurbiprofen, ketoprofen, fenoprofen and oxaprozin; anthranilic acids (fenamates) such as mefenamic acid and meloxicam; enolic acids such as the oxicams (piroxicam, meloxicam); alkanones such as nabumetone; and analogues and derivatives thereof. Exemplary COX-2 inhibitors include but are not limited to diarylsubstituted furanones such as refecoxib; diaryl-substituted pyrazoles such as celecoxib; indole acetic acids such as etodolac and sulfonanilides such as nimesulide; and analogues and derivatives thereof. Additional anti-inflammatory agents include, but are not limited to sirolimus, everolimus, biolimus (A9), zotarolimus (ABT-578), tacrolimus, pimecrolimus, and genistein.

In some embodiments of any one of the aspects described herein, the composition and/or the antifouling coating layer described herein further comprises one or more anti-inflammatory drugs such as sirolimus, everolimus, biolimus (A9), zotarolimus (ABT-578), tacrolimus, and pimecrolimus, genistein, steroids (dexamethasone, prednisolone, methylprednisolone and hydrocortisone), fluocinolone acetonide, hormones etc.

In some embodiments, the composition or the antifouling coating layer has antimicrobial activity.

### Cross-Linker

The components of the composition and/or the antifouling coating layer, e.g., the proteinaceous material, the conductive element, the redox mediator, the oxidoreductase, and if present, the target binding molecule and the anti-microbial agent can be cross-linked to each other or to themselves. As used herein, the term “cross-linked” is intended to refer to two or more molecules covalently bonded together. Cross-linking can be intermolecular, i.e., between different components/molecules, or intramolecular, e.g., between the same component/molecule. Further, covalent bonding between two cross-linkable components/molecules can be direct, in which case an atom in one component/molecule is directly bound to an atom in the other component/molecule, or it can be indirect, through a linking group/agent. The term “cross-linkable” refers to a component or molecule that is capable of undergoing reaction to form a cross-linked composition.

The terms “cross-linking agent”, “cross-linker”, and the like are used interchangeably herein and refer to a compound or molecule that can create a covalent linkage between two cross-linkable components/molecules. Generally, a cross-linking agent contains at least two reactive functional groups that generate covalent bonds between two or more molecules. Cross-linking agents can be homobifunctional (i.e., having two identical reactive ends) or heterobifunctional (i.e., having two different reactive ends). Suitable cross-linking agents include, but are not limited to, genipin; polyethylene glycol; glutaraldehyde; nordihydroguaiaretic acid (NDGA); 3,4-dihydroxyphenylalanine; 1,2-benzenediol; 2,3-dihydroxynaphthalene; 1,3-benzenediol; adrenalone; catechin; nitrocatechol; 3,4-dihydroxybenzaldehyde; 3,4-dihydroxybenzoic acid; deoxyepinephrine; dobutamine; dopamine; dopexamine; epinephrine; nordefrin; 3-pentadecylcatechol; carbodiimides (e.g., 1-ethyl-3-(3-dimethylaminopropyl) carbodiimide hydrochloride (EDC)); formaldehyde; tannic acid; isocyanates; epichlorohydrin; oxalic acid; malonic acid; succinic acid; glutaric acid; adipic acid; pimelic acid; fumaric acid; maleic acid; malic acid; tartrate; bisepoxides; divinyl sulfone and derivatives (e.g., divinyl sulfone (DVS)); butanediol diglycidyl ether (BDDE); dimethyl adipimidate (DMA), dimethyl suberimidate (DMS), Bissulfosuccinimidyl suberate, formaldehyde, p-azidobenzoyl hydrazide; n-5-azido-2-nitrobenzoyloxysuccinimide; n-[4-(p-azidosalicylamido) butyl]-3′-(2′-pyridyldithio) propionamide; p-azidophenyl glyoxal monohydrate; bis[b-(4-azidosalicylamido)ethyl]disulfide; bis[2-(succinimidooxycarbonyloxy)ethyl]sulfone; 1,4-di [3′-(2′-pyridyldithio) propionamido]butane; dithiobis(succinimidyl propionate); disuccinimidyl suberate; disuccinimidyl tartrate; 3,3′-dithiobis(sulfosuccinimidyl propionate); 3,3′-dithiobis(sulfosuccinimidyl propionate) 1-ethyl-3-[3-dimethylaminopropyl]carbodiimide hydrochloride; ethyleneglycol bis(succinimidyl succinate); N-(E-maleimidocaproic acid hydrazide); [N-(E-maleimidocaproyloxy)-succinimide ester]; N-maleimidobutyryloxysuccinimide ester; hydroxylamine.HCl; maleimide-PEG-succinimidyl carboxy methyl; m-maleimidobenzoyl-N-hydroxysuccinimide Ester; N-hydroxysuccinimidyl-4-azidosalicylic acid; N-(p-maleimidophenyl isocyanate); N-succinimidyl(4-iodoacetyl) aminobenzoate; succinimidyl-4-(N-maleimidomethyl)cyclohexane-1-carboxylate; succinimidyl 4-(p-maleimidophenyl) butyrate; sulfo-disulfosuccinimidyl tartrate; [N-(E-maleimidocaproyloxy)-sulfo succinimide ester; N-Maleimidobutyryloxysulfosuccinimide ester; N-hydroxysulfosuccinimidyl-4-azidobenzoate; m-Maleimidobenzoyl-N-hydroxysulfo succinimide ester; sulfosuccinimidyl(4-azidophenyl)-1,3 dithio propionate; sulfosuccinimidyl 2-(m-azido-o-nitrobenzamido)-ethyl-1,3′-dithio propionate; sulfosuccinimidyl 6-(4′-azido-2′-nitrophenylamino) hexanoate; sulfosuccinimidyl-2-(p-azidosalicylamido)ethyl-1,3-dithiopropionate; N-(Sulfosuccinimidyl (4-iodoacetyl) amino benzoate); sulfosuccinimidyl-4-(N-maleimidomethyl)cyclohexane-1-carboxylate; sulfosuccinimidyl 4-(p-maleimidophenyl) butyrate; poly(ethylene glycol) diglycidyl ether (PEGDE); poly(propylene glycol) diglycidyl ether (PPGDE); and the like. In some embodiments of any one of the aspects described herein, the cross-linking agent is genipin, polyethylene glycol, glutaraldehyde, or carbodiimide cross-linkers. In some preferred embodiments of any one of the aspects described herein the cross-linking agent is genipin.

### Antifouling Surface

Without wishing to be bound by a theory, the composition, i.e., the composition described herein has antifouling properties. As used herein, the term “antifouling” refers to the effect of preventing, reducing and/or eliminating fouling, i.e., preventing, reducing and/or eliminating the aggregation of molecules such as biomolecules on a surface such that the surface maintains its initial physical and/or chemical properties (e.g. conductivity). Thus, in some embodiments, the composition is antifouling. The composition can be coated on a surface to impart antifouling properties to the surface. Accordingly, in another aspect provided herein is a surface with antifouling properties. For example, the surface with the antifouling properties comprises a composition described herein.

In the context of this specification, the term “coated” means that a layer of is present on a surface. For example, a layer of antifouling layer on a surface or a layer of probe on the antifouling layer. The amount of the probe used to coat the antifouling layer can vary with a number of factors such as surface area, coating density, types of probe, and binding performance.

Without wishing to be bound by a theory, the composition described herein is biocompatible. The term “biocompatible” refers to a material's ability to perform its intended function, with a desired degree of incorporation in a host, without eliciting any undesirable local or systemic effects in that host. Accordingly, in some embodiments, the coated surface is a surface of a medical device. As used herein, a “medical device” refers to a non-naturally occurring object that is inserted or implanted in a subject or applied to a surface of a subject. Exemplary medical devices include, but are not limited to, fibers (wound dressings, bandages, gauze, tape, pads, sponges, including woven and non-woven sponges and those designed specifically for dental or ophthalmic surgeries), surgical, medical or dental instruments, blood oxygenators, ventilators, pumps, drug delivery devices, tubing, wiring, electrodes, contraceptive devices, feminine hygiene products, endoscopes, grafts (including small diameter <6 mm), stents (including coronary, uretheral, renal, biliary, colorectal, esophageal, pulmonary, urethral, and vascular), stent grafts (including abdominal, thoracic, and peripheral vascular), pacemakers, implantable cardioverter-defibrillators, cardiac resynchronization therapy devices, cardiovascular device leads, ventricular assist devices and drivelines, heart valves, vena cava filters, endovascular coils, catheters (including central venous, peripheral central, midline, peripheral, tunneled, dialysis access, urinary, neurological, peritoneal, intra-aortic balloon pump, angioplasty balloon, diagnostic, interventional, drug delivery, etc.), catheter connectors and valves (including needleless connectors), intravenous delivery lines and manifolds, shunts, wound drains (internal or external including ventricular, ventriculoperitoneal, and lumboperitoneal), dialysis membranes, infusion ports, cochlear implants, endotracheal tubes, tracheostomy tubes, ventilator breathing tubes and circuits, guide wires, fluid collection bags, drug delivery bags and tubing, implantable sensors (e.g., intravascular, transdermal, intracranial), ophthalmic devices including contact lenses, orthopedic devices (including hip implants, knee implants, shoulder implants, spinal implants (including cervical plates systems, pedicle screw systems, interbody fusion devices, artificial disks, and other motion preservation devices), screws, plates, rivets, rods, intramedullary nails, bone cements, artificial tendons, and other prosthetics or fracture repair devices), dental implants, periodontal implants, breast implants, penile implants, maxillofacial implants, cosmetic implants, valves, appliances, scaffolding, suturing material, needles, hernia repair meshes, tension-free vaginal tape and vaginal slings, prosthetic neurological devices, tissue regeneration or cell culture devices, or other medical devices used within or in contact with the body or any portion of any of these.

In some embodiments, the antifouling coating layer is directly or indirectly connected to an electrode.

### Electrode

Accordingly, in another aspect, provided herein is an electrode comprising a conductive substrate and an antifouling coating layer on at least a portion of a surface of the conductive substrate, wherein the antifouling coating layer comprises a composition described herein. For example, the electrode comprises: (i) a conductive substrate (e.g., an electrically conductive substrate); and (2) an antifouling coating layer on at least a portion of a surface of the conductive substrate, and wherein the antifouling coating layer comprises: a proteinaceous material, a conductive element, a redox mediator, and an oxidoreductase. Optionally, the redox mediator is covalently linked with the proteinaceous material, the conductive element, or the surface coated by the antifouling coating layer. Optionally, the oxidoreductase is covalently linked with the proteinaceous material, the conductive element, or the surface coated by the antifouling coating layer.

As used herein an “electrode” is a conductor through which current enters or leaves a medium, where the medium is nonmetallic (i.e., it emits or collects electrons or electron “holes”). For example, the medium can be a complex matrix (e.g., blood or serum). The electrode can be inserted into/onto a tissue such as mammalian tissue and be contacted with tissue and/or fluids therein/thereon. The electrode can be large (e.g., with a working surface area of greater than 1 cm², greater than 10 cm², greater than 100 cm²) or the electrode can be small (e.g., with a working surface area of less than 1 cm², less than 1 mm², less than 100 μm², less than 10 μm², less than 1 μm²). The working surface area is the area in contact with the medium and wherein current enters or leaves the medium.

The conductive substrate can be in any form having a surface that can be coated. For example, the conductive substrate can be included in the form of a conductive particle, a conductive nano-particle, a conductive micro-particle, a conductive nano-fiber, a conductive micro-fiber, a conductive flake, a conductive chip, a conductive crystal, a conductive porous substrate, a conductive wafer, a conductive wire, a conductive nano-wire, a conductive micro-wire, a conductive channel, a conductive nano-channel, a conductive micro-channel, a conductive rod, a conductive nano-rod, a conductive micro-rod, a conductive foil, a conductive sheet, a conductive web, or combination of these forms. In some implementations, the conductive substrate is part of a microfluidic device, such as a channel or chamber therein.

Metal patterning techniques, such as standard printed circuit board (PCB) technology, offer a number of versatile fabrication options such as (i) track size and spacing less than 100 μm; (ii) high purity electrolytic gold plating several microns thick suitable for electrochemistry and surface modification chemistries; (iii) ease of small scale prototyping in standard laboratory settings; and (iv) large scale mass manufacturing capabilities at a fraction of the cost of high-end microarrays. In some embodiments, electrodes as disclosed herein may be fabricated using PCB technology.

In some embodiments, the electrodes are mass fabricated onto non-electrically conductive surfaces such as plastic substrates using inexpensive standard technology such as printed circuit board (PCB) technology, roll-to-roll laser ablation or evaporation. Exemplary non-electrically conductive surfaces include plastic, poly(carbonate) (PC), poly(methyl methacrylate) (PMMA), cyclic olefin polymers (COP) or cyclic olefin copolymers (COC), SU-8, parylene, silicon nitride, kapton, styrene-ethylene-butylene-styrene (SEBS), poly-dimethysiloxane (PDMS), polyimide, silicon dioxide, and any combination thereof.

In some embodiments, the electrode is a planar or a 3-dimensional electrode. As used herein, a planar electrode electrically interacts with an electroactive species or mediator on a 2-dimensional surface. As used herein, a 3-dimensional electrode is an electrode displaying a very high surface area per unit volume, caused by no planarity. Without being bound by theory, this provides high turbulence at their interface with an electroactive species or mediator, enhancing the mass transfer process of the electroactive species towards the electrode surface. These characteristics strongly improve the electrochemical reaction rate.

In some embodiments the electrode is “Multiplexed” such that it is configured for a multiplexed assay. As used herein a “multiplexed” assay can be used to simultaneously measure multiple analytes or signals such as two or more (e.g., 3 or more, 5 or more, 10 or more, 50 or more, 100 or more, 1000 or more) during a single run or cycle of the assay. The electrode can therefore be configured as an array of electrodes, microelectrodes or electrochemical sensors each of which can be independently electrically attached to a circuit for monitoring the electrical signals. For example, the array of electrodes can be disposed at the bottom, sides or top of a multiwell plate (e.g., microwell plate) arrayed on a flat surface such as a semiconductor chip (e.g., a sensor array chip) or form part of a multielectrode array (e.g., for connection of neurons to electronic circuitry). In some embodiments, the compositions as described herein, can coat more than one sensor since the coating will not conduct between the sensors due to the anisotropy of the conduction, therefore an array of conductors, sensors or electrodes can be coated forming a multiplexed electrode.

Electrodes can include materials with metallic conduction and semiconductors. For example, electrodes can include metals, metal alloys, semiconductors, doped materials, conducting ceramics and conducting polymers. Without limitation, electrode materials can include carbon (e.g., graphite, glassy carbon, conductive polymers), copper, titanium, brass, mercury, silver, platinum, palladium, gold, rhodium, zinc, lead, tin, iron, Indium Tin Oxide (ITO), aluminum, stainless steel, tungsten, nickel, zirconium, niobium, tantalum, chromium, molybdenum, manganese, rhenium, ruthenium, rhodium, iridium, silicon, doped silicon, II-VI semiconductors (e.g., ZnO, ZnS, CdSe), III-V semiconductors such as (e.g., GaAs, InSb), ceramics (e.g., TiO₂, Fe₃O₄, MgCr₂O₄), and conductive polymers (e.g., poly(acetylene)s, poly(p-phenylene vinylene), poly(fluorenes)s, polyphenylenes, polypyrenes, polyazulenes, polynaphthalenes, polyanilines, polyazepines, polyindoles, polycarbazoles, poly(pyrrole)s, poly(thiophene)s, poly(3,4-ethylenedioxythiophene)), polyimide, parylene, benzocyclobutene, and combinations, mixtures and alloys of these.

In some embodiments, the conductive substrate includes a metal, a metalloid, a conducting polymer, a conducting glassy material, a conducting amorphous material, a conducting biological membrane, a conducting carbon-based material, or any combination of these.

In some embodiments, the conductive substrate includes gold. In some embodiments, the conductive substrate includes a silica-based glass (e.g., pure silica or mixtures such as borosilicate glass). In some embodiments, the conductive substrate includes graphite, diamond, glassy carbon, or carbon nano-tubes (CNTs). In some implementations, the conductive substrate is a chip including gold and a silica-based glass.

In some embodiments of any one of the aspects described herein, the conductive substrate is a flexible substrate. For example, the conductive substrate comprises a flexible material. Exemplary materials for the flexible substrate include, but are not limited to, polyethylene terephthalate, polyethylene naphathalate, polyimides, polymeric hydrocarbons, celluloses, plastics, polycarbonates, polystyrenes, and any combination thereof.

Electrodes can also include insulating components such as insulators for electrical and mechanical protection, imparting rigidity and electrical isolation to parts of the electrode.

In some embodiments, electrode can be a carbon-based electrode.

In some embodiments, the electrode can be part of an electrochemical cell. For example, the electrode is a working electrode and the electrochemical cell can include a counter electrode and reference electrode.

Electrochemical methods are methods that rely on a change in the potential, charge or current to characterize the analyte's chemical reactivity. Some examples include potentiometry, controlled current coulometry, controlled-potential coulometry, amperometry, stripping voltammetry, hydrodynamic voltammetry, polarography, stationary electrode voltammetry, pulsed polarography, electrochemical impedance spectroscopy and cyclic voltammetry. The signals are detected using an electrode or electrochemical sensors coupled to circuits and systems for collection, manipulation and analysis of the signals.

In some embodiments, the antifouling coating layer of the electrode is adapted for contact with an analyte or a sample comprising an analyte. The antifouling coating can allow analyte to flow through the pores and be detected, for example, by binding to a capture molecule such as an antibody, DNA strand, or aptamer. In some implementations, the coatings can be patterned as a conductive wire or a dielectric/insulating surface. Some implementations include coating microfluidic chips, lab-on-a-chip, and organs on a chip. In some implementations, the coatings can be used in nano-gap and micro-gap devices. For example, these devices include nano-gap electrodes, nanostructured-based electrical biosensors, and nano-gap dielectric biosensor for label free DNA hybridization detection. The coatings can be applied, for example, to the gap between electrodes in the device and thereby protect the surfaces of the gap from fouling. In some implementations, the coating is a cross-linked and porous gel, and the gap is completely or mostly filled in. Analyte can flow through the pores and be detected, for example, by binding to a capture molecule such as an antibody, DNA strand, or aptamer.

### Antifouling Coating Layer

Embodiments of the various aspects described herein include an antifouling coating layer. Generally, the antifouling coating layer comprises a composition described herein. For example, the antifouling coating layer comprises: a proteinaceous material, a conductive element, a redox mediator, and an oxidoreductase. Optionally, the redox mediator is covalently linked with the proteinaceous material, the conductive element, or the surface coated by the antifouling coating layer. Optionally, the oxidoreductase is covalently linked with the proteinaceous material, the conductive element, or the surface coated by the antifouling coating layer.

In some embodiments of any one of the aspects described herein, the antifouling coating layer is porous. For example, the antifouling coating layer comprises macropores. As used herein, the term “macropore” means pores whose aperture, width or diameter is greater than 100 nm. In some embodiments of any one of the aspects described herein, macropores have an aperture, width or diameter from about 0.1 μm to about 10 μm. For example, macropores have an aperture, width or diameter from about 0.25 μm to about 7.5 μm, from about 0.5 μm to about 5 μm, from about 0.75 μm to about 2.5 μm, or from about 1 μm to about 3 μm. In some embodiments of any one of the aspects described herein, macropores have an aperture, width or diameter of about 0.1 μm, about 0.15 μm, about 0.2 μm, about 0.25 μm, about 0.3 μm, about 0.35 μm, about 0.4 μm, about 0.45 μm, about 0.5 μm, about 0.55 μm, about 0.6 μm, about 0.65 μm, about 0.7 μm, about 0.75 μm, about 0.8 μm, about 0.85 μm, about 0.9 μm, about 0.95 μm, about 1 μm, about 1.05 μm, about 1.1 μm, about 1.15 μm, about 1.2 μm, about 1.25 μm, about 1.3 μm, about 1.35 μm, about 1.4 μm, about 1.45 μm, about 1.5 μm, about 1.55 μm, about 1.6 μm, about 1.65 μm, about 1.7 μm, about 1.75 μm, about 1.8 μm, about 1.85 μm, about 1.9 μm, about 1.95 μm, about 2 μm, about 2.05 μm, about 2.1 μm, about 2.15 μm, about 2.2 μm, about 2.25 μm, about 2.3 μm, about 2.35 μm, about 2.4 μm, about 2.45 μm, about 2.5 μm, about 2.55 μm, about 2.6 μm, about 2.65 μm, about 2.7 μm, about 2.75 μm, about 2.8 μm, about 2.85 μm, about 2.9 μm, about 2.95 μm, about 3 μm, about 3.05 μm, about 3.1 μm, about 3.15 μm, about 3.2 μm, about 3.25 μm, about 3.3 μm, about 3.35 μm, about 3.4 μm, about 3.45 μm, about 3.5 μm, about 3.55 μm, about 3.6 μm, about 3.65 μm, about 3.7 μm, about 3.75 μm, about 3.8 μm, about 3.85 μm, about 3.9 μm, about 3.95 μm, about 4 μm, about 4.05 μm, about 4.1 μm, about 4.15 μm, about 4.2 μm, about 4.25 μm, about 4.3 μm, about 4.35 μm, about 4.4 μm, about 4.45 μm, about 4.5 μm, about 4.55 μm, about 4.6 μm, about 4.65 μm, about 4.7 μm, about 4.75 μm, about 4.8 μm, about 4.85 μm, about 4.9 μm, about 4.95 μm, or about 5 μm.

In some embodiments of any one of the aspects described herein, the antifouling coating layer comprises mesopores. As used herein, the term “mesopores” means pores whose aperture, width or diameter is between about 5 nm and about 99 nm. In some embodiments of any one of the aspects described herein, mesopores have an aperture, width or diameter from about 5 nm to about 50 nm. For example, mesopores have an aperture, width or diameter of about 5 nm, about 5.5 nm, about 6 nm, about 6.5 nm, about 7 nm, about 7.5 nm, about 8 nm, about 8.5 nm, about 9 nm, about 9.5 nm, about 10 nm, about 10.5 nm, about 11 nm, about 11.5 nm, about 12 nm, about 12.5 nm, about 13 nm, about 13.5 nm, about 14 nm, about 14.5 nm, about 15 nm, about 15.5 nm, about 16 nm, about 16.5 nm, about 17 nm, about 17.5 nm, about 18 nm, about 18.5 nm, about 19 nm, about 19.5 nm, about 20 nm, about 20.5 nm, about 21 nm, about 21.5 nm, about 22 nm, about 22.5 nm, about 23 nm, about 23.5 nm, about 24 nm, about 24.5 nm, about 25 nm, about 25.5 nm, about 26 nm, about 26.5 nm, about 27 nm, about 27.5 nm, about 28 nm, about 28.5 nm, about 29 nm, about 29.5 nm, about 30 nm, about 30.5 nm, about 31 nm, about 31.5 nm, about 32 nm, about 32.5 nm, about 33 nm, about 33.5 nm, about 34 nm, about 34.5 nm, about 35 nm, about 35.5 nm, about 36 nm, about 36.5 nm, about 37 nm, about 37.5 nm, about 38 nm, about 38.5 nm, about 39 nm, about 39.5 nm, about 40 nm, about 40.5 nm, about 41 nm, about 41.5 nm, about 42 nm, about 42.5 nm, about 43 nm, about 43.5 nm, about 44 nm, about 44.5 nm, about 45 nm, about 45.5 nm, about 46 nm, about 46.5 nm, about 47 nm, about 47.5 nm, about 48 nm, about 48.5 nm, about 49 nm, about 49.5 nm, or about 50 nm. In some embodiments of any one of the aspects described herein, the mesopores have an aperture, width or diameter from about 5 nm to about 20 nm. For example, the mesopores have an aperture, width or diameter from about 10 nm to about 15 nm.

In some embodiments of any one of the aspects described herein, the antifouling coating layer comprises macropores and mesopores. For example, the antifouling coating layer comprises macropores having an aperture, width or diameter from about 0.1 μm to about 10 μm, and mesopores having an aperture, width or diameter from about 5 nm to about 50 nm. In some embodiments of any one of the aspects described herein, the antifouling coating layer comprises macropores having an aperture, width or diameter from about 1 μm to about 5 μm, and mesopores having an aperture, width or diameter from about 10 nm to about 15 nm.

In some embodiments of any one of the aspects described herein, the antifouling coating layer comprises nanopores. As used herein, the term “nanopores” means pores whose aperture, width or diameter is less than about 5 nm, typically strictly greater than 0 and less than about 5 nm.

In some embodiments of any one of the aspects described herein, the antifouling coating layer comprises macropores and nanopores.

In some embodiments of any one of the aspects described herein, the antifouling coating layer comprises macropores, mesopores and nanopores.

The antifouling coating layer is porous. As used herein, the term “porous” in the context antifouling coating layer means the antifouling coating layer comprises a plurality of spores, holes, openings, bores, apertures, spaces, perforations, or intervals. While the term porous indicates the presence of voids, it does not specify the specific size of the spores, holes, openings, bores, apertures, spaces, perforations, or intervals. The term “porosity” is widely understood as the ratio of void volume to total volume of a three-dimensional porous body, where the total volume is determined by the macroscopic outer dimensions of the body. Porosity can be indicated as a fraction between 0-1 or as a percentage between 0-100%. Porosity can be measure by instruments in the art, such as a porometer. Porosity is inversely proportional to density of the material. Thus, the porosity also can be determined by measuring the density of the coating layer. In some embodiments of any one of the aspects described herein, the porosity can be determined by mercury porosimetry analysis. In some embodiments, mercury porosimetry analysis corresponds to the intrusion of a volume of mercury characteristic of the existence of pores in the antifouling coating layer according to the ASTM D4284-83 standard.

Generally, the antifouling coating layer has a porosity from about 5% to about 95%. For example, the antifouling coating layer has a porosity from about 10% to about 75%, about 15% to about 70%, about 20% to about 65%, about 25% to about 60%, or about 30% to about 55%. In some embodiments of any one of the aspects described herein, the antifouling coating layer has a porosity from about 35% to about 45%. It is noted porosity can be controlled by altering the ratio of non-aqueous phase to the aqueous phase and/or using different types of materials for the non-aqueous and/or aqueous phases.

The antifouling coating layer can have a thickness greater than about 2 nm. In some embodiments, the antifouling coating layer has a thickness from about 20 nm to about 100 μm, from about 200 nm to about 75 μm, from about 1 μm to about 50 μm, or from about 2 μm to about 25 μm. In some embodiments of any one of the aspects described herein, the antifouling coating layer has a thickness from about 2 nm to about 100 μm, from about 20 nm to about 75 μm, from about 200 nm to about 50 μm, from about 1 μm to about 25 μm, or from about 2 μm to about 10 μm. For example, the antifouling coating layer has a thickness from about 2 nm to about 20 μm. In some embodiments of any one of the aspects described herein, the antifouling coating layer has a thickness of about 2 nm, about 5 nm, about 10 nm, about 15 nm, about 20 nm, about 25 nm, about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 0.125 μm, about 0.15, about 0.175, about, 0.2, about 0.225, about 0.25, about 0.275 μm, about 3 μm, about 0.325 μm, about 0.35 μm, about 0.375 μm, about 4 μm, about 0.425 μm, about 0.45 μm, about 0.475 μm, about 5 μm, about 0.525 μm, about 0.55 μm, about 0.575 μm, about 6 μm, about 0.625 μm, about 0.65 μm, about 0.675 μm, about 7 μm, about 0.725 μm, about 0.75 μm, about 0.775 μm, about 8 μm, about 0.825 μm, about 0.85 μm, about 0.875 μm, about 9 μm, about 0.925 μm, about 0.95 μm, about 0.975 μm, about 1 μm, about 1.25 μm, about 1.5 μm, about 1.75 μm, about 2 μm, about 2.25 μm, about 2.5 μm, about 2.75 μm, about 3 μm, about 3.25 μm, about 3.5 μm, about 3.75 μm, about 4 μm, about 4.25 μm, about 4.5 μm, about 4.75 μm, about 5 μm, about 5.25 μm, about 5.5 μm, about 5.75 μm, about 6 μm, about 6.25 μm, about 6.5 μm, about 6.75 μm, about 7 μm, about 7.25 μm, about 7.5 μm, about 7.75 μm, about 8 μm, about 8.25 μm, about 8.5 μm, about 8.75 μm, about 9 μm, about 9.25 μm, about 9.5 μm, about 9.75 μm, about 10 μm, about 10.25 μm, about 10.5 μm, about 10.75 μm, about 11 μm, about 11.25 μm, about 11.5 μm, about 11.75 μm, about 12 μm, about 12.25 μm, about 12.5 μm, about 12.75 μm, about 13 μm, about 13.25 μm, about 13.5 μm, about 13.75 μm, about 14 μm, about 14.25 μm, about 14.5 μm, about 14.75 μm, about 15 μm, about 15.25 μm, about 15.5 μm, about 15.75 μm, about 16 μm, about 16.25 μm, about 16.5 μm, about 16.75 μm, about 17 μm, about 17.25 μm, about 17.5 μm, about 17.75 μm, about 18 μm, about 18.25 μm, about 18.5 μm, about 18.75 μm, about 19 μm, about 19.25 μm, about 19.5 μm, about 19.75 μm, about 20 μm, about 10.25 μm, about 10.5 μm, about 10.75 μm, about 21 μm, about 21.25 μm, about 21.5 μm, about 21.75 μm, about 22 μm, about 22.25 μm, about 22.5 μm, about 22.75 μm, about 23 μm, about 23.25 μm, about 23.5 μm, about 23.75 μm, about 24 μm, about 24.25 μm, about 24.5 μm, about 24.75 μm, about 25 μm, about 25.25 μm, about 25.5 μm, about 25.75 μm, about 26 μm, about 26.25 μm, about 26.5 μm, about 26.75 μm, about 27 μm, about 27.25 μm, about 27.5 μm, about 27.75 μm, about 28 μm, about 28.25 μm, about 28.5 μm, about 28.75 μm, about 29 μm, about 29.25 μm, about 29.5 μm, about 29.75 μm, about 30 μm, about 30.25 μm, about 30.5 μm, about 30.75 μm, about 31 μm, about 31.35 μm, about 31.5 μm, about 31.75 μm, about 32 μm, about 32.35 μm, about 32.5 μm, about 32.75 μm, about 33 μm, about 33.35 μm, about 33.5 μm, about 33.75 μm, about 34 μm, about 34.35 μm, about 34.5 μm, about 34.75 μm, about 35 μm, about 35.35 μm, about 35.5 μm, about 35.75 μm, about 36 μm, about 36.35 μm, about 36.5 μm, about 36.75 μm, about 37 μm, about 37.35 μm, about 37.5 μm, about 37.75 μm, about 38 μm, about 38.35 μm, about 38.5 μm, about 38.75 μm, about 39 μm, about 39.35 μm, about 39.5 μm, about 39.75 μm, about 40 μm, about 40.35 μm, about 40.5 μm, about 40.75 μm, about 41 μm, about 41.45 μm, about 41.5 μm, about 41.75 μm, about 42 μm, about 42.45 μm, about 42.5 μm, about 42.75 μm, about 43 μm, about 43.45 μm, about 43.5 μm, about 43.75 μm, about 44 μm, about 44.45 μm, about 44.5 μm, about 44.75 μm, about 45 μm, about 45.45 μm, about 45.5 μm, about 45.75 μm, about 46 μm, about 46.45 μm, about 46.5 μm, about 46.75 μm, about 47 μm, about 47.45 μm, about 47.5 μm, about 47.75 μm, about 48 μm, about 48.45 μm, about 48.5 μm, about 48.75 μm, about 49 μm, about 49.45 μm, about 49.5 μm, about 49.75 μm, or about 50 μm. The thickness of antifouling layer can also have a thickness from 100 μm to 500 μm via printing techniques such as screen printing, inkjet printing, and layer-by-layer coating. Residue can be removed by washing with solvent or blowing, which makes films have good uniformity.

### Methods for Preparing the Antifouling Coating Layer

In another aspect provided herein is a method for preparing a surface with an antifouling coating layer. The method comprises coating at least a part of a surface with a composition described herein.

In some embodiments, the method for preparing a surface with an antifouling coating layer comprises a layer-by-layer assembly. For example, the method comprises: (i) forming a first layer comprising a proteinaceous material and a conductive element on at least a part of the surface; (ii) optionally, cross-linking the proteinaceous material and/or the conductive element; (iii) depositing a composition comprising the redox mediator on a surface of the first coating layer; (iv) optionally, covalently linking the redox mediator with the proteinaceous material, the oxidoreductase and/or the surface; (v) depositing a composition comprising the oxidoreductase on the coating layer surface comprising the redox mediator; and (vi) optionally, covalently linking the oxidoreductase with the proteinaceous material, the oxidoreductase and/or the surface.

In some embodiments, the method for preparing a surface with an antifouling coating layer comprises: (i) forming a first layer comprising a proteinaceous material and a conductive element on at least a part of the surface; (ii) optionally, cross-linking the proteinaceous material and/or the conductive element; (iii) depositing a composition comprising the redox mediator on a surface of the first coating layer; (iv) optionally, covalently linking the oxidoreductase with the proteinaceous material, the oxidoreductase and/or the surface; (v) depositing a composition comprising the redox mediator on the coating layer surface comprising the redox mediator; and (vi) optionally, covalently linking the redox mediator with the proteinaceous material, the oxidoreductase and/or the surface.

The surface can be coated by any suitable technique known in the art. Exemplary coating methods include, but are not limited to, spin coating, nozzle-assisted printing (e.g., inkjet printing), drop-casting, blade coating, 3D printing, zone-casting, roll coating, roll-to-roll (R2R) coating, spray coating, dip coating, die coating, slot die coating, roll coating, comma coating gravure coating, bar coating, vapor coating, knife coating, or combinations thereof.

In some embodiments, the surface is coated by spin-coating. Spin coating is a surface coating method in which the coating material, e.g., a composition described herein is deposited on the surface to be coated. The surface is attached to a spinner, which causes the spinner to rotate the surface at a controlled speed, thereby spreading the coating material onto the surface and wetting the surface entirely with the coating material. Generally, the surface to be coated is spun at from about 250 rpm to about 5000 rpm. For example, the surface to be coated is spun at from about 500 rpm to about 4000 rpm, from about 1000 rpm to about 3000 rpm, from about 750 rpm to about 2500 rpm or from about 1000 rpm to about 2000 rpm. Inventors have discovered inter alia spinning the surface at about 1500 rpm unexpectedly provides a highly uniform coating layer. Accordingly, in some embodiments of any one of the aspects described herein, the surface to be coated is spun at from about 1250 rpm to about 1750 rpm. In some preferred embodiments, the surface to be coated is spun at about 1500 rpm

In some embodiments of any one of the aspects described herein, the surface is coated by nozzle-assisted printing (e.g., inkjet printing). Nozzle-assisted printing is a surface coating method in which ink jet technology is used to deposit coating materials on surfaces. Generally, the coating material is injected under pressure (e.g., from about 5 kPa to about 20 kPa). The printing speed can be adjusted as needed to make a uniform coating layer on the surface. For example, the printing speed can be from about 5 mm/s to about 20 mm/s. The temperature of printer bed can be an elevated temperature, e.g., a temperature of about 37° C. or higher. In some embodiments, the temperature of the printer bed can be from about 37° C. to about 70° C., from about 40° C. to about 60° C., or from about 45° C. to about 55° C. In some embodiments, the temperature of the printer bed is about 50° C.

In some embodiments of any one of the aspects described herein, the surface is coated by dip coating. Dip coating is a surface coating method in which the surface to be treated is immersed and then withdrawn from the coating material, e.g., a composition described herein at a defined rate. The dip coating process can be, generally, separated into 3 stages: (i) immersion: the surface is immersed in the solution of the coating material at a constant speed; (ii) dwell time: the surface remains fully immersed and motionless to allow for the coating material to apply itself to the surface; and (iii) withdrawal: the surface is withdrawn, again at a constant speed. The faster the substrate is withdrawn the thicker the coating material that will be applied to the surface.

In some embodiments, the method for preparing a surface with an antifouling coating layer comprises a step of cross-linking the proteinaceous material. As used herein, the term “crosslinking” refers to the formation of a bond between the same molecule (e.g., the same proteinaceous material molecule) or between different molecules (e.g., between one molecule of the proteinaceous material and another molecule of the proteinaceous material, or between the proteinaceous material and the conductive element). Exemplary cross-linking methods include, but are not limited to, chemical reactions, irradiation, application of heat, dehydrothermal treatment, enzymatic treatment, and the like. In some embodiments, the cross-linking is via a cross-linking agent. Exemplary cross-linking agents are described herein. In some embodiments, the step of cross-linking the proteinaceous material is prior to the step of depositing the redox mediator and/or the oxidoreductase. In some other embodiments, the step of cross-linking the proteinaceous material is after the step of depositing the redox mediator and/or the oxidoreductase.

In some embodiments of any one of the aspects described herein, the method for preparing a surface with an antifouling coating layer comprises a step of adding a redox mediator to the antifouling coating layer. In some embodiments, the step of adding the redox mediator to the antifouling coating layer comprises conjugating, e.g., covalently linking the redox mediator to a component of the antifouling coating layer. For example, the step of adding the redox mediator to the antifouling coating layer comprises conjugating, e.g., covalently linking the redox mediator with the proteinaceous material and/or the conductive element in the antifouling coating layer. It is noted that the redox mediator can be present in the composition prior to the step of coating the surface.

If the proteinaceous material is cross-linked, the step of adding the redox mediator to the antifouling coating layer can be carried out prior to or after the cross-linking step. Accordingly, in some embodiments, the step of adding the redox mediator to the antifouling coating layer is prior to the step of cross-linking the proteinaceous material. In some other embodiments, the step of adding the redox mediator to the antifouling coating layer is after the step of cross-linking the proteinaceous material.

Embodiments of the various aspects described herein include denatured proteinaceous material. Accordingly, in some embodiments of the method for preparing a surface with an antifouling coating layer, the method comprises a step of denaturing the proteinaceous material. It is noted the proteinaceous material can be denatured prior to or after adding the redox mediator. Preferably, the proteinaceous material is denatured prior to adding the redox mediator.

In some embodiments of any one of the aspects described herein, the method for preparing a surface with an antifouling coating layer comprises a step of adding an oxidoreductase to the antifouling coating layer. In some embodiments, the step of adding the oxidoreductase to the antifouling coating layer comprises conjugating, e.g., covalently linking the oxidoreductase to a component of the antifouling coating layer. For example, the step of adding the oxidoreductase to the antifouling coating layer comprises conjugating, e.g., covalently linking the oxidoreductase with the proteinaceous material and/or the conductive element in the antifouling coating layer. It is noted that the oxidoreductase can be present in the composition prior to the step of coating the surface.

If the proteinaceous material is cross-linked, the step of adding the oxidoreductase to the antifouling coating layer can be carried out prior to or after the cross-linking step. Accordingly, in some embodiments, the step of adding the oxidoreductase to the antifouling coating layer is prior to the step of cross-linking the proteinaceous material. In some other embodiments, the step of adding the oxidoreductase to the antifouling coating layer is after the step of cross-linking the proteinaceous material.

Embodiments of the various aspects described herein include denatured proteinaceous material. Accordingly, in some embodiments of the method for preparing a surface with an antifouling coating layer, the method comprises a step of denaturing the proteinaceous material. It is noted the proteinaceous material can be denatured prior to or after adding the oxidoreductase. Preferably, the proteinaceous material is denatured prior to adding the oxidoreductase.

In some embodiments of any one of the aspects described herein, the method for preparing a surface with an antifouling coating layer further comprises a step of adding a target binding molecule to the antifouling coating layer. For example, the method comprises a step of coating a surface of the antifouling coating layer with a target binding molecule. In some embodiments, the step of adding the target binding molecule to the antifouling coating layer comprises conjugating, e.g., covalently linking the target binding molecule to a component of the antifouling coating layer. For example, the step of adding the target binding molecule to the antifouling coating layer comprises conjugating, e.g., covalently linking the target binding molecule with the proteinaceous material and/or the conductive element in the antifouling coating layer. It is noted that the target binding molecule can be present in the composition prior to the step of coating the surface.

If the proteinaceous material is cross-linked, the step of adding the target binding molecule to the antifouling coating layer can be carried out prior to or after the cross-linking step. Accordingly, in some embodiments, the step of adding the target binding molecule to the antifouling coating layer is prior to the step of cross-linking the proteinaceous material. In some other embodiments, the step of adding the target binding molecule to the antifouling coating layer is after the step of cross-linking the proteinaceous material.

Embodiments of the various aspects described herein include denatured proteinacaoue material. Accordingly, in some embodiments of the method for preparing a surface with an antifouling coating layer, the method comprises a step of denaturing the proteinaceous material. It is noted the proteinaceous material can be denatured prior to or after adding the target binding molecule. Preferably, the proteinaceous material is denatured prior to adding the target binding molecule.

The surface to be coated can be any surface. For example, the surface to be coated can be a surface of a conductive substrate, such as an electrically conductive substrate. In some embodiments, the antifouling coating layer is directly or indirectly connected with an electrode. For example, the surface to be coated is a surface, e.g., a conductive surface of an electrode. In some embodiments of any one of the aspects described herein, the surface to be coated is a surface of a medical device.

### Analyte Detection

The electrode described herein can be used for detecting analytes, e.g., in a sample. Accordingly, another aspect provided herein relates to methods of detecting at least one target analyte, including, e.g., at least 2, 3, 4, 5, 6, 7, 8 target analytes or more. Generally, the method comprises contacting a sample suspected of comprising a target analyte with an electrode or described herein and detecting oxidation or reduction of the target analyte by the oxidoreductase present in the antifouling coating layer on the electrode. The oxidation or reduction may be detected electrochemically. Optionally, detecting the oxidation or reduction comprises applying a voltage to the electrode and measuring the current generated from the electrode.

The applied voltage provides a sufficiently strong electric field to liberate H⁺ from H₂O₂, but not strong enough to cause electrolysis of water, as electrolysis of water may lead to a decrease in the signal-to-noise ratio (i.e. below −1.23 V). Typically, the voltage applied is between about 0 V and −2 V, e.g. about −1 V. In some embodiments, low voltages can be used including 250 to 500 mV to 300-400 mV. The voltage range can then be −0.25 to −2V or −0.25 to −0.1 V. Generally, the target analyte is a substrate for the oxidoreductase.

### Target Analytes

In some embodiments, the analyte is a biological analyte. In some embodiments, the analyte ion, molecule, oligomer, polymer, protein, peptide, polypeptide, peptidomimetic, nucleic acid, antigen, antibody, nucleic acid, toxin, biological threat agent such as spore, viral, cellular and protein toxin, carbohydrate, monosaccharide, disaccharide, oligosaccharide, polyol, and polysaccharide, lipid, peptidoglycan, cell, microbial matter, steroid, hormone, lipopolysaccharide, endotoxin, therapeutic agent, lipid-binding molecule, co-factor, small molecule, fatty acid, chemical, or combinations of these. The analyte is optionally an antigen or antibody indicative of infection or resistance to infection. The analyte is optionally a clinical chemistry analyte.

In some embodiments, the analyte is immunological or serological, for example an antigen or antibody.

In some embodiments the analyte is a hormone, for example a gynaecological hormone such as luteinizing hormone (LH), progesterone, estradiol or follicle-stimulating hormone. In preferred embodiments the probe detects LH. In some embodiments the probe is a LH specific antibody. In some embodiments the probe is an LH monoclonal antibody. Additionally, or alternatively the hormone may be a pregnancy hormone such as human chorionic gonatropin (hCG).

In some embodiments the analyte is a clinical chemistry analyte such as an ion, salt, mineral, metabolite, therapeutic drug, toxicology marker, drug of abuse, transport protein, enzyme, specific protein, lipoprotein or marker, for example diabetes or myocardial infarction markers. In some embodiments the analyte is a metabolite selected from the group of glucose, cholesterol, urea, lactic acid, bilirubin, creatinine, triglycerides. In preferred embodiments the probe is selected to detect glucose or cholesterol.

In some embodiments, the analyte is a tumour marker. Tumour markers can be used in guiding treatment decisions, monitoring treatment, predicting the change of recovery and to predict or monitor for tumour recurrence.

### Sample

In accordance with various embodiments described herein, a sample, including any fluid or specimen (processed or unprocessed) that is intended to be evaluated for the presence of an analyte can be subjected to methods, compositions, kits and systems described herein. The sample or fluid can be liquid, supercritical fluid, solutions, suspensions, gases, gels, slurries, and combinations thereof. The sample or fluid can be aqueous or non-aqueous.

In some embodiments, the sample can be an aqueous fluid. An aqueous fluid includes biological fluids as described below. Optionally, if the sample is water-based but not fluid, an aqueous solution can be added to produce a fluid sample.

In some embodiments, the sample can include a biological fluid obtained from a subject. Exemplary biological fluids obtained from a subject can include, but are not limited to, blood (including whole blood, plasma, cord blood and serum), lactation products (e.g., milk), amniotic fluids, sputum, saliva, urine, semen, cerebrospinal fluid, bronchial aspirate, perspiration, mucus, liquefied stool sample, synovial fluid, lymphatic fluid, tears, tracheal aspirate, and any combination thereof. In some embodiments, a biological fluid can include a homogenate of a tissue specimen (e.g., biopsy) from a subject. In one embodiment, a test sample can comprise a suspension obtained from homogenization of a solid sample or a fragment thereof obtained from a subject.

In some embodiments, the sample can include a fluid or specimen obtained from an environmental source. For example, the fluid or specimen obtained from the environmental source can be obtained or derived from food products or industrial food products, food produce, poultry, meat, fish, beverages, dairy products, water (including wastewater), surfaces, ponds, rivers, reservoirs, swimming pools, soils, food processing and/or packaging plants, agricultural places, hydrocultures (including hydroponic food farms), pharmaceutical manufacturing plants, animal colony facilities, and any combinations thereof.

In some embodiments, the sample can be a non-biological fluid. As used herein, the term “non-biological fluid” refers to any fluid that is not a biological fluid as the term is defined herein. Exemplary non-biological fluids include, but are not limited to, water, salt water, brine, drinking water, industrial water, brown water, sewerage, and mixtures thereof. Preferred non-biological fluids are drinking or industrial water or sewerage.

In some embodiments, the sample is pre-processed prior to contacting with the electrode or the sensor.

### Sensors

In another aspect provided herein is a sensor comprising an electrode as described herein or an as described herein.

In the context of this specification, the term “sensor” refers a device that senses the presence and/or amount of something. For example, the sensor could sense the presence of a chemical such as glucose, a protein such as an antigen, or an antibody in a biological fluid.

A sensor has two basic components: the sensing surface (or receptor) and the transducer. The sensing surface interacts with the target analyte and the transducer converts this interaction into a readable electronic signal. The sensor performance characteristics depend on both the components. The sensor selectivity and affinity towards the target analyte depends solely on the sensing surface because the analyte interacts only at the sensing surface. Other performance metrics such as sensitivity, resolution, and calibration depend on both components.

A sensor can be a carbon-based electrode. For example, a sensor can be a screen-printed electrode.

In some embodiments the sensor may be an eRapid chip. Such devices are generally described in “Enabling Multiplexed Electrochemical Detection of Biomarkers with High Sensitivity in Complex Biological Samples, Sanjay S. Timilsina, Pawan Jolly, Nolan Durr, Mohamed Yafia, and Donald E. Ingber, Acc. Chem. Res. 2021, 54, 18, 3529-3539”, which is hereby incorporated herein in its entirety.

The sensor may have a channel length of between about 5 μm and about 50 μm, or between about 10 μm and about 30 μm, or about 20 μm, and a channel width of between about 1 mm and about 20 mm, or between about 1 mm and about 10 mm, or about 3 mm.

In some embodiments, the sensor has one or more fluid-contact surfaces, and the electrode is immobilized on at least a portion of the fluid contact surface. In some embodiments, the sensor has one or more wells. In some embodiments, each well of the sensor comprises an inner bottom surface on which one or more analyte specific electrodes are immobilized. In some embodiments, the wells are open cells comprising open tops, enclosed sides and bottom, and one or more analyte-specific electrodes immobilized on the inner fluid-contact surface of the wells. In some embodiments, the sensor comprises 1, 2, 3, 4, 5, 6, 8, 10, 12, 16, 24, 32, 48, 64, 96 or more open wells. In one embodiment the sensor is in the form of a 96-well microtiter plate.

In some embodiments, the wells are microfluidic flow cells comprising an enclosed top, sides and bottom, wherein the top of each flow cell includes a fluid inlet and a fluid outlet, and comprising one or more analyte-specific electrodes immobilized on the inner fluid-contact surface of the wells. In some embodiments, the electrochemical sensor comprises 1, 2, 3, 4, 5, 6, 8, 10, 12, 16, 24, 32, 48, 64, 96 or more microfluidic flow cells. Another embodiment is in the form of a 96-well microtiter plate, wherein each well comprises an enclosed top having a fluid inlet and a fluid outlet. In some embodiments, the sensor comprises both one or more open cells and one or more flow cells. Each well contains an array of analyte-specific electrodes (e.g., 32 gold electrodes) that can be individually modified with capture probes to bind the corresponding target analyte (e.g., pathogen, protein, carbohydrate, toxin, drug, etc.) present in the collected sample. In some embodiments, one sample is introduced into each well. In embodiments having two or more wells, portions of the same sample can be introduced into more than one well, or different samples can be introduced into different wells. Thus, in embodiments having multiple wells, multiple samples can be simultaneously assayed.

Also provided herein is the use of a sensor. In some embodiments, the sensor may be for sensing an analyte in a sample. The analyte is optionally a biological analyte. In an embodiment the analyte is an antibody, antigen, protein, peptide or chemical. The sample may be any aqueous solution but is preferably a biological fluid, more preferably a bodily fluid, and still more preferably, saliva.

### Kits

In another aspect, the present disclosure provides a kit comprising a composition, surface, electrode, or sensor described herein.

In addition to the above-mentioned components, any embodiments of the kits described herein can include informational material. The informational material can be descriptive, instructional, marketing or other material that relates to the methods described herein and/or the use of the aggregates for the methods described herein. For example, the informational material can describe methods for using the kits provided herein to perform an assay for capture and/or detection of a target analyte. The kit can also include an empty container and/or a delivery device, e.g., which can be used to deliver a test sample to a test container.

The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is a link or contact information, e.g., a physical address, email address, hyperlink, website, or telephone number, where a user of the kit can obtain substantive information about the formulation and/or its use in the methods described herein. Of course, the informational material can also be provided in any combination of formats.

In some embodiments, the kit can contain separate containers, dividers or compartments for each component and informational material. For example, each different component can be contained in a bottle, vial, or syringe, and the informational material can be contained in a plastic sleeve or packet. In other embodiments, the separate elements of the kit are contained within a single, undivided container.

Some exemplary aspects of the disclosure are described by one or more of following numbered Embodiments:

Embodiment 1: An electrode comprising: (i) a conductive substrate (e.g., an electrically conductive substrate); and (ii) an antifouling coating layer on at least a portion of a surface of the conductive substrate, and wherein the antifouling coating layer comprises: a proteinaceous material, a conductive element, a redox mediator covalently linked to the proteinaceous material or the conductive element, and an oxidoreductase covalently linked to the proteinaceous material or the conductive element.

Embodiment 2: The electrode of embodiment 1, wherein the antifouling coating layer is adapted for contact with an analyte or a sample comprising an analyte.

Embodiment 3: The electrode of any one embodiments 1-2, wherein the antifouling coating layer is porous.

Embodiment 4: The electrode of any one of embodiments 1-3, wherein the antifouling coating layer has a porosity of about 5% to about 95%.

Embodiment 5: The electrode of any one of embodiments 1-4, wherein the antifouling coating layer comprises macropores (e.g., pores having a diameter of from about 0.1 μm to about 10 μm such as from about 0.5 μm to about 5 μm or from about 1 μm to about 3 μm).

Embodiment 6: The electrode of any one of embodiments 1-5, wherein the antifouling coating layer comprises mesopores (e.g., pores having a diameter from about 5 nm to about 99 nm).

Embodiment 7: The electrode of any one of embodiments 1-6, wherein the antifouling coating layer comprises nanopores (e.g., pores having a diameter of from about 0.1 nm to about 4.5 nm).

Embodiment 8: The electrode of any one of embodiments 1-7, wherein the redox mediator is covalently linked to the proteinaceous material or the conductive element by a cross-linker.

Embodiment 9: The electrode of embodiment 8, wherein the cross-linker is glutaraldehyde, genipin, polyethylene glycol, or a carbodiimide based cross-linker.

Embodiment 10: The electrode of any one of embodiments 1-9, wherein the redox mediator is covalently linked with the proteinaceous material.

Embodiment 11: The electrode of any one of embodiments 1-10, wherein the redox mediator is covalently linked with the conductive element.

Embodiment 12: The electrode of any one of embodiments 1-11, wherein the redox mediator is on a surface of the antifouling coating layer.

Embodiment 13: The electrode of any one of embodiments 1-12, wherein the redox mediator is embedded within the antifouling coating layer.

Embodiment 14: The electrode of any one of embodiments 1-13, wherein the redox mediator is selected from the group consisting of metallocenes metallocence derivatives, quinones, polycyclic aromatic hydrocarbons, redox-active organic molecules, phenazines, organosulfur compounds, and radical acceptors.

Embodiment 15: The electrode of any one of embodiments 1-14, wherein the redox mediator is selected from the group consisting of ferrocene, ferrocene derivatives, 3,3′,5,5′-tetramethylbenzidine (TMB), o-phenylenediamine dihydrochloride (OPD), N,N,N′,N′-tetramethyl-p-phenylenediamine, viologens 2,2′-azino-bis(3-ethylbenzothiazoline-6-sulfonic acid) (ABTS), p-nitrophenyl Phosphate (PNPP), 3,3′-diaminobenzidine (DAB), 4-chloro-1-naphthol (4-CN), 5-bromo-4-chloro-3-indolyl-phosphate (BCIP), nitro blue tetrazolium (NBT), methylene blue, tetrathialfulvaene, 2,6-dichloroindophenol (DCIP), 2,6-dichloroindophenyl phosphate, riboflavin 5′-monophosphate (RMP), ethyl viologen (1,1′-bis(ethyl)-4,4′-bipyridinium or BEBP), Paraquat, hexaamine ruthenium, 1,4-naphthoquione, resorufine, cyanomethylviologen, diquat, triquat, 1,1′-bis(2-sulfoethyl)-4,4′-bipyridinium, 1,1′-dibenzyl-4,4′-bipyridinium, 4,4′-dicarboxy-2,2′-bipyridyl, 1-hydroxybenzotriazole, veratryl alcohol, violuric acid, 2-methoxy-phenothiazone, 3-hydroxyanthranilic acid, anthraquinone 2,6-disulfonic acid, N-hydroxyacetanilide, phenol red, 3,3′,5,5′-tetramethyl benzidine, dichlorophenol red, 2,2′,6,6′-tetramethylpiperidine-N-oxyl radical, syringaldehyde and acetosyringone, 2,2,6,6-tetramethylpiperidin-1-yl)oxyl (TEMPO), 2,2′7,7′-tetrakis-(N,N-di-p-methoxyphenyl-amine)-9,9′-spirobifluorene (spiro-MeOTAD), sodium anthraquinone-2,6-di sulphonate (AQDS), benzoquinones, 2,2′-biimidazole, 2-(2-pyridyl) imidazole, 2,2′-bipyridine, tetracyanoquinodimethane (TCNQ), and combinations thereof.

Embodiment 16: The electrode of any one of embodiments 1-15, wherein the redox mediator is ferrocene or a derivative thereof.

Embodiment 17: The electrode of any one of embodiments 1-16, wherein the redox mediator is selected from the group consisting of aminoferrocene, ferrocene monocarboxylic acid, ferrocyanide, 1,1′-ferrocene dicarboxylic acid, 1,1′-dimethylferrocene (DMF), polyvinylferrocene, [N-ferrocenoyl]-4-aminophenyl phosphate, ferrocenylmethyl methacrylate, β-ferrocenyl-propenoic acid, and ferrocene monocarboxylic acid (FMCA).

Embodiment 18: The electrode of any one of embodiments 1-17, wherein the redox mediator is aminoferrocene.

Embodiment 19: The electrode of any one of embodiments 1-18, wherein the redox mediator is present in an amount from about 0.001 to about 10 (w/w).

Embodiment 20: The electrode of any one of embodiments 1-19, wherein a ratio of the redox mediator to the oxidoreductase is from about 1:100 to about 1:1 (w/w).

Embodiment 21: The electrode of any one of embodiments 1-20, wherein a ratio of the redox mediator to the proteinaceous material is from about 1:10 to about 1:1000 (w/w).

Embodiment 22: The electrode of any one of embodiments 1-21, wherein a ratio of the redox mediator to the conductive element is from about 1:10 to about 1:1000 (w/w).

Embodiment 23: The electrode of any one of embodiments 1-22, wherein the oxidoreductase is covalently linked to the proteinaceous material or the conductive element by a cross-linker.

Embodiment 24: The electrode of embodiment 23, wherein the cross-linker is glutaraldehyde, genipin, or polyethylene glycol.

Embodiment 25: The electrode of any one of embodiments 1-24, wherein the oxidoreductase is covalently linked with the proteinaceous material.

Embodiment 26: The electrode of any one of embodiments 1-25, wherein the oxidoreductase is covalently linked with the conductive element.

Embodiment 27: The electrode of any one of embodiments 1-26, wherein the oxidoreductase is on a surface of the antifouling coating layer.

Embodiment 28: The electrode of any one of embodiments 1-27, wherein the oxidoreductase is embedded within the antifouling coating layer.

Embodiment 29: The electrode of any one of embodiments 1-28, wherein the oxidoreductase is selected from the group consisting of oxidases, reductases, dehydrogenases, peroxidases, oxygenases, monooxygenases, dioxygenases, lipoxygenases, hydrogenases, transhydrogenases, peroxidases, catalases, epoxidases, hydroxylases, demethylases, desaturases, dismutases, hydroxyltransferases, synthases, dehalogenases, and deiodinases.

Embodiment 30: The electrode of any one of embodiments 1-29, wherein the oxidoreductase is selected from the group consisting of glucose oxidase, malate oxidase, hexose oxidase, aryl-alcohol oxidase, alcohol oxidase, long-chain alcohol oxidase, glycerol-3-phosphate oxidase, poly vinyl-alcohol oxidase, D-arabinono-1,4-lactone oxidase, D-mannitol oxidase, xylitol oxidase, oxalate oxidase, carbon-monoxide oxidase, 4-hydroxyphenylpyruvate oxidase, dihydrouracil oxidase, ethanolamine oxidase, lactate oxidase, L-aspartate oxidase, sarcosine oxidase, urate oxidase, methanethiol oxidase, 3-hydroxyanthranilate oxidase, cholesterol oxidase, xanthine oxidase, amino-acid oxidase, laccase, catalase, fatty-acid peroxidase, peroxidase, diarylpropane peroxidase, ferroxidase, pteridine oxidase, columbamine oxidase, catechol 1,2-dioxygenase, gentisate 1,2-dioxygenase, homogentisate 1,2-dioxygenase, lipoxygenase, ascorbate 2,3-dioxygenase, 3-carboxyethylcatechol 2,3-dioxygenase, indole 2,3-dioxygenase, caffeate 3,4-dioxygenase, arachidonate 5-lipoxygenase, biphenyl-2,3-diol 1,2-dioxygenase, linoleate 11-lipoxygenase, acetylacetone-cleaving enzyme, lactate 2-monooxygenase, phenylalanine 2-monooxygenase, inositol oxygenase, fructose dehydrogenase, alcohol dehydrogenase, glycerol dehydrogenase, propanediol-phosphate dehydrogenase, L-lactate dehydrogenase, D-lactate dehydrogenase, glycerate dehydrogenase, glucose 1-dehydrogenase, galactose 1-dehydrogenase, allyl-alcohol dehydrogenase, 4-hydroxybutyrate dehydrogenase, octanol dehydrogenase, aryl-alcohol dehydrogenase, cyclopentanol dehydrogenase, long-chain-3-hydroxyacyl-CoA dehydrogenase, butanal dehydrogenase, terephthalate 1,2-cis-dihydrodiol dehydrogenase, succinate dehydrogenase, glutamate dehydrogenase, glycine dehydrogenase, hydrogen dehydrogenase, 4-cresol dehydrogenase, phosphonate dehydrogenase, diethyl 2-methyl-3-oxosuccinate reductase, tropinone reductase, long-chain-fatty-acyl-CoA reductase, carboxylate reductase, D-proline reductase, glycine reductlactase, Heme-proteins such as cytochromes, carbon-carbon lyases, carbon-oxygen lyases, carbon-nitrogen lyases, carbon-sulfur lyases, carbon-halide lyases, and phosphorus-oxygen lyases, alkaline phosphatase, malate dehydrogenase, staphylococcal nuclease, delta-V-steroid isomerase, yeast alcohol dehydrogenase, alpha-glycerophosphate dehydrogenase, triose phosphate isomerase, horseradish peroxidase (HRP), asparaginase, beta-galactosidase, ribonuclease, urease, catalase, glucose-VI-phosphate dehydrogenase, glucoamylase, tyrosinase, pyruvate oxidase, aldehyde oxidase, carbon-monoxide oxidase, retinal oxidase, abscisic-aldehyde oxidase, (methyl) glyoxal oxidase, xanthine oxidase, oxalate oxidase, and acetylcholinesterase.

Embodiment 31: The electrode of any one of embodiments 1-30, wherein the oxidoreductase is glucose oxidase.

Embodiment 32: The electrode of any one of embodiments 1-31, wherein the oxidoreductase is present in an amount from about 20% to about 80% (w/w).

Embodiment 33: The electrode of any one of embodiments 1-32, wherein a ratio of the oxidoreductase to the proteinaceous material is about 10:1 to about 5:1 (w/w).

Embodiment 34: The electrode of any one of embodiments 1-33, wherein a ratio of the oxidoreductase to the conductive element is about 10:1 to about 5:1 (w/w).

Embodiment 35: The electrode of any one of embodiments 1-34, wherein the conducting material comprises conductive particles, conductive rods, conductive fibers, conductive nano-particles, conductive polymers, conductive nano-flakes, conductive nanotubes, semi-conductive particles, semi-conductive rods, semi-conductive fibers, semi-conductive nano-particles, semi-conductive nano-flakes, semi-conductive nanotubes, or semi-conductive polymers.

Embodiment 36: The electrode of any one of embodiments 1-35, wherein the conducting material is a metal, a metalloid, conducting polymer, a conducting carbon-based material, organic compounds having conducting and/or semiconducting properties, or any combination thereof.

Embodiment 37: The electrode of any one of embodiments 1-36, wherein the conducting material comprises an allotrope of carbon atoms arranged in a hexagonal lattice.

Embodiment 38: The electrode of any one of embodiments 1-37, wherein the conducting material is graphite, functionalised graphene oxide, reduced graphene oxide, or carbon nano-tubes (CNTs).

Embodiment 39: The electrode of embodiment 38, wherein the carbon nanotubes are carboxylated carbon nanotubes or aminated carbon nanotubes.

Embodiment 40: The electrode of embodiment 38, wherein the reduced graphene oxide is a carboxylated reduced graphene oxide or an aminated reduced graphene oxide.

Embodiment 41: The electrode of any one of embodiments 1-36, wherein the conductive material comprises gold.

Embodiment 42: The electrode of any one of embodiments 1-36, wherein the conductive material comprises one or more organic compounds having conducting and/or semiconducting properties.

Embodiment 43: The electrode of embodiment 42, wherein the organic compounds having conducting and/or semiconducting properties are selected from the group consisting of polyacetylenes, porphyrins, phthalocyanins, fullerenes, polyparaphenylenes, polyphenylenevinylenes, polyfluorenes, polythiophenes, polypyrroles, polypyridines, polycarbazoles, polypyridinevinylenes, polyarylvinylenes, poly(p-phenylmethylvinylenes), derivatives and co-polymers thereof, and any combination thereof.

Embodiment 44: The electrode of embodiment 42 or 43, wherein the organic compounds having conducting and/or semiconducting properties are selected from the group consisting of poly(3-hexylthiophene) (P3HT), perfluoro-3,6-dioxa-4-methyl-7-octenesulfonic acid copolymer, tetrafluoroethylene-perfluoro-3,6-dioxa-4-methyl-7-octenesulfonic acid copolymer (e.g. nafion), poly(9,9-dioctylfluorene-2,7-diyl-co-bis-N,N-(4-butylphenyl)-bis-N,N-phenyl-1,4-phenylenediamine), poly(9,9-dioctylfluorene-2,7-diyl-co-benzothiadiazole), (6,6)-phenyl-C61-butyric acid methyl ester, poly(2-methoxy-5-(2′-ethyl-hexyloxy)-1,4-phenylene vinylene), poly(4-vinylphenol) (PVP), and copolymers thereof, and any combination thereof.

Embodiment 45: The electrode of any one of embodiments 1-44, wherein the conductive element is present in an amount from about 0.01% to about 10% (w/v).

Embodiment 46: The electrode of any one of embodiments 1-45, wherein a ratio of the proteinaceous material to the conductive element is from about 10:1 to about 1:1 (w/w).

Embodiment 47: The electrode of any one of embodiments 1-46, wherein the proteinaceous material is denatured.

Embodiment 48: The electrode of any one of embodiments 1-47, wherein the proteinaceous material is non-reversibly denatured.

Embodiment 49: The electrode of any one of embodiments 1-48, wherein the proteinaceous material is a globular protein.

Embodiment 50: The electrode of any one of embodiments 1-49, wherein the proteinaceous material is a non-glycosylated protein.

Embodiment 51: The electrode of any one of embodiments 1-50, wherein the proteinaceous material is a serum albumin protein.

Embodiment 52: The electrode of any one of embodiments 1-51, wherein the proteinaceous material is bovine serum albumin (BSA) or human serum albumin (HSA).

Embodiment 53: The electrode of any one of embodiments 1-52, wherein the proteinaceous material is cross-linked with the conductive element.

Embodiment 54: The electrode of any one of embodiments 1-53, wherein the proteinaceous material is cross-linked to itself.

Embodiment 55: The electrode of any one of embodiments 1-54, wherein the proteinaceous material is covalently linked to the conductive surface.

Embodiment 56: The electrode of any one of embodiments 1-55, wherein the proteinaceous material is cross-linked by a cross-linker.

Embodiment 57: The electrode of embodiment 53, wherein the cross-linker is glutaraldehyde, genipin, or polyethylene glycol.

Embodiment 58: The electrode of any one of embodiments 1-57, wherein the antifouling coating layer further comprises a target binding molecule capable of binding with a target molecule.

Embodiment 59: The electrode of embodiment 58, wherein the target binding molecule is covalently linked to the proteinaceous material, or the conductive or semi-conductive material.

Embodiment 60: The electrode of embodiment 58 or 59, wherein the target binding molecule is a receptor, a ligand for a receptor, an antibody, antigen binding fragment of an antibody, an antigen, an enzyme or a nucleic acid.

Embodiment 61: The electrode of any one of embodiments 58-60, wherein the target binding molecule is an antibody, an antigen binding fragment of an antibody or an antigen.

Embodiment 62: The electrode of any one of the preceding embodiments, wherein the antifouling coating layer comprises an anti-microbial agent.

Embodiment 63: The electrode of embodiment 63, wherein the anti-microbial agent is an anti-bacterial agent, anti-fungal agent or anti-viral agent.

Embodiment 64: The electrode of embodiment 62 or 63, wherein the anti-microbial agent is an anti-bacterial agent.

Embodiment 65: The electrode of embodiment 64, wherein the anti-bacterial agent is selected from the group consisting of macrolides or ketolides such as erythromycin, azithromycin, clarithromycin, and telithromycin; beta-lactams including penicillin, cephalosporin, and carbapenems such as carbapenem, imipenem, and meropenem; monolactams such as penicillin G, penicillin V, methicillin, oxacillin, cloxacillin, dicloxacillin, nafcillin, ampicillin, amoxicillin, carbenicillin, ticarcillin, meziocillin, piperacillin, azlocillin, temocillin, cepalothin, cephapirin, cephradine, cephaloridine, cefazolin, cefamandole, cefuroxime, cephalexin, cefprozil, cefaclor, loracarbef, cefoxitin, cefmetazole, cefotaxime, ceftizoxime, cefiriaxone, cefoperazone, ceftazidime, cefixime, cefpodoxime, ceftibuten, cefdinir, cefpirome, cefepime, cefadroxil, ceftriaxone, ceftobiprole and astreonam; quinolones such as nalidixic acid, oxolinic acid, norfloxacin, pefloxacin, enoxacin, ofloxacin, levofloxacin, ciprofloxacin, temafloxacin, lomefloxacin, fleroxacin, grepafloxacin, sparfloxacin, trovafloxacin, clinafloxacin, gatifloxacin, moxifloxacin, sitafloxacin, ganefloxacin, gemifloxacin and pazufloxacin; antibacterial sulfonamides and antibacterial sulphanilamides, including para-aminobenzoic acid, sulfadiazine, sulfisoxazole, sulfamethoxazole and sulfathalidine; aminoglycosides such as streptomycin, neomvcin, kanamycin, paromycin, gentamicin, tobramycin, amikacin, netilmicin, spectinomycin, sisomicin, dibekalin and isepamicin; tetracyclines such as tetracycline, chlortetracycline, demeclocycline, minocycline, oxytetracycline, methacycline, doxycycline; rifamycins such as rifampicin (also called rifampin), rifapentine, rifabutin, bezoxazinorifamycin and rifaximin; lincosamides such as lincomycin and clindamycin; glycopeptides such as vancomycin and teicoplanin; streptogramins such as quinupristin and daflopristin; oxazolidinones such as linezolid; polymyxin, colistin and colymycin; trimethoprim, bacitracin, and phosphonomycin.

Embodiment 66: The electrode of embodiment 62 or 63, wherein the anti-microbial agent is an antifungal agent.

Embodiment 67: The electrode of embodiment 66, wherein the antifungal agent is selected from the group consisting of azoles (e.g., barleyconazole, butoconazole, clortrimazole, econazole, fluconazole, isavuconazole, itraconazole, ketoconazole, miconazole, oxyconazole, posaconazole, ravuconazole, saperconazole, sulconazole, tercocnazole, tioconazole, voriconazole, and ciclopirox), polyenes (e.g., natamycin, lucensomycin, nystatin, amphotericin B, etc.), echinocandins (e.g., Cancidas), pradimicins (e.g., beanomicins, nikkomycins, sordarins, allylamines, etc.), Triclosan, Piroctone, fenpropimorph, terbinafine, cyclopyroxolamine, flucitocin, griseofulvin haloprozin, tolnaftate, naphthypine, hydrochloride, morpholine, butenapin, undecylenic acid, propionic acid, and derivatives and analogs thereof.

Embodiment 68: The electrode of embodiment 62 or 63, wherein the anti-microbial agent is an antimicrobial peptide or polymer.

Embodiment 69: The electrode of embodiment 62 or 63, wherein the anti-microbial agent is a metal particle.

Embodiment 70: The electrode of embodiment 69, wherein anti-microbial agent is titanium oxide, copper, or silver nanoparticles.

Embodiment 71: The electrode of any one of embodiments 1-70, wherein the antifouling coating layer further comprises a therapeutic agent, e.g., anti-inflammatory drugs sirolimus, everolimus, biolimus (A9), zotarolimus (ABT-578), tacrolimus, and pimecrolimus, genistein, steroids (dexamethasone, prednisolone, methylprednisolone and hydrocortisone), fluocinolone acetonide, hormones etc.

Embodiment 72: The electrode of any one of embodiments 1-71, wherein the antifouling coating layer further comprises a polymer.

Embodiment 73: The electrode of embodiment 72, wherein the polymer is a water miscible polymer.

Embodiment 74: The electrode of embodiment 72 or 73, wherein the polymer is a degradable polymer

Embodiment 75: The electrode of any one of embodiments 72-74, wherein the polymer is selected from the group consisting of poly(N-isopropyl acrylamide) (PNIPAAm), polyethylene glycol, alginate, polytetrafluoroethylene (PTFE), polyacetylene (PA), polyaniline (PANI), polypyrrole (PPy), polythiophene (PTH), poly(para-phenylene) (PPP), poly(phenylenevinylene) (PPV), and polyfuran (PF).

Embodiment 76: The electrode of any one of embodiments 1-75, wherein the electrode is a planar or 3-dimensional electrode.

Embodiment 77: The electrode of any one of embodiments 1-76, wherein the conductive substrate comprises gold, silver, copper, platinum, aluminum, stainless steel, tungsten, indium tin oxide, titanium, lead, nickel, palladium, zirconium, niobium, tantalum, chromium, molybdenum, manganese, rhenium, ruthenium, rhodium, iridium, silicon, polyimide, parylene, benzocyclobutene, carbon, graphite, or any combination thereof.

Embodiment 78: The electrode of any one of embodiments 1-77, wherein the conductive substrate comprises a flexible substrate.

Embodiment 79: The electrode of embodiment 78, wherein the flexible substrate comprises polyethylene terephthalate, polyethylene naphathalate, polyimides, polymeric hydrocarbons, celluloses, plastics, polycarbonates, polystyrenes, or any combination thereof.

Embodiment 80: The electrode of any one of embodiments 1-79, wherein the coating layer has a thickness from about 2 nm to about 100 μm.

Embodiment 81: A sensor comprising an electrode of any one embodiments 1-80.

Embodiment 82: The sensor of embodiment 81, wherein the sensor comprises a fluid-contact surface and the electrode is immobilized on at least a portion of the fluid-contact surface.

Embodiment 83: The sensor of any one of embodiments 81-82, wherein the sensor comprises one or more microfluidic flow cells.

Embodiment 84: The sensor of any one of embodiments 81-83, wherein the fluid-contact surface further comprises a positive control electrode and/or a negative control electrode immobilized thereon.

Embodiment 85: A composition comprising: a proteinaceous material, a conductive element, a redox mediator covalently linked to the proteinaceous material or the conductive element, and an oxidoreductase covalently linked to the proteinaceous material or the conductive element.

Embodiment 86: The composition of embodiment 85, wherein the redox mediator is covalently linked to the proteinaceous material or the conductive element by a cross-linker.

Embodiment 87: The composition of embodiment 86, wherein the cross-linker is glutaraldehyde, genipin, or polyethylene glycol.

Embodiment 88: The composition of any one of embodiments 85-87, wherein the redox mediator is covalently linked with the proteinaceous material.

Embodiment 89: The composition of any one of embodiments 85-88, wherein the redox mediator is covalently linked with the conductive element.

Embodiment 90: The composition of any one of embodiments 85-89, wherein the redox mediator is selected from the group consisting of metallocenes metallocence derivatives, quinones, polycyclic aromatic hydrocarbons, redox-active organic molecules, phenazines, organosulfur compounds, and radical.

Embodiment 91: The composition of any one of embodiments 85-90, wherein the redox mediator is selected from the group consisting of ferrocene, ferrocene derivatives, 3,3′,5,5′-tetramethylbenzidine (TMB), o-phenylenediamine dihydrochloride (OPD), N,N,N′,N′-tetramethyl-p-phenylenediamine, viologens 2,2′-azino-bis(3-ethylbenzothiazoline-6-sulfonic acid) (ABTS), p-nitrophenyl Phosphate (PNPP), 3,3′-diaminobenzidine (DAB), 4-chloro-1-naphthol (4-CN), 5-bromo-4-chloro-3-indolyl-phosphate (BCIP), nitro blue tetrazolium (NBT), methylene blue, tetrathialfulvaene, 2,6-dichloroindophenol (DCIP), 2,6-dichloroindophenyl phosphate, riboflavin 5′-monophosphate (RMP), ethyl viologen (1,1′-bis(ethyl)-4,4′-bipyridinium or BEBP), Paraquat, hexaamine ruthenium, 1,4-naphthoquione, resorufine, cyanomethylviologen, diquat, triquat, 1,1′-bis(2-sulfoethyl)-4,4′-bipyridinium, 1,1′-dibenzyl-4,4′-bipyridinium, 4,4′-dicarboxy-2,2′-bipyridyl, 1-hydroxybenzotriazole, veratryl alcohol, violuric acid, 2-methoxy-phenothiazone, 3-hydroxyanthranilic acid, anthraquinone 2,6-disulfonic acid, N-hydroxyacetanilide, phenol red, 3,3′,5,5′-tetramethyl benzidine, dichlorophenol red, 2,2′,6,6′-tetramethylpiperidine-N-oxyl radical, syringaldehyde and acetosyringone, 2,2,6,6-tetramethylpiperidin-1-yl)oxyl (TEMPO), 2,2′7,7′-tetrakis-(N,N-di-p-methoxyphenyl-amine)-9,9′-spirobifluorene (spiro-MeOTAD), sodium anthraquinone-2,6-di sulphonate (AQDS), benzoquinones, 2,2′-biimidazole, 2-(2-pyridyl) imidazole, 2,2′-bipyridine, tetracyanoquinodimethane (TCNQ), and combinations thereof.

Embodiment 92: The composition of any one of embodiments 85-91, wherein the redox mediator is ferrocene or a derivative thereof.

Embodiment 93: The composition of any one of embodiments 85-92, wherein the redox mediator is selected from the group consisting of aminoferrocene, ferrocene monocarboxylic acid, ferrocyanide, 1,1′-ferrocene dicarboxylic acid, 1,1′-dimethylferrocene (DMF), polyvinylferrocene, [N-ferrocenoyl]-4-aminophenyl phosphate, ferrocenylmethyl methacrylate, and ferrocene monocarboxylic acid (FMCA).

Embodiment 94: The composition of any one of embodiments 85-93, wherein the redox mediator is aminoferrocene.

Embodiment 95: The composition of any one of embodiments 85-94, wherein the redox mediator is present in an amount from about 0.001 to about 10 (w/w).

Embodiment 96: The composition of any one of embodiments 85-95, wherein a ratio of the redox mediator to the oxidoreductase is from about 1:100 to about 1:1 (w/w).

Embodiment 97: The composition of any one of embodiments 85-96, wherein a ratio of the redox mediator to the proteinaceous material is from about 1:10 to about 1:1000 (w/w). The composition of any one of the preceding embodiments, wherein a ratio of the redox mediator to the conductive element is from about 1:10 to about 1:1000 (w/w).

Embodiment 98: The composition of any one of embodiments 85-97, wherein the oxidoreductase is covalently linked to the proteinaceous material or the conductive element by a cross-linker.

Embodiment 99: The composition of embodiment 98, wherein the cross-linker is glutaraldehyde, genipin, or polyethylene glycol.

Embodiment 100: The composition of any one of embodiments 85-99, wherein the oxidoreductase is covalently linked with the proteinaceous material.

Embodiment 101: The composition of any one of embodiments 85-100, wherein the oxidoreductase is covalently linked with the conductive element.

Embodiment 102: The composition of any one of embodiments 85-101, wherein the oxidoreductase is selected from the group consisting of oxidases, reductases, dehydrogenases, peroxidases, oxygenases, monooxygenases, dioxygenases, lipoxygenases, hydrogenases, transhydrogenases, peroxidases, catalases, epoxidases, hydroxylases, demethylases, desaturases, dismutases, hydroxyltransferases, synthases, dehalogenases, and deiodinases.

Embodiment 103: The composition of any one of embodiments 85-102, wherein the oxidoreductase is selected from the group consisting of glucose oxidase, malate oxidase, hexose oxidase, aryl-alcohol oxidase, alcohol oxidase, long-chain alcohol oxidase, glycerol-3-phosphate oxidase, poly vinyl-alcohol oxidase, D-arabinono-1,4-lactone oxidase, D-mannitol oxidase, xylitol oxidase, oxalate oxidase, carbon-monoxide oxidase, 4-hydroxyphenylpyruvate oxidase, dihydrouracil oxidase, ethanolamine oxidase, lactate oxidase, L-aspartate oxidase, sarcosine oxidase, urate oxidase, methanethiol oxidase, 3-hydroxyanthranilate oxidase, cholesterol oxidase, xanthine oxidase, amino-acid oxidase, laccase, catalase, fatty-acid peroxidase, peroxidase, diarylpropane peroxidase, ferroxidase, pteridine oxidase, columbamine oxidase, catechol 1,2 -dioxygenase, gentisate 1,2-dioxygenase, homogentisate 1,2-dioxygenase, lipoxygenase, ascorbate 2,3-dioxygenase, 3-carboxyethylcatechol 2,3-dioxygenase, indole 2,3-dioxygenase, caffeate 3,4-dioxygenase, arachidonate 5-lipoxygenase, biphenyl-2,3-diol 1,2-dioxygenase, linoleate 11-lipoxygenase, acetylacetone-cleaving enzyme, lactate 2-monooxygenase, phenylalanine 2-monooxygenase, inositol oxygenase, fructose dehydrogenase, alcohol dehydrogenase, glycerol dehydrogenase, propanediol-phosphate dehydrogenase, L-lactate dehydrogenase, D-lactate dehydrogenase, glycerate dehydrogenase, glucose 1-dehydrogenase, galactose 1-dehydrogenase, allyl-alcohol dehydrogenase, 4-hydroxybutyrate dehydrogenase, octanol dehydrogenase, aryl-alcohol dehydrogenase, cyclopentanol dehydrogenase, long-chain-3-hydroxyacyl-CoA dehydrogenase, butanal dehydrogenase, terephthalate 1,2-cis-dihydrodiol dehydrogenase, succinate dehydrogenase, glutamate dehydrogenase, glycine dehydrogenase, hydrogen dehydrogenase, 4-cresol dehydrogenase, phosphonate dehydrogenase, diethyl 2-methyl-3-oxosuccinate reductase, tropinone reductase, long-chain-fatty-acyl-CoA reductase, carboxylate reductase, D-proline reductase, glycine reductlactase, Heme-proteins such as cytochromes, carbon-carbon lyases, carbon-oxygen lyases, carbon-nitrogen lyases, carbon-sulfur lyases, carbon-halide lyases, and phosphorus-oxygen lyases, alkaline phosphatase, malate dehydrogenase, staphylococcal nuclease, delta-V-steroid isomerase, yeast alcohol dehydrogenase, alpha-glycerophosphate dehydrogenase, triose phosphate isomerase, horseradish peroxidase (HRP), asparaginase, beta-galactosidase, ribonuclease, urease, catalase, glucose-VI-phosphate dehydrogenase, glucoamylase, tyrosinase, pyruvate oxidase, aldehyde oxidase, carbon-monoxide oxidase, retinal oxidase, abscisic-aldehyde oxidase, (methyl) glyoxal oxidase, xanthine oxidase, oxalate oxidase, and acetylcholinesterase.

Embodiment 104: The composition of any one of embodiments 85-103, wherein the oxidoreductase is glucose oxidase.

Embodiment 105: The composition of any one of embodiments 85-104, wherein the oxidoreductase is present in an amount from about 20% to about 80% (w/w).

Embodiment 106: The composition of any one of embodiments 85-105, wherein a ratio of the oxidoreductase to the proteinaceous material is about 10:1 to about 5:1 (w/w).

Embodiment 107: The composition of any one of embodiments 85-106, wherein a ratio of the oxidoreductase to the conductive element is about 10:1 to about 5:1 (w/w).

Embodiment 108: The composition of any one of embodiments 85-107, wherein the conducting material comprises conductive particles, conductive rods, conductive fibers, conductive nano-particles, conductive polymers, conductive nano-flakes, conductive nanotubes, semi-conductive particles, semi-conductive rods, semi-conductive fibers, semi-conductive nano-particles, semi-conductive nano-flakes, semi-conductive nanotubes, or semi-conductive polymers.

Embodiment 109: The composition of any one of embodiments 85-108, wherein the conducting material is a metal, a metalloid, conducting polymer, a conducting carbon based material, organic compounds having conducting and/or semiconducting properties, or any combination thereof.

Embodiment 110: The composition of any one of embodiments 85-109, wherein the conducting material comprises an allotrope of carbon atoms arranged in a hexagonal lattice.

Embodiment 111: The composition of any one of embodiments 85-110, wherein the conducting material is graphite, functionalised graphene oxide, reduced graphene oxide, or carbon nano-tubes (CNTs)

Embodiment 112: The composition of embodiment 111, wherein the carbon nanotubes are carboxylated carbon nanotubes or aminated carbon nanotubes.

Embodiment 113: The composition of embodiment 112, wherein the reduced graphene oxide is a carboxylated reduced graphene oxide or an aminated reduced graphene oxide.

Embodiment 114: The composition of any one of embodiments 85-109, wherein the conductive material comprises gold.

Embodiment 115: The composition of any one of embodiments 85-109, wherein the conductive material comprises one or more organic compounds having conducting and/or semiconducting properties.

Embodiment 116: The composition of embodiment 115, wherein the organic compounds having conducting and/or semiconducting properties are selected from the group consisting of polyacetylenes, porphyrins, phthalocyanins, fullerenes, polyparaphenylenes, polyphenylenevinylenes, polyfluorenes, polythiophenes, polypyrroles, polypyridines, polycarbazoles, polypyridinevinylenes, polyarylvinylenes, poly(p-phenylmethylvinylenes), derivatives and co-polymers thereof, and any combination thereof.

Embodiment 117: The composition of embodiment 115 or 116, wherein the organic compounds having conducting and/or semiconducting properties are selected from the group consisting of poly(3-hexylthiophene) (P3HT), perfluoro-3,6-dioxa-4-methyl-7-octenesulfonic acid copolymer, tetrafluoroethylene-perfluoro-3,6-dioxa-4-methyl-7-octenesulfonic acid copolymer (e.g. nafion), poly(9,9-dioctylfluorene-2,7-diyl-co-bis-N,N-(4-butylphenyl)-bis-N,N-phenyl-1,4-phenylenediamine), poly(9,9-dioctylfluorene-2,7-diyl-co-benzothiadiazole), (6,6)-phenyl-C61-butyric acid methyl ester, poly(2-methoxy-5-(2′-ethyl-hexyloxy)-1,4-phenylene vinylene), poly(4-vinylphenol) (PVP), and copolymers thereof, and any combination thereof.

Embodiment 118: The composition of any one of embodiments 85-117, wherein the conductive element is present in an amount from about 0.01% to about 10% (w/w).

Embodiment 119: The composition of any one of embodiments 85-118, wherein a ratio of the proteinaceous material to the conductive element is from about 10:1 to about 1:1 (w/w).

Embodiment 120: The composition of any one of embodiments 85-119, wherein the proteinaceous material is denatured.

Embodiment 121: The composition of any one of embodiments 85-120, wherein the proteinaceous material is non-reversibly denatured.

Embodiment 122: The composition of any one of embodiments 85-121, wherein the proteinaceous material is a globular protein.

Embodiment 123: The composition of any one of embodiments 85-122, wherein the proteinaceous material is a non-glycosylated protein.

Embodiment 124: The composition of any one of embodiments 85-123, wherein the proteinaceous material is a serum albumin protein.

Embodiment 125: The composition of any one of embodiments 85-124, wherein the proteinaceous material is bovine serum albumin (BSA) or human serum albumin (HSA).

Embodiment 126: The composition of any one of embodiments 85-125, wherein the proteinaceous material is cross-linked with the conductive element.

Embodiment 127: The composition of any one of embodiments 85-126, wherein the proteinaceous material is cross-linked to itself.

Embodiment 128: The composition of any one of embodiments 85-127, wherein the proteinaceous material is cross-linked by a cross-linker.

Embodiment 129: The composition of embodiment 128, wherein the cross-linker is glutaraldehyde, genipin, or polyethylene glycol.

Embodiment 130: The composition of any one of embodiments 85-129, wherein the composition further comprises a target binding molecule capable of binding with a target molecule.

Embodiment 131: The electrode of embodiment 130, wherein the target binding molecule is covalently linked to the proteinaceous material, or the conductive or semi-conductive material.

Embodiment 132: The electrode of embodiment 130 or 131, wherein the target binding molecule is a receptor, a ligand for a receptor, an antibody, antigen binding fragment of an antibody, an antigen, an enzyme or a nucleic acid.

Embodiment 133: The electrode of any one of embodiments 130-132, wherein the target binding molecule is an antibody, an antigen binding fragment of an antibody or an antigen.

Embodiment 134: The composition of any one of the preceding embodiments, wherein the composition comprises an anti-microbial agent.

Embodiment 135: The composition of embodiment 130, wherein the anti-microbial agent is an anti-bacterial agent, anti-fungal agent or anti-viral agent.

Embodiment 136: The composition of embodiment 130 or 131, wherein the anti-microbial agent is an anti-bacterial agent.

Embodiment 137: The composition of embodiment 128, wherein the anti-bacterial agent is selected from the group consisting of macrolides or ketolides such as erythromycin, azithromycin, clarithromycin, and telithromycin; beta-lactams including penicillin, cephalosporin, and carbapenems such as carbapenem, imipenem, and meropenem; monolactams such as penicillin G, penicillin V, methicillin, oxacillin, cloxacillin, dicloxacillin, nafcillin, ampicillin, amoxicillin, carbenicillin, ticarcillin, meziocillin, piperacillin, azlocillin, temocillin, cepalothin, cephapirin, cephradine, cephaloridine, cefazolin, cefamandole, cefuroxime, cephalexin, cefprozil, cefaclor, loracarbef, cefoxitin, cefmetazole, cefotaxime, ceftizoxime, cefiriaxone, cefoperazone, ceftazidime, cefixime, cefpodoxime, ceftibuten, cefdinir, cefpirome, cefepime, cefadroxil, ceftriaxone, ceftobiprole and astreonam; quinolones such as nalidixic acid, oxolinic acid, norfloxacin, pefloxacin, enoxacin, ofloxacin, levofloxacin, ciprofloxacin, temafloxacin, lomefloxacin, fleroxacin, grepafloxacin, sparfloxacin, trovafloxacin, clinafloxacin, gatifloxacin, moxifloxacin, sitafloxacin, ganefloxacin, gemifloxacin and pazufloxacin; antibacterial sulfonamides and antibacterial sulphanilamides, including para-aminobenzoic acid, sulfadiazine, sulfisoxazole, sulfamethoxazole and sulfathalidine; aminoglycosides such as streptomycin, neomvcin, kanamycin, paromycin, gentamicin, tobramycin, amikacin, netilmicin, spectinomycin, sisomicin, dibekalin and isepamicin; tetracyclines such as tetracycline, chlortetracycline, demeclocycline, minocycline, oxytetracycline, methacycline, doxycycline; rifamycins such as rifampicin (also called rifampin), rifapentine, rifabutin, bezoxazinorifamycin and rifaximin; lincosamides such as lincomycin and clindamycin; glycopeptides such as vancomycin and teicoplanin; streptogramins such as quinupristin and daflopristin; oxazolidinones such as linezolid; polymyxin, colistin and colymycin; trimethoprim, bacitracin, and phosphonomycin.

Embodiment 138: The composition of embodiment 130 or 131, wherein the anti-microbial agent is an antifungal agent.

Embodiment 139: The composition of embodiment 138, wherein the antifungal agent is selected from the group consisting of azoles (e.g., barleyconazole, butoconazole, clortrimazole, econazole, fluconazole, isavuconazole, itraconazole, ketoconazole, miconazole, oxyconazole, posaconazole, ravuconazole, saperconazole, sulconazole, tercocnazole, tioconazole, voriconazole, and ciclopirox), polyenes (e.g., natamycin, lucensomycin, nystatin, amphotericin B, etc.), echinocandins (e.g., Cancidas), pradimicins (e.g., beanomicins, nikkomycins, sordarins, allylamines, etc.), Triclosan, Piroctone, fenpropimorph, terbinafine, cyclopyroxolamine, flucitocin, griseofulvin haloprozin, tolnaftate, naphthypine, hydrochloride, morpholine, butenapin, undecylenic acid, propionic acid, and derivatives and analogs thereof.

Embodiment 140: The composition of embodiment 130 or 131, wherein the anti-microbial agent is an antimicrobial peptide or polymer.

Embodiment 141: The composition of embodiment 130 or 131, wherein the anti-microbial agent is a metal particle.

Embodiment 142: The composition of embodiment 141, wherein anti-microbial agent is titanium oxide, copper, or silver nanoparticles.

Embodiment 143: The composition of any one of embodiments 85-142, wherein the composition further comprises a therapeutic agent, e.g., anti-inflammatory drugs sirolimus, everolimus, biolimus (A9), zotarolimus (ABT-578), tacrolimus, and pimecrolimus, genistein, steroids (dexamethasone, prednisolone, methylprednisolone and hydrocortisone), fluocinolone acetonide, hormones etc.

Embodiment 144: The composition of any one of embodiments 85-143, wherein the composition further comprises a polymer.

Embodiment 145: The composition of embodiment 144, wherein the polymer is a water miscible polymer.

Embodiment 146: The composition of embodiment 144 or 145, wherein the polymer is a degradable polymer

Embodiment 147: The composition of any one of embodiments 144-146, wherein the polymer is selected from the group consisting of poly(N-isopropyl acrylamide) (PNIPAAm), polyethylene glycol (PEG), alginate, polytetrafluoroethylene (PTFE), polyacetylene (PA), polyaniline (PANI), polypyrrole (PPy), polythiophene (PTH), poly(para-phenylene) (PPP), poly(phenylenevinylene) (PPV), and polyfuran (PF).

Embodiment 148: A surface comprising an antifouling coating layer on at least a part of the surface, wherein the coating layer comprises a composition of any one of the preceding embodiments.

Embodiment 149: The surface of embodiment 148, wherein the coating layer is directly or indirectly connected with an electrode.

Embodiment 150: The surface of any one of embodiments 148-149, wherein the coating layer is adapted for contact with an analyte or a sample comprising an analyte.

Embodiment 151: The surface of any one of embodiments 148-150, wherein the coating layer is porous.

Embodiment 152: The esurface of any one of embodiments 148-151, wherein the coating layer has a porosity of about 5% to about 95%.

Embodiment 153: The surface of any one of embodiments 148-152, wherein the coating layer comprises macropores (e.g., pores having a diameter of from about 0.1 μm to about 10 μm such as from about 0.5 μm to about 5 μm or from about 1 μm to about 3 μm).

Embodiment 154: The surface of any one of embodiments 148-153, wherein the coating layer comprises mesopores (e.g., pores having a diameter from about 5 nm to about 99 nm).

Embodiment 155: The electrode of any one of embodiments 148-154, wherein the coating layer comprises nanopores (e.g., pores having a diameter of from about 0.1 nm to about 4.5 nm).

Embodiment 156: The surface of any one of embodiments 148-155, wherein the coating layer has a thickness from about 2 nm to about 100 μm.

Embodiment 157: The surface of any one of embodiments 148-156, wherein surface is a conductive substrate.

Embodiment 158: The surface of embodiment 157, wherein the conductive substrate comprises gold, silver, copper, platinum, aluminum, stainless steel, tungsten, indium tin oxide, titanium, lead, nickel, palladium, zirconium, niobium, tantalum, chromium, molybdenum, manganese, rhenium, ruthenium, rhodium, iridium, silicon, polyimide, parylene, benzocyclobutene, carbon, graphite, or any combination thereof.

Embodiment 159: The surface of embodiment 157 or 158, wherein the conductive substrate comprises a flexible substrate.

Embodiment 160: The electrode of embodiment 159, wherein the flexible substrate comprises polyethylene terephthalate, polyethylene naphathalate, polyimides, polymeric hydrocarbons, celluloses, plastics, polycarbonates, polystyrenes, or any combination thereof.

Embodiment 161: Use of an electrode of any one of embodiments of claims 1-80 or sensor of any one of embodiments 81-84 for detecting a target analyte in a sample.

A method for detecting a target analyte in a sample, the method comprising:
a. contacting a sample suspected of comprising a target analyte with an electrode of any one embodiments 1-161; and
b. detecting oxidation or reduction of the target analyte by the oxidoreductase present in or on the antifouling coating.

Embodiment 162: The method of embodiments 161, wherein said detecting oxidation or reduction of the substrate comprises applying a voltage to the electrode.

Embodiment 163: The method of any one of embodiments 161-162, wherein said detecting oxidation or reduction of the substrate comprises measuring a current generated from electrode.

Embodiment 164: The method of any one of embodiments 162-163, wherein said detecting oxidation or reduction of the substrate comprises detecting oxidation or reduction of the redox mediator present in or on the antifouling coating.

Embodiment 165: The method of any one of embodiments 162-164, wherein the target analyte is a peptide, a polypeptide, a peptidomimetic, a nucleic acid, an oligosaccharide, a polysaccharide, an amino acid, nucleoside, a nucleotide, a carbohydrate, a lipid, a peptidoglycan, a cell, microbial matter, an antigen, a lipid, a steroid, a hormone, a lipopolysaccharide, an endotoxin, a therapeutic agent, a lipid-binding molecule, a cofactor, a small molecule, a toxin, a biological threat agent (e.g., spore, viral, cellular and protein toxin), or any combination thereof.

Embodiment 166: The method of any one of embodiments 162-165, wherein the target analyte is a protein, an antibody, an antigen binding fragment of an antibody, an antigen, a hormone, or a metabolite.

Embodiment 167: The method of any one of embodiments 162-166, wherein the target analyte is a tumour marker or a clinical chemistry target.

Embodiment 168: The method of any one of embodiments 162-167, wherein the sample is a biological sample (e.g., blood, saliva, amniotic fluid, sputum, urine, semen, cerebrospinal fluid, bronchial aspirate, perspiration, mucus, liquefied stool sample, synovial fluid, lymphatic fluid, tears, tracheal aspirate, lactation product, and any combination thereof).

Embodiment 169: The method of any one of embodiments 162-168, wherein the sample is a food, an ingredient for preparing a food, poultry, meat, fish, beverage, or dairy product.

Embodiment 170: The method of any one of embodiments 162-169, wherein the sample is a non-biological sample (e.g., water, salt water, pond water, river water, reservoir water, brine, drinking water, industrial water, brown water, waste water, sewerage, soil, and mixtures thereof.

Embodiment 171: The method of any one of embodiments 162-170, wherein the sample is pre-processed prior to contacting with the electrode.

Embodiment 172: A kit comprising an electrode, sensor, composition or surface of any one of the preceding embodiments.

### Some Selected Definitions

For convenience, certain terms employed herein, in the specification, examples and appended claims are collected herein. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

As used herein the term “comprising” or “comprises” is used in reference to compositions, methods, and respective component(s) thereof, that are essential to the invention, yet open to the inclusion of unspecified elements, whether essential or not. In other words, except where the context requires otherwise, the term “comprise” and variations of the term, such as “comprising”, “comprises” and “comprised”, are not intended to exclude further additives, components, integers or steps.

The singular terms “a,” “an,” and “the” include plural referents unless context clearly indicates otherwise. Similarly, the word “or” is intended to include “and” unless the context clearly indicates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

As used herein, the term “binding” or “bound” generally refers to a reversible binding of one molecule to molecule via, e.g., van der Waals force, hydrophobic force, hydrogen bonding, and/or electrostatic force. The binding interaction between two molecules can be described by a dissociation constant (K_{d}) or association constant (K).

Specific elements of any of the disclosed embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

### EXAMPLES

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

### Example 1: An Enzyme-Based Electrochemical Sensor with Enhanced Sensitivity and Specificity Based on Incorporation of a Redox-Active Nanostructured Antifouling Coating for Metabolomic and Diagnostic Applications

The continuous monitoring of metabolites, such as glucose and lactate, has been typically limited due to the cross-reactivity of the off-target molecules and instability of the signals in diagnostic devices. Presented here, is a method to measure the concentration of metabolites such as glucose (a model metabolite) using a novel electrochemical platform based on a nanostructured redox-embedded antifouling coating for continuous monitoring of metabolites over time. This rapid, sensitive, low-cost, portable method is desirable as a readout for metabolite-related disease diagnostics. It can be readily interfaced with portable potentiostats without requiring a trained user or specialized facilities. Furthermore, the technology can be used for real-time and multiplex detection of metabolites in different biological samples such as Blood, Saliva, and cell culture. It can be multiplexed with affinity-based electrochemical sensors that use similar antifouling coatings to provide multi-parameter readouts.

### Background

An imbalance of metabolites is considered one of the most severe threats to human health and is related to the high risks of several serious illnesses. The increasing growth of metabolomic analyses over the last decade shows the trend for developing new diagnostic methods based on detection of changes in levels metabolites that are indicative of various disease states (e.g., diabetes, cancer, etc.). Therefore, fast and reliable detection of these biomarkers at low cost can be of great value for disease diagnosis in hospitals and at the point-of-care (POC).

Metabolite concentrations can be influenced by factors of intrinsic nature (biochemical reactions) or exogenous origin (diet or medication). Thus, continuous measurement of metabolite levels in biological samples, such as blood, saliva, and urine, is of central importance in clinical diagnostics. However, real-time measurement of metabolite concentrations in complex biological fluids presents considerable challenges because non-specific binding of other materials in these fluids can decrease sign specificity and sensitivity over time.

### Invention

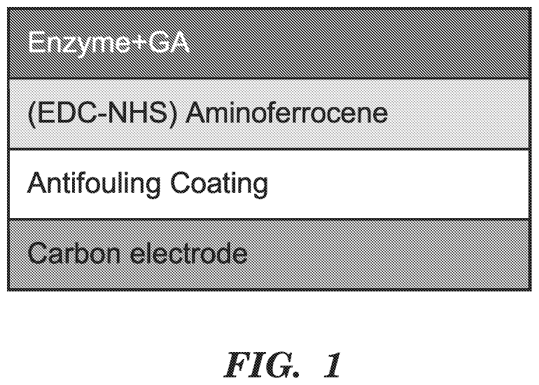

Here the inventors report a method to develop enzymatic sensors to precisely measure the metabolite levels in complex biological fluids over time using a novel nanostructured electrochemical biosensor that contains screen-printed electrodes (SPE) overlaid with a nanocomposite coating modified with a mediator (Aminoferrocene) to lower the working oxidation potential thereby, preventing background electrochemical signal from interfering molecules (e.g., dopamine, uric acid) (FIG. 1). Such technology can further enhance function for implantable sensors.

The carbon screen-printed electrode is chemically modified by coating an antifouling nanoporous composite containing graphene, glutaraldehyde, BSA, and a reversible redox molecular system that is sensitive to H₂O₂ (e.g., ferrocene, hexaammineruthenium) which is then linked to corresponding oxidase enzymes that convert target metabolites to H₂O₂, enabling the direct and continuous detection of complex biological samples like blood and saliva.

The detailed procedure is presented below:
1. Prepare 5 mg/mL recombinant human albumin (rHA)/bovine serum albumin (BSA) stock solution in 10 mM PBS (pH 7.1-7.5) by measuring 5 mg of rHA/BSA dissolved in 1 mL of PBS solution. This can be stored in the fridge (4-8° C.) until further use.
2. Measure 8 mg of pentaamine functionalized reduced graphene oxide (prGOx) and prepare a solution of 8 mg/mL prGOx solution using 5 mg/mL rHA/BSA solution dissolved in 10 mM PBS (pH 7.1-7.5) solution. Mix the solution in a vortex mixture for 5 sec. Sonicate the mixture for 1 hour by keeping in a centrifuge tube. Make sure that the probe is well within the solution. (1 s on/off cycles for 30 min).
3. Heat the solution for 5 min at 105.5° C. to denature the protein. Thereafter, spin at 16,200 rpm for 15 min to remove prGOx aggregates.
4. Carefully remove the supernatant and add glutaraldehyde in the ratio of 1:70, and vortex the solution.
5. Clean the SPE carbon electrode with Ethanol, polish it with Chemwipe, add 70 μL over the SPE electrode, and incubate overnight.
6. Dip the chip in Petri dish containing PBS and rinse for 10 min.
7. Prepare EDC/NHS solution by adding 77 mg of ethyl(dimethylaminopropyl) carbodiimide (EDC) and 23 mg of N-hydroxysuccinimide ester (NHS) to 1 ml of MES buffer (50 mM MES buffer, pH 6.2) and incubate 70 μL of freshly prepared EDC/NHS solution on every chip for 30 min.
8. Wash the chip briefly with Milli Q Water and dry.
   a. Add 40 μL of AminoFerrocene dissolved in PBS (100 mg/ml) to the electrode and incubate for 45 minutes.
9. Rinse the SPE with PBS by adding PBS to the Petri dish with chips.

Add 5 μL of a mixture of glucose oxidase (80 mgmL-1) and Gluturaldehyde (5%) in a ratio of 1:1 on the working electrode and let it incubate overnight at 4-8 degrees

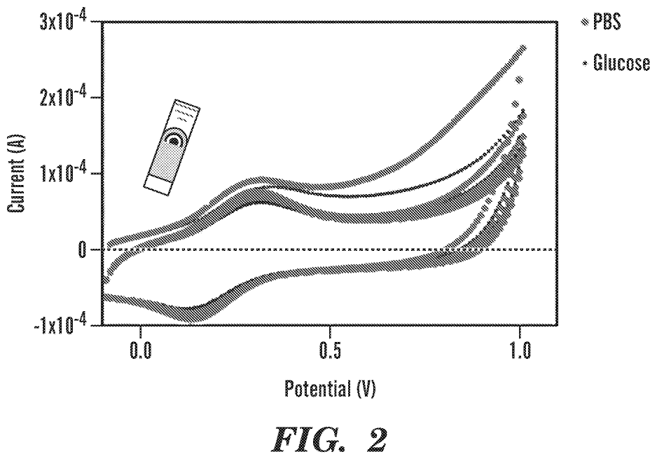

The CV responses for the redox-embedded antifouling coating (FIG. 2) were recorded in PBS without glucose at scan rates of 200 mV/s. The oxidation and reduction peaks at 0.1 and 0.3 are attributed to the presence of redox molecules in the coating and demonstrate that the ferrocene can be reversibly oxidized from ferrocene (Fe²⁺) to the ferrocenium ion (Fe³⁺). The consistency of the signal after a few cycles shows the stability of the redox molecule in the film.

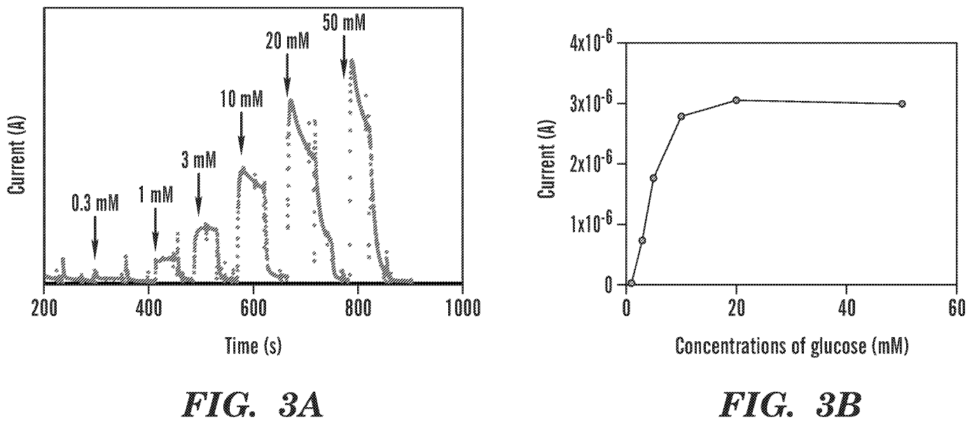

FIGS. 3A and 3B demonstrate the amperometric response of the biosensor. The electrochemical measurements were performed in PBS with different concentrations of glucose. The calibration curve was plotted by picking the signal at 20 seconds after exposure to the glucose to the biosensor.

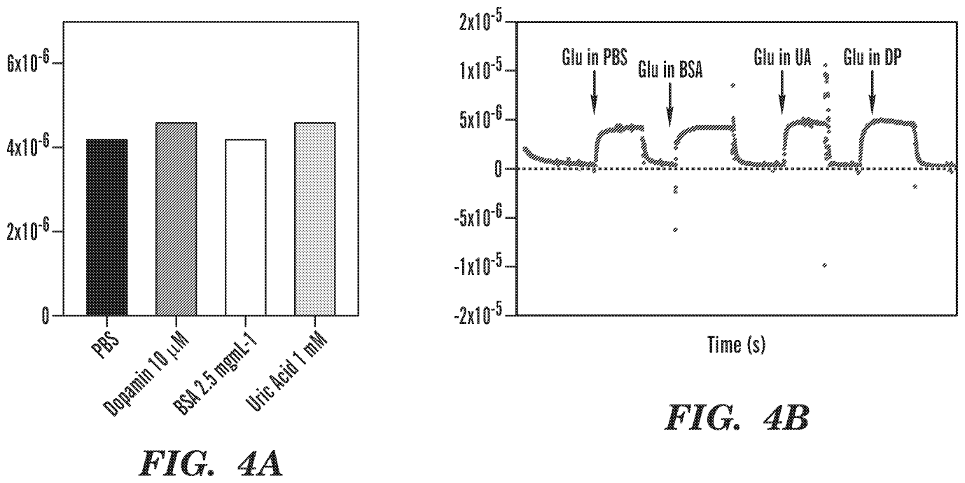

The cross-reactivity of the biosensor was evaluated by exposing the biosensor to 5 mgmL⁻¹ glucose in PBS, 2.5% BSA, Uric acid, and Dopamine (FIG. 4).

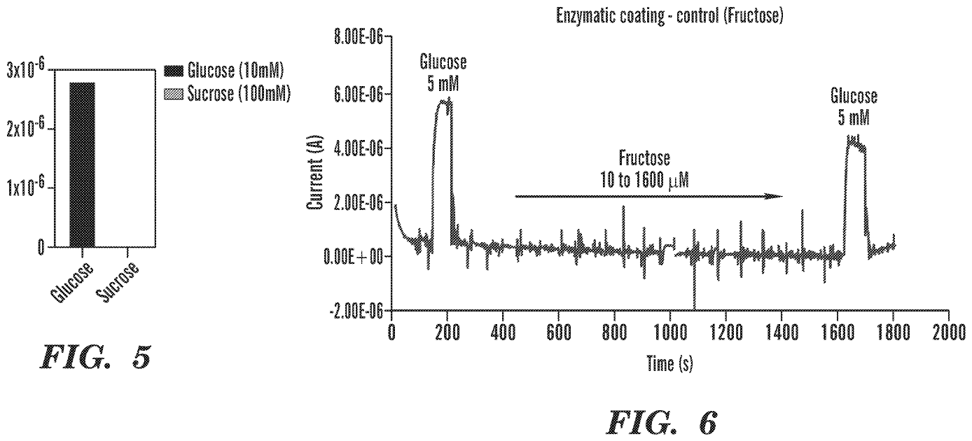

To evaluate the specificity of the biosensor, the biosensor was exposed to high concentrations of Sucrose (100 mM), and the results were compared with the response of the biosensor to 10 mM glucose FIG. 5.

The sensor was further challenged with fructose for specificity. As seen in FIG. 6, fructose failed to show any response from 10-1600 μM range, while 5 mM glucose gave a specific signal before and after exposing the sensor to fructose.

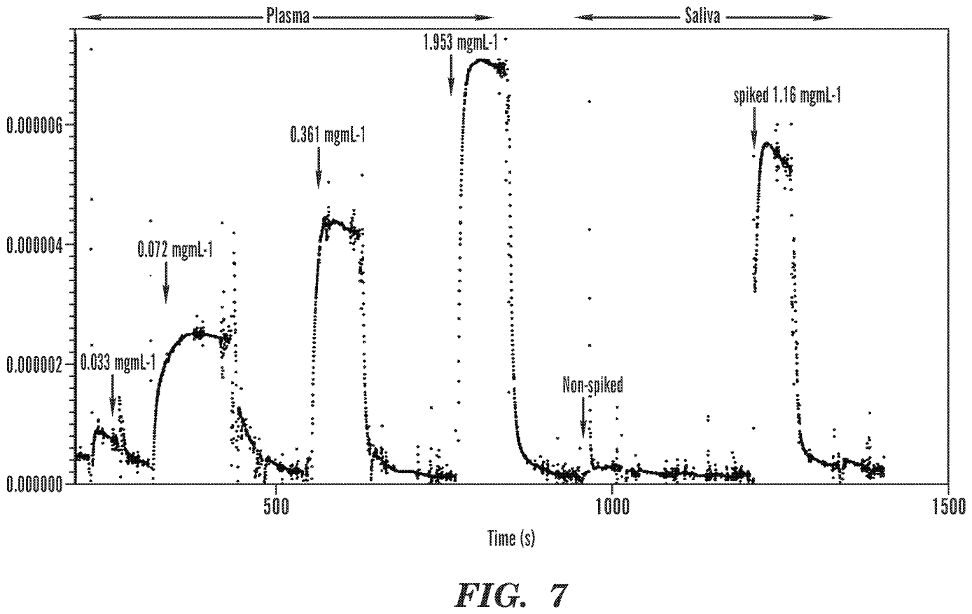

The biosensor's function was evaluated by exposing the biosensor to plasma and saliva samples containing different concentrations of glucose levels (FIG. 7). The biosensor showed accurate responses to the presence of the target molecule in complex biological fluids. The sensor's response is real-time and completely dependent on glucose concentration in the biological sample. The detection window of the biosensor is in the glucose range in both normal and patient samples. This linear range can be tailored by changing the biosensor's film properties.

### Example 2: Redox-Modified Nanostructured Electrochemical Surfaces for Continuous Determination of Glucose in Complex Biological Matrices

Continuous glucose monitoring is valuable for people with diabetes but faces limitations due to enzyme-electrode interactions affecting sensor sensitivity. Biofouling from biological samples also hinders performance. Described herein is an enzyme-based electrochemical system with a unique nanocomposite coating incorporating the redox molecule aminoferrocene (NH₂-Fc). This coating enhances stability via electroactivity and reduction of nonspecific binding, demonstrated through cyclic voltammetry. This approach enables real-time glucose detection via chronoamperometry, with a calculated linear range of 0.5 to 20 mM and a 1 mM detection limit. Validated with plasma and saliva, this platform shows robust metabolite detection in clinical and research contexts. This versatile platform can be applied to accurately monitor wide range of metabolites in various biological matrices improving clinical outcomes of patients.

Diabetes prevalence has drastically increased worldwide, with more than half a billion people affected by this condition¹. The current prevalence rate of 6.1% puts diabetes as one of the leading causes of death globally^{1,2}, and diabetes is considered a significant contributor to other chronic diseases, including heart attack, stroke, and kidney disease^{2, 3}. Elevated levels of glucose in the blood, if not recognized early, are extremely life-threatening, yet 23% of adults living with diabetes remain undiagnosed in North America⁴. Therefore, accurate, reliable, and timely monitoring of glucose from human samples (e.g., saliva, plasma, blood, etc.) is essential for the prevention and management of diabetes and its related complications. To date, although many glucose biosensors based on different transduction principles have been developed, electrochemical-based glucose biosensors utilizing enzymatic sensing dominate the market^{3, 5, 6}. Such devices harness the inherent electrochemical properties of metabolites, such as their reduction or oxidation capabilities, to design systems that can rapidly quantify target analytes from complex biological fluids with high reliability. In addition, most glucose biosensors rely on enzymatic oxidation of glucose mainly due to the high selectivity and stability of glucose oxidase (GluOx)⁶⁻⁸, which renders these biosensors with better performance than current technologies in biological fluids and extended shelf life.

Currently, rapid quantification of glucose levels from finger-prick blood samples is achieved using a glucometer, a handheld biosensing device that enables regular monitoring at home. However, several factors, such as deteriorated test strips, reader's imprecision, bias, improper storage, interference from multiple medications, and environmental conditions, may impact the reliability of these blood glucose measurements⁹⁻¹². Besides, the sample site may become painful, and wounds from finger-pricks are at risk of infection due to the requirement for repeated testing^{3, 12}. Also, these devices utilize a single-point measurement mode which is not practical for continuous monitoring; the testing frequency does not provide any information about the occurrences of hypo/hyperglycemic levels required to improve glycemic control in diabetes patients^{10, 11}. Continuous glucose monitors (CGM) that relay glucose levels in real-time have been developed to overcome these limitations. CGMs can be life-changing as they facilitate more personalized insight into glucose level changes during the day, offering patients the power to make more informed treatment decisions and prevent episodes of hypo/hyperglycemia^{11, 13}. Although many companies have successfully created and commercialized several CGMs, their application for long-term monitoring, to date, remains restricted due to inaccuracy at low glucose concentrations, high cost, short duration of single implantation, and biocompatibility issues^{13, 14}.

Biosensors exposed to physiological environments for extended periods may exhibit high background current or signal drift that could compromise detection sensitivity and analytical accuracy¹². This is because these sensors are constructed using materials that are prone to passivation by fouling agents (i.e., proteins, amino acids, lipids, etc.) that form an impermeable layer on the electrode surface, affecting charge transfer kinetics between the target analyte and the electrode surface¹⁵. This effect discourages the application of biosensors for continuous monitoring since fouling degrades the sensor surface, resulting in a low signal-to-noise ratio and reduced sensitivity^{12, 15}. As described herein this crucial challenge is addressed by a novel nanocomposite coating based on glutaraldehyde (GA) crosslinking of conductive nanomaterials, such as reduced graphene oxide (prGOx), dissolved with bovine serum albumin (BSA) in PBS^{16, 17}. This nanocomposite formulation not only reduces fouling by creating a more hydrophilic surface at the electrode/electrolyte interface, preventing non-specific adsorption, but also offers enhanced electroconductive properties promoting their electrochemical performance in biological fluids. The inventors successfully demonstrated the potential of this antifouling coating on an electrochemical sensor to detect various biomolecules, including IL-6, PCT, CRP, SARS COV NS1, GFAP, and NFL in different biological matrices¹⁸-21. In this work, the potential of a prGOx/BSA/GA nanocomposite coating towards the construction of a glucose biosensor to address the electrode fouling challenges observed in enzyme-based continuous monitoring systems is demonstrated.

In label-based electrochemical systems, detection is usually achieved by diffusion of redox molecules, such as potassium ferro/ferricyanide, from electrolyte to electrode surfaces²². Though these biosensors are robust and reliable, sometimes redox probes can contaminate electrode systems, giving rise to non-specific signals, which could be overcome by functionalizing a stable redox-active functional molecule, such as a ferrocene derivative^{23, 24}. Specific recognition between the capture molecule and target analyte will impede the charge transfer between the immobilized redox probe and electrode surface, which could be leveraged to analyze biosensor performance. Several research groups have developed biosensing platforms modified with redox mediators to detect various enzymes, including glucose²²⁻²⁴, proteins, and nucleic acids²⁵. However, these platforms do not address the challenges related to fouling that should be eliminated to translate these devices toward successful commercialization.

Functionalization of the prGOx/BSA/GA nanocomposite coating with a redox-based molecule that has low redox potential and good electrochemical stability can offer a multifaceted biosensing platform for continuous monitoring of glucose in biological fluids. Therefore, described herein, is a facile fabrication of a redox-embedded nanocomposite sensor and its application for glucose detection in various biological matrices, saliva, and plasma. The redox molecule, aminoferrocene (NH₂-Fc), is a ferrocene (Fc) derivative with NH₂ substitution, covalently coupled to prGOx/BSA/GA. NH₂-Fc undergoes reversible oxidation and reduction reactions (Fc↔Fc⁺) and exhibits good electrochemical behavior that favors reagentless determination of target analytes. Here, NH₂-Fc efficiently mediates enzymatic oxidation of glucose via immobilized glucose oxidase and retains the activity of an enzyme-immobilized glucose biosensor. Also, the low redox potential (~0.2 V) of NH₂-Fc favors reducing the background signal from other interfering electroactive species present in biological samples. Through rigorous experimental evaluations and comparative analysis, we demonstrate for the first time, the potential of a NH₂-Fc functionalized prGOx/BSA/GA nanocomposite-coated electrochemical sensor towards glucose detection with high sensitivity and selectivity.

### Materials and Methods

### Nanocomposite Deposition

Screen-printed carbon electrodes (SPCEs) (DropSens, Metrohm, USA) were cleaned with ethanol (Sigma Aldrich, USA), polished with Kimwipes, and washed with deionized water. SPCEs were dried, and oxygen plasma-treated at 0.5 mbar and 50% power for 8 min (Zepto Diener Plasma, Diener Electronics, Germany). The antifouling nanocomposite coating consisting of pentaamine-functionalized reduced graphene oxide (prGOx; Sigma Aldrich, USA) and bovine serum albumin (BSA; IgG-Free, Protease-Free; Jackson ImmunoResearch, USA) was prepared in 10 mM phosphate-buffered saline (PBS, pH 7.4) as reported^{16, 17}. Briefly, the prepared solution was subjected to tip sonication with Is ON/OFF pulse at 50% amplitude for 30 min (Q125, QSonica LLC, USA), followed by heating at 105° C. for 5 min. The protein-denatured solution was centrifuged at 16.2 rcf for 15 min to remove excess prGOx/BSA aggregates. A mixture of prGOx/BSA supernatant solution and 70% glutaraldehyde (GA; Sigma Aldrich, USA) was prepared in the ratio of 69:1, applied over plasma-treated SPCEs, and incubated overnight in a humidity chamber. The prGOx/BSA/GA nanocomposite-deposited SPCEs were washed the following day with PBS on a shaker at 500 rpm for 10 min and dried using a slide spinner.

### Fabrication of the Redox-Functionalized Glucose Sensor

The surface of nanocomposite-deposited SPCEs was activated using carbodiimide chemistry, wherein 400 mM of 1-ethyl-3-(3-dimethylaminopropyl) carbodiimide hydrochloride (EDC; Thermo Fisher Scientific, USA) and 200 mM of N-Hydroxysuccinimide (NHS; Sigma Aldrich, USA) dissolved in 50 mM 2-(N-morpholino) ethanesulfonic acid (MES; Thermo Fisher Scientific, USA) buffer at pH 6.2 was added to the sensor and incubated for 30 min at room temperature. SPCEs were then rinsed with MilliQ water and dried before the addition of redox molecules. Aminoferrocene (NH₂-Fc; Sigma Aldrich, USA) dissolved in PBS at 1 mg/ml concentration was added to the working electrode surface and incubated for 45 min., then rinsed with PBS by agitation at 500 rpm for 5 min and dried. Following the functionalization of NH₂-Fc, the working electrodes were selectively spotted with 2 μL of an enzymatic solution obtained by mixing glucose oxidase at 80 mg/mL with GA in a 1:1 v/v ratio. Glucose oxidase immobilized SPCEs were dried overnight at 4° C. to let the enzyme composition adhere to the prGOx/BSA/GA nanocomposite-coated surfaces. When the electrode surface was completely dry, it was washed gently with PBS and kept stored at 4° C. until use.

### Electrochemical Measurements

All electrochemical measurements were carried out on an Autolab PGSTAT128N potentiostat (Metrohm, USA). Cyclic voltammetry (CV) was used to electrochemically characterize the NH₂-Fc functionalized prGOx/BSA/GA nanocomposite coating and to determine the oxidation/reduction potential of NH₂-Fc. The potential for chronoamperometry measurements used for glucose quantification was obtained from CV measurements. Calibration curves were built for glucose concentrations between 0 mM and 50 mM based on chronoamperometry.

### Sample Collection

In this study, saliva and plasma samples were collected from a volunteer following ethical guidelines and informed consent. Saliva samples were obtained using passive drool collection, while plasma samples were collected through venipuncture and processed to obtain the plasma fraction. Both saliva and plasma samples were stored at −80° C. Prior to electrochemical analysis, samples were thawed at room temperature and thoroughly mixed. Samples were tested both in their natural state and spiked with glucose, each undergoing three tests for accuracy and reliability.

### Results

Fabrication of the Nanocomposite Interface with Confined Redox Probes

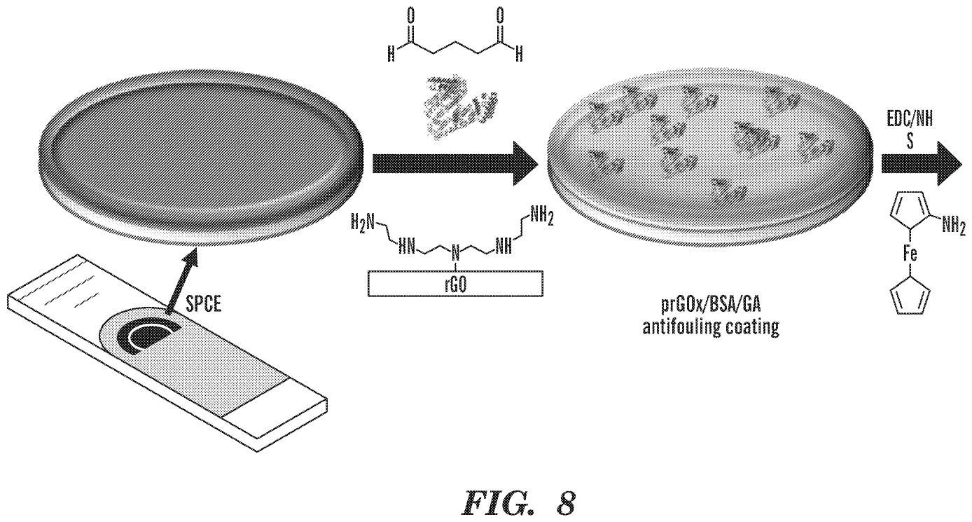

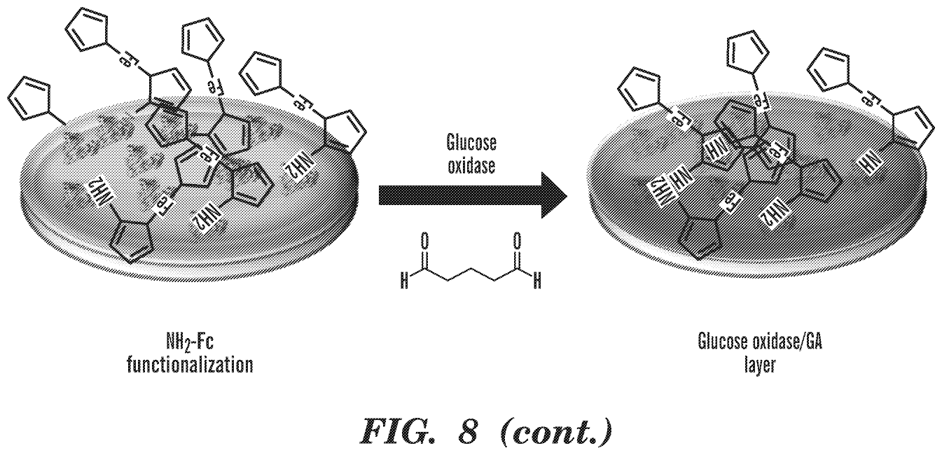

FIG. 8 demonstrates facile fabrication of the NH₂-Fc functionalized glucose biosensor interface which is achieved in three steps. First, the prGOx/BSA/GA nanocomposite coating is drop-cast onto the SPCE surface. The conductive material (prGOx) facilitates electron transfer and improves the electroactive surface area, BSA offers stability, and GA prevents biofouling. Second, the redox mediator, NH₂-Fc, is covalently functionalized onto the nanocomposite coating through EDC/NHS chemistry. The carbonyl carbon of the NHS ester group undergoes nucleophilic substitution on reaction with the amine group of NH₂-Fc derivative, resulting in a stable amide bond. Finally, an enzyme layer is coated onto the NH₂-Fc modified SPCE by drop-casting a solution containing glucose oxidase and GA, and then dried. The GA-crosslinked enzyme layer offers selective permeability for glucose and provides long-term stability for glucose oxidase, in addition to its antifouling properties in biological fluids.

### Electrochemical Characterization of the Redox Immobilized Glucose Sensor

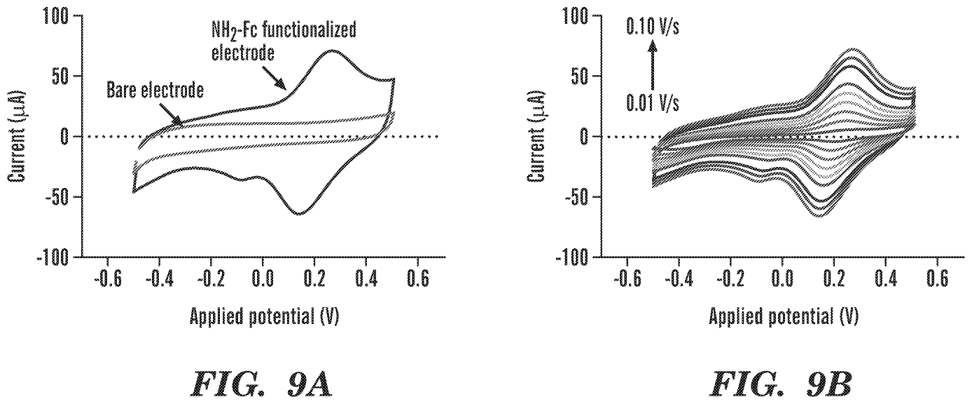

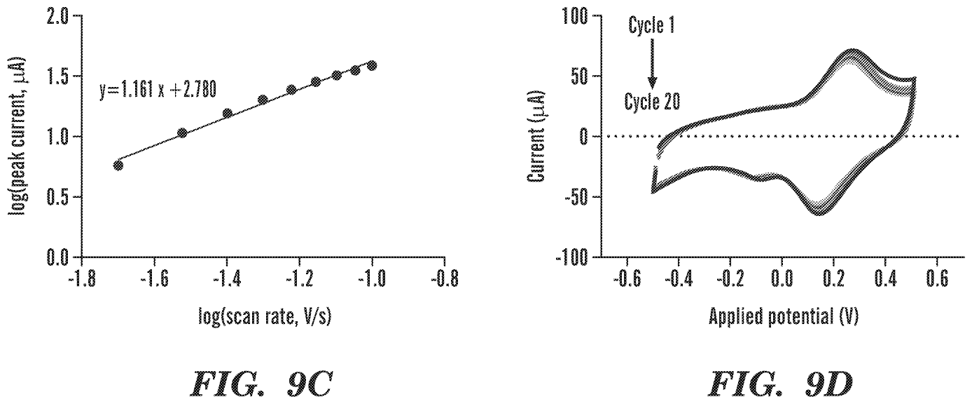

Understanding the charge transfer kinetics of NH₂-Fc functionalized electrode surfaces is crucial to determining their electrochemical performance. Biomolecular binding interactions at the electrode/electrolyte interfaces alter the redox-based charge transfer processes, which can be utilized to design an efficient electrochemical biosensor for various applications. Here, the redox behavior of NH₂-Fc modified and bare prGOx/BSA/GA nanocomposite coated SPCEs was obtained using CV measurements in PBS. The CV of NH₂-Fc modified SPCE revealed a defined quasi-reversible oxidation and reduction peak at 0.23V and 0.15V, respectively, with a peak separation of 80 mV. In contrast, the bare nanocomposite-coated SPCE (FIG. 9A) showed no well-defined peaks. This is indicative of the oxidation and reduction of an Fc derivative in NH₂-Fc during the electrochemical processes, and obtained peaks were consistent with the redox potential of NH₂-Fc, confirming the successful functionalization of NH₂-Fc onto the prGOx/BSA/GA nanocomposite coating.

The electrochemical oxidation and reduction of NH₂-Fc functionalization were further evaluated with scan rates ranging from 10 mV/s to 100 mV/s. The voltammograms in FIG. 9B show a characteristic CV curve with increasing redox peak currents for NH₂-Fc with increasing scan rate. The plot for log of scan rate versus log of redox peak current exhibited a linear relationship with a slope of 1.1 which is strongly indicative of NH₂-Fc functionalization contributing towards surface-controlled electrochemical behavior (expected slope is 1.0) (FIG. 9C).

To further elucidate the stability of NH₂-Fc functionalization, the prepared prGOx/BSA/GA nanocomposite-coated sensor was subjected to CV measurements in PBS by cycling the potential between-0.5V to +0.5V at 100 mV/s for 20 cycles. No significant change in peak current with increasing scan cycles was observed (FIG. 9D), suggesting the long-term electrochemical stability of the NH₂-Fc functionalized prGOx/BSA/GA nanocomposite coating.

### Glucose Detection in a Standard Solution

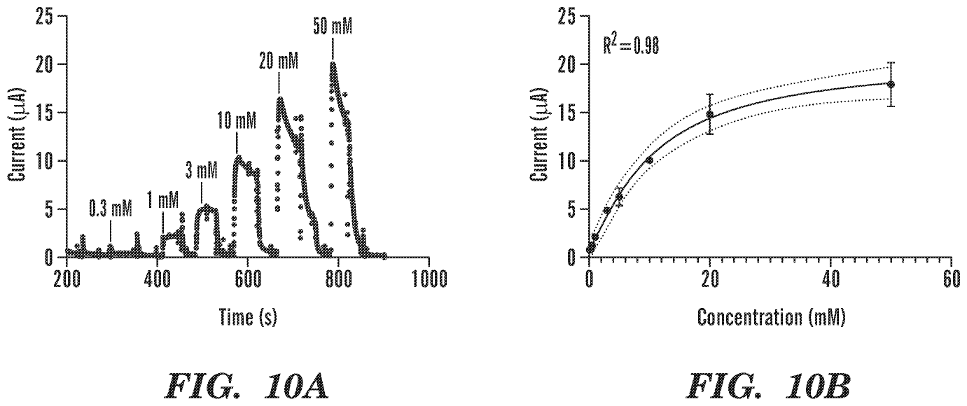

The sensor response to glucose was measured for glucose concentrations between 0.1 mM to 50 mM using chronoamperometry. Initially, we investigated chronoamperometry measurements at different potentials (0, 0.2 V) to determine the signal sensitivity. Glucose detection measurements were subsequently performed using chronoamperometry at 0.2V based on these results. FIG. 10A shows changes in the current response of the NH₂-Fc functionalized prGOx/BSA/GA sensor when exposed to varying glucose concentrations in PBS. A calibration curve was generated by plotting the average of the current response 20 sec after glucose exposure. The sensor exhibits a linear behavior between 0 mM and 20 mM glucose concentrations with a detection limit of 1 mM (FIG. 10B).

### Specificity and Selectivity

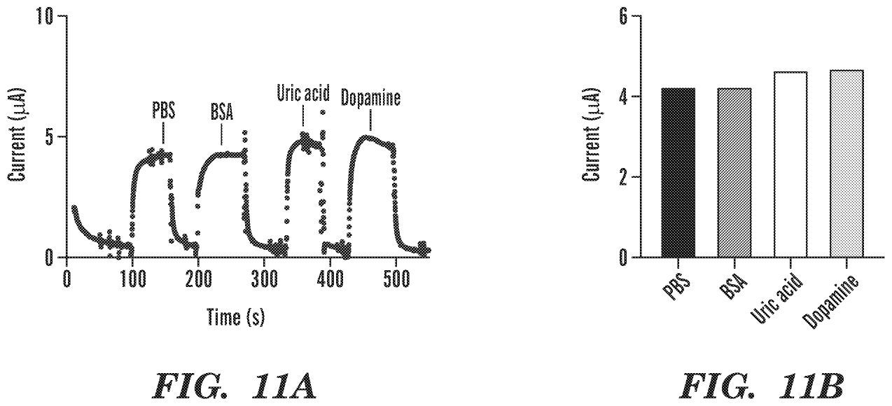

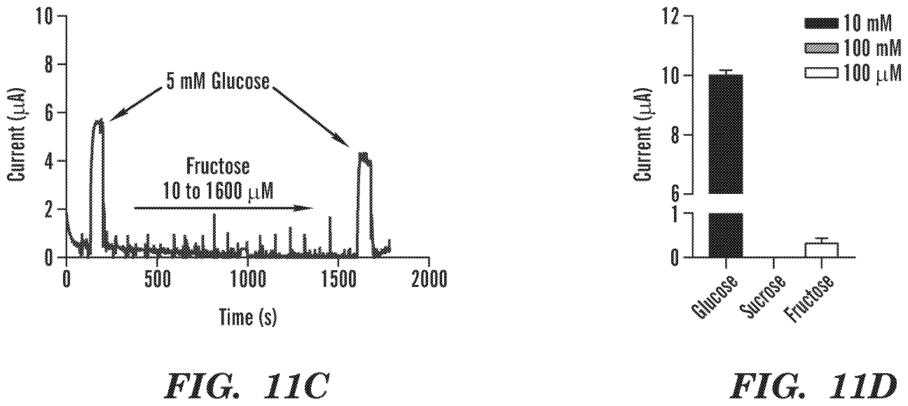

Biological fluids contain a plethora of metabolites and electrolytes that could potentially interfere with glucose measurements, giving rise to non-specific signal responses. Therefore, the specificity and selectivity of our developed glucose sensor were determined in the presence of various interfering electroactive constituents. The NH₂-Fc functionalized glucose sensor was first exposed to physiologically relevant concentrations of potentially interfering substances, such as BSA (2.5 mg/mL), uric acid (1 mM), and dopamine (10 μM) separately spiked in a 5 mM glucose solution. The NH₂-Fc functionalized glucose sensor responded selectively toward glucose detection compared to other interferents (FIG. 11A). Metabolites such as uric acid and dopamine have positive redox potentials, however, no change in electrochemical signal response was observed with these potential interferants (FIGS. 11A and 11B), and the current response remained almost the same as the test solution spiked with only glucose. This suggests no electrochemical interference from such metabolites, and the electrochemical signal obtained is from the of oxidation/reduction of an Fc derivative in NH₂-Fc.

The specificity of the developed sensor described herein was further evaluated with varying concentrations of other sugar molecules, such as fructose and sucrose (FIG. 11D). As shown in chronoamperometry measurements in FIG. 11C, the current increased with the addition of 5 mM glucose, indicating the functional sensitivity of our developed sensor for glucose detection. The same sensor, when subjected to varying fructose concentrations, showed no changes in the observed current response, followed by an increase in current response to 5 mM glucose, suggesting the high specificity of the NH₂-Fc functionalized nanocomposite coated sensor for glucose sensing.

### Glucose Detection in Biological Sample Matrices

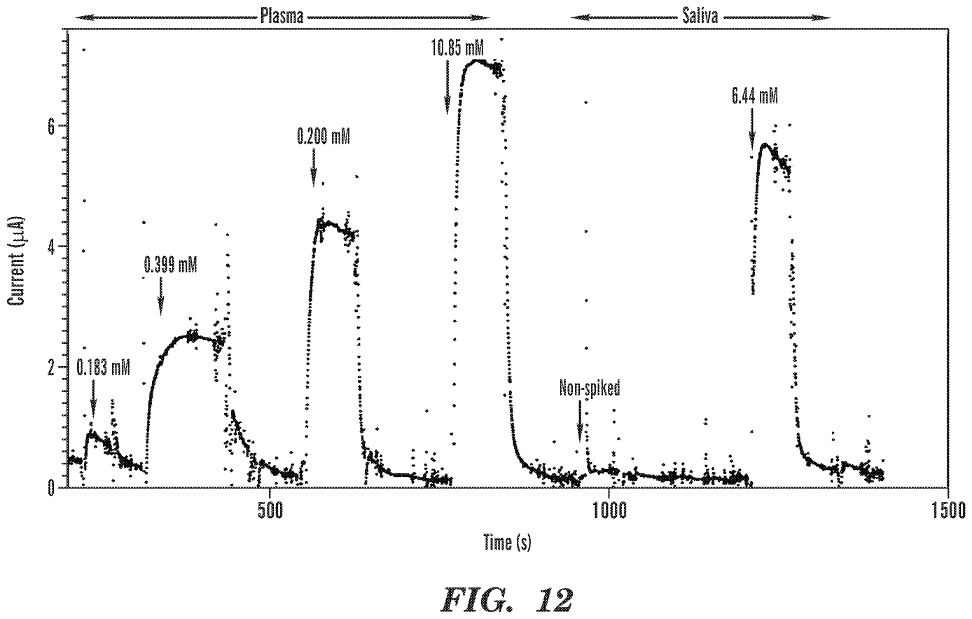

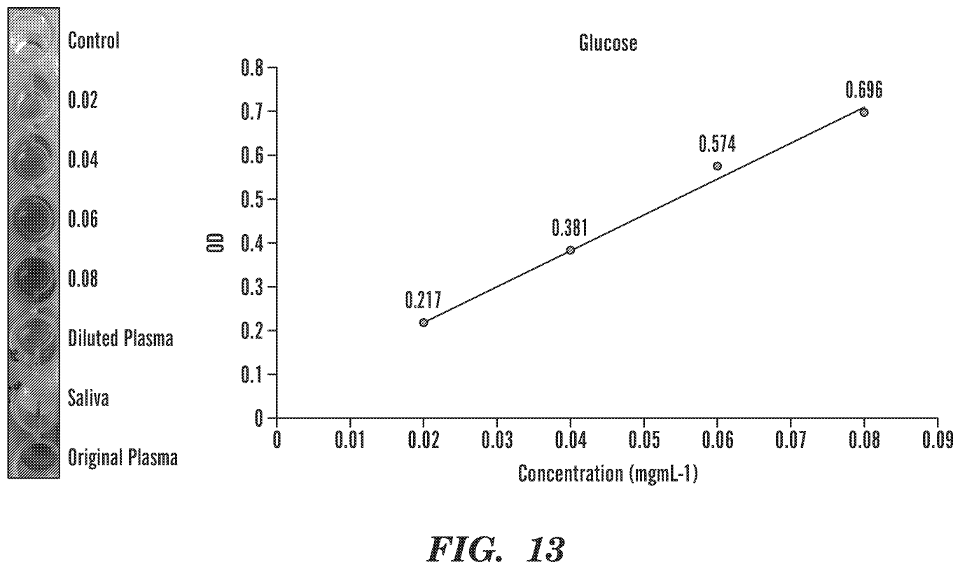

The feasibility of glucose detection in true biological samples is essential for determining clinical applications for this sensor. The performance of our NH₂-Fc functionalized glucose sensor was investigated using plasma and saliva samples containing different glucose concentrations (FIG. 12). The sensor consistently exhibited current responses in the detectable range, indicating the reliability and robustness of our developed sensor for clinical use. Sensor performance evaluation with real samples needs to be carried out further to evaluate the commercial viability of this glucose sensor.

### DISCUSSION

Enzyme-catalyzed glucose oxidation has been the standard for developing electrochemical-based glucose biosensors. The general chemical principle involves glucose oxidase, an enzyme that selectively oxidizes glucose to form gluconic acid and hydrogen peroxide (H₂O₂) in the presence of oxygen, as indicated by the following reaction:

$$Glucose + {H}_{2} O + {O}_{2} {\to }^{Glucose oxidase} Gluconic acid + {H}_{2} {O}_{2}$$

H₂O₂ is further oxidized at the working electrode, leading to the release of two free electrons, constituting an electrochemical signal response proportional to glucose concentration. However, detection approaches based on O₂ consumption or H₂O₂ production may not be suited for continuous monitoring due to oxygen limitations, especially for sensor systems employed in vivo. An alternative strategy is to replace O₂ with molecules that can serve as electron mediators, which forms the basis of our work.

Developing an enzymatic electrochemical readout based on a prGOx/BSA/GA nanocomposite-based antifouling coating with a functionalized redox molecule represents a significant advancement in metabolomics and diagnostic applications. This innovative approach addresses the challenges associated with cross-reactivity and signal instability in traditional diagnostic devices, enabling the continuous monitoring of metabolites, such as glucose, over a period of time. In this work, the charge transport between the redox center of glucose oxidase and nanocomposite-coated SPCE is achieved using NH₂-Fc based on the following reaction:

$$Glucose + {NH}_{2} - {Fc}^{+} {\to }^{Glucose oxidase} Gluconic acid + {NH}_{2} - Fc + 2 {H}^{+}$$

The electrochemical responses shown in FIG. 3 establish the role of NH₂-Fc, i.e., to tunnel electrons between the electrode and enzyme redox center, which is crucial in developing glucose biosensors for continuous monitoring. It was also established that the covalent coupling of NH₂-Fc to the nanocomposite-modified SPCE prevents the leaching of NH₂-Fc, which is required for building a sensitive detection system (FIG. 3D). The linear relationship between glucose concentration and current response demonstrates adequate glucose oxidase loading in the enzyme layer formed via GA crosslinking. Furthermore, the reproducible current response in the presence of various interfering species and biological sample matrix demonstrates the potential of developed biosensors for designing low-cost, rapid, sensitive, and portable electrochemical systems for monitoring multiple metabolites. The simplistic fabrication methods and the flexibility of modifying the enzyme layer would allow for quicker optimization of the developed biosensor platform to different target analytes, opening new avenues for continuous monitoring. In addition, multivariate analysis can be effectively achieved by leveraging advancements in machine learning, resulting in a more accurate and rapid biosensor.

### CONCLUSIONS

In summary, described herein are glucose biosensors that address critical challenges in glucose detection. Described herein is a highly sensitive and selective biosensor capable of quantifying metabolites, such as glucose, using the integrated assembly of a nanocomposite-based antifouling coating, a functionalized ferrocene-based redox mediator, and a redox enzyme. An Fc derivative of NH₂-Fc exhibits a reversible redox behavior, facilitating electron transport with the redox center of glucose oxidase and supporting the regeneration of enzyme activity for continuous determination of glucose. Clinical application of the developed platform is demonstrated in plasma and saliva samples and shows no interference from other electroactive species. The utilization of alternative biological matrices for detection expands the application scope of the biosensor for several other biological matrices. This versatility broadens its potential applications in clinical diagnostics, metabolic profiling, and drug development, contributing to advancements in personalized medicine and healthcare practices.

### REFERENCES

1. Ong, K. L.; Stafford, L. K.; Mclaughlin, S. A.; Boyko, E. J.; Vollset, S. E.; Smith, A. E.; Dalton, B. E.; Duprey, J.; Cruz, J. A.; Hagins, H.; et al. Global, regional, and national burden of diabetes from 1990 to 2021, with projections of prevalence to 2050: a systematic analysis for the Global Burden of Disease Study 2021. The Lancet 2023, 402 (10397), 203-234.
2. Leon, B. M.; Maddox, T. M. Diabetes and cardiovascular disease: Epidemiology, biological mechanisms, treatment recommendations and future research. World J Diabetes 2015, 6 (13), 1246-1258. DOI: 10.4239/wjd.v6.i13.1246.
3. Peng, Z.; Xie, X.; Tan, Q.; Kang, H.; Cui, J.; Zhang, X.; Li, W.; Feng, G. Blood glucose sensors and recent advances: A review. Journal of Innovative Optical Health Sciences 2022, 15 (02), 2230003. DOI: 10.1142/s1793545822300038.
4. CDC. National Diabetes Statistics Report: Estimates of Diabetes and Its Burden in the United States. 2023.
5. Noorhashimah Mohamad Nor, N. S. R., Khairunisak Abdul Razak. Generations of Glucose Biosensors. Encylopedia—Nanoscience & Nanotechnology 2022.
6. Pullano, S. A.; Greco, M.; Bianco, M. G.; Foti, D.; Brunetti, A.; Fiorillo, A. S. Glucose biosensors in clinical practice: principles, limits and perspectives of currently used devices. Theranostics 2022, 12 (2), 493-511. DOI: 10.7150/thno.64035.
7. Olejnik, A.; Karczewski, J.; Dołęga, A.; Siuzdak, K.; Grochowska, K. Novel approach to interference analysis of glucose sensing materials coated with Nafion. Bioelectrochemistry 2020, 135, 107575.
8. Witkowska Nery, E.; Kundys, M.; Jeleń, P. S.; Jönsson-Niedziółka, M. Electrochemical Glucose Sensing: Is There Still Room for Improvement? Analytical Chemistry 2016, 88 (23), 11271-11282.
9. Erbach, M.; Freckmann, G.; Hinzmann, R.; Kulzer, B.; Ziegler, R.; Heinemann, L.; Schnell, O. Interferences and Limitations in Blood Glucose Self-Testing: An Overview of the Current Knowledge. J Diabetes Sci Technol 2016, 10 (5), 1161-1168. DOI: 10.1177/1932296816641433.
10. Majewski, J.; Risler, Z.; Gupta, K. Erroneous Causes of Point-of-Care Glucose Readings. Cureus 2023, 15 (3), e36356. DOI: 10.7759/cureus.36356.
11. Klonoff, D. C. Continuous glucose monitoring: roadmap for 21st century diabetes therapy. Diabetes Care 2005, 28 (5), 1231-1239. DOI: 10.2337/diacare.28.5.1231 From NLM.
12. Vaddiraju, S.; Burgess, D. J.; Tomazos, I.; Jain, F. C.; Papadimitrakopoulos, F. Technologies for continuous glucose monitoring: current problems and future promises. J Diabetes Sci Technol 2010, 4 (6), 1540-1562. DOI: 10.1177/193229681000400632 From NLM.
13. Johnston, L.; Wang, G.; Hu, K.; Qian, C.; Liu, G. Advances in Biosensors for Continuous Glucose Monitoring Towards Wearables. Front Bioeng Biotechnol 2021, 9, 733810. DOI: 10.3389/fbioe.2021.733810 From NLM.
14. Janapala, R. N.; Jayaraj, J. S.; Fathima, N.; Kashif, T.; Usman, N.; Dasari, A.; Jahan, N.; Sachmechi, I. Continuous Glucose Monitoring Versus Self-monitoring of Blood Glucose in Type 2 Diabetes Mellitus: A Systematic Review with Meta-analysis. Cureus 2019, 11 (9), e5634. DOI: 10.7759/cureus.5634 From NLM.
15. Sharma, H.; Kalita, D.; Naskar, U.; Mishra, B. K.; Kumar, P.; Mirza, K. B. Prediction of Glucose Sensor Sensitivity in the Presence of Biofouling Using Machine Learning and Electrochemical Impedance Spectroscopy. IEEE Sensors Journal 2023, 23 (16), 18785-18797. DOI: 10.1109/JSEN.2023.3289619.
16. Sabaté del Río, J.; Henry, O. Y.; Jolly, P.; Ingber, D. E. An antifouling coating that enables affinity-based electrochemical biosensing in complex biological fluids. Nature nanotechnology 2019, 14 (12), 1143-1149.
17. Timilsina, S. S.; Durr, N.; Yafia, M.; Sallum, H.; Jolly, P.; Ingber, D. E. Ultrarapid Method for Coating Electrochemical Sensors with Antifouling Conductive Nanomaterials Enables Highly Sensitive Multiplexed Detection in Whole Blood. Advanced healthcare materials 2022, 11 (8), e2102244.
18. Zupančič, U.; Jolly, P.; Estrela, P.; Moschou, D.; Ingber, D. E. Graphene Enabled Low-Noise Surface Chemistry for Multiplexed Sepsis Biomarker Detection in Whole Blood. Advanced Functional Materials 2021, 31 (16), 2010638.
19. Timilsina, S. S.; Ramasamy, M.; Durr, N.; Ahmad, R.; Jolly, P.; Ingber, D. E. Biofabrication of multiplexed electrochemical immunosensors for simultaneous detection of clinical biomarkers in complex fluids. Advanced Healthcare Materials 2022, 2200589.
20. Timilsina, S. S.; Jolly, P.; Durr, N.; Yafia, M.; Ingber, D. E. Enabling multiplexed electrochemical detection of biomarkers with high sensitivity in complex biological samples. Accounts of Chemical Research 2021, 54 (18), 3529-3539.
21. Najjar, D.; Rainbow, J.; Sharma Timilsina, S.; Jolly, P.; de Puig, H.; Yafia, M.; Durr, N.; Sallum, H.; Alter, G.; Li, J. Z. A lab-on-a-chip for the concurrent electrochemical detection of SARS-COV-2 RNA and anti-SARS-COV-2 antibodies in saliva and plasma. Nature biomedical engineering 2022, 1-11.
22. Sharma, D.; Lim, Y.; Lee, Y.; Shin, H. Glucose sensor based on redox-cycling between selectively modified and unmodified combs of carbon interdigitated array nanoelectrodes. Analytica chimica acta 2015, 889, 194-202.
23. Dey, R. S.; Raj, C. R. Redox-Functionalized Graphene Oxide Architecture for the Development of Amperometric Biosensing Platform. ACS Applied Materials & Interfaces 2013, 5 (11), 4791-4798. DOI: 10.1021/am400280u.
24. Bartlett, P. N.; Bradford, V. Q.; Whitaker, R. G. Enzyme electrode studies of glucose oxidase modified with a redox mediator. Talanta 1991, 38 (1), 57-63.
25. Kang, D.; Zuo, X.; Yang, R.; Xia, F.; Plaxco, K. W.; White, R. J. Comparing the Properties of Electrochemical-Based DNA Sensors Employing Different Redox Tags. Analytical Chemistry 2009, 81 (21), 9109-9113. DOI: 10.1021/ac901811n.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. An electrode comprising: (i) a conductive substrate; and (ii) an antifouling coating layer on at least a portion of a surface of the conductive substrate, and wherein the antifouling coating layer comprises: a proteinaceous material, a conductive element, a redox mediator covalently linked to the proteinaceous material or the conductive element, and an oxidoreductase covalently linked to the proteinaceous material or the conductive element.

2. The electrode of claim 1, wherein the antifouling coating layer is adapted for contact with an analyte or a sample comprising an analyte.

3. The electrode of any one of claims 1-2, wherein the antifouling coating layer is porous.

4. The electrode of any one of claims 1-3, wherein the antifouling coating layer has a porosity of about 5% to about 95%.

5. The electrode of any one of claims 1-4, wherein the antifouling coating layer comprises macropores.

6. The electrode of any one of claims 1-5, wherein the antifouling coating layer comprises mesopores.

7. The electrode of any one of claims 1-6, wherein the antifouling coating layer comprises nanopores.

8. The electrode of any one of claims 1-7, wherein the redox mediator is covalently linked to the proteinaceous material or the conductive element by a cross-linker.

9. The electrode of claim 8, wherein the cross-linker is glutaraldehyde, genipin, polyethylene glycol, or a carbodiimide based cross-linker.

10. The electrode of any one of claims 1-9, wherein the redox mediator is covalently linked with the proteinaceous material.

11. The electrode of any one of claims 1-10, wherein the redox mediator is covalently linked with the conductive element.

12. The electrode of any one of claims 1-11, wherein the redox mediator is on a surface of the antifouling coating layer.

13. The electrode of any one of claims 1-12, wherein the redox mediator is embedded within the antifouling coating layer.

14. The electrode of any one of claims 1-13, wherein the redox mediator is selected from the group consisting of metallocenes metallocence derivatives, quinones, polycyclic aromatic hydrocarbons, redox-active organic molecules, phenazines, organosulfur compounds, and radical acceptors.

15. The electrode of any one of claims 1-14, wherein the redox mediator is selected from the group consisting of ferrocene, ferrocene derivatives, 3,3′,5,5′-tetramethylbenzidine (TMB), o-phenylenediamine dihydrochloride (OPD), N,N,N′,N′-tetramethyl-p-phenylenediamine, viologens 2,2′-azino-bis(3-ethylbenzothiazoline-6-sulfonic acid) (ABTS), p-nitrophenyl Phosphate (PNPP), 3,3′-diaminobenzidine (DAB), 4-chloro-1-naphthol (4-CN), 5-bromo-4-chloro-3-indolyl-phosphate (BCIP), nitro blue tetrazolium (NBT), methylene blue, tetrathialfulvaene, 2,6-dichloroindophenol (DCIP), 2,6-dichloroindophenyl phosphate, riboflavin 5′-monophosphate (RMP), ethyl viologen (1,1′-bis(ethyl)-4,4′-bipyridinium or BEBP), Paraquat, hexaamine ruthenium, 1,4-naphthoquione, resorufine, cyanomethylviologen, diquat, triquat, 1,1′-bis(2-sulfoethyl)-4,4′-bipyridinium, 1,1′-dibenzyl-4,4′-bipyridinium, 4,4′-dicarboxy-2,2′-bipyridyl, 1-hydroxybenzotriazole, veratryl alcohol, violuric acid, 2-methoxy-phenothiazone, 3-hydroxyanthranilic acid, anthraquinone 2,6-disulfonic acid, N-hydroxyacetanilide, phenol red, 3,3′,5,5′-tetramethyl benzidine, dichlorophenol red, 2,2′,6,6′-tetramethylpiperidine-N-oxyl radical, syringaldehyde and acetosyringone, 2,2,6,6-tetramethylpiperidin-1-yl)oxyl (TEMPO), 2,2′7,7′-tetrakis-(N,N-di-p-methoxyphenyl-amine)-9,9′-spirobifluorene (spiro-MeOTAD), sodium anthraquinone-2,6-di sulphonate (AQDS), benzoquinones, 2,2′-biimidazole, 2-(2-pyridyl) imidazole, 2,2′-bipyridine, tetracyanoquinodimethane (TCNQ), and combinations thereof.

16. The electrode of any one of claims 1-15, wherein the redox mediator is ferrocene or a derivative thereof.

17. The electrode of any one of claims 1-16, wherein the redox mediator is selected from the group consisting of aminoferrocene, ferrocene monocarboxylic acid, ferrocyanide, 1,1′-ferrocene dicarboxylic acid, 1,1′-dimethylferrocene (DMF), polyvinylferrocene, [N-ferrocenoyl]-4-aminophenyl phosphate, ferrocenylmethyl methacrylate, B-ferrocenyl-propenoic acid, and ferrocene monocarboxylic acid (FMCA).

18. The electrode of any one of claims 1-17, wherein the redox mediator is aminoferrocene.

19. The electrode of any one of claims 1-18, wherein the redox mediator is present in an amount from about 0.001 to about 10 (w/w).

20. The electrode of any one of claims 1-19, wherein a ratio of the redox mediator to the oxidoreductase is from about 1:100 to about 1:1 (w/w).

21. The electrode of any one of claims 1-20, wherein a ratio of the redox mediator to the proteinaceous material is from about 1:10 to about 1:1000 (w/w).

22. The electrode of any one of claims 1-21, wherein a ratio of the redox mediator to the conductive element is from about 1:10 to about 1:1000 (w/w).

23. The electrode of any one of claims 1-22, wherein the oxidoreductase is covalently linked to the proteinaceous material or the conductive element by a cross-linker.

24. The electrode of claim 23, wherein the cross-linker is glutaraldehyde, genipin, or polyethylene glycol.

25. The electrode of any one of claims 1-24, wherein the oxidoreductase is covalently linked with the proteinaceous material.

26. The electrode of any one of claims 1-25, wherein the oxidoreductase is covalently linked with the conductive element.

27. The electrode of any one of claims 1-26, wherein the oxidoreductase is on a surface of the antifouling coating layer.

28. The electrode of any one of claims 1-27, wherein the oxidoreductase is embedded within the antifouling coating layer.

29. The electrode of any one of claims 1-28, wherein the oxidoreductase is selected from the group consisting of oxidases, reductases, dehydrogenases, peroxidases, oxygenases, monooxygenases, dioxygenases, lipoxygenases, hydrogenases, transhydrogenases, peroxidases, catalases, epoxidases, hydroxylases, demethylases, desaturases, dismutases, hydroxyltransferases, synthases, dehalogenases, and deiodinases.

30. The electrode of any one of claims 1-29, wherein the oxidoreductase is selected from the group consisting of glucose oxidase, malate oxidase, hexose oxidase, aryl-alcohol oxidase, alcohol oxidase, long-chain alcohol oxidase, glycerol-3-phosphate oxidase, poly vinyl-alcohol oxidase, D-arabinono-1,4-lactone oxidase, D-mannitol oxidase, xylitol oxidase, oxalate oxidase, carbon-monoxide oxidase, 4-hydroxyphenylpyruvate oxidase, dihydrouracil oxidase, ethanolamine oxidase, lactate oxidase, L-aspartate oxidase, sarcosine oxidase, urate oxidase, methanethiol oxidase, 3-hydroxyanthranilate oxidase, cholesterol oxidase, xanthine oxidase, amino-acid oxidase, laccase, catalase, fatty-acid peroxidase, peroxidase, diarylpropane peroxidase, ferroxidase, pteridine oxidase, columbamine oxidase, catechol 1,2-dioxygenase, gentisate 1,2-dioxygenase, homogentisate 1,2-dioxygenase, lipoxygenase, ascorbate 2,3-dioxygenase, 3-carboxyethylcatechol 2,3-dioxygenase, indole 2,3-dioxygenase, caffeate 3,4-dioxygenase, arachidonate 5-lipoxygenase, biphenyl-2,3-diol 1,2-dioxygenase, linoleate 11-lipoxygenase, acetylacetone-cleaving enzyme, lactate 2-monooxygenase, phenylalanine 2-monooxygenase, inositol oxygenase, fructose dehydrogenase, alcohol dehydrogenase, glycerol dehydrogenase, propanediol-phosphate dehydrogenase, L-lactate dehydrogenase, D-lactate dehydrogenase, glycerate dehydrogenase, glucose 1-dehydrogenase, galactose 1-dehydrogenase, allyl-alcohol dehydrogenase, 4-hydroxybutyrate dehydrogenase, octanol dehydrogenase, aryl-alcohol dehydrogenase, cyclopentanol dehydrogenase, long-chain-3-hydroxyacyl-CoA dehydrogenase, butanal dehydrogenase, terephthalate 1,2-cis-dihydrodiol dehydrogenase, succinate dehydrogenase, glutamate dehydrogenase, glycine dehydrogenase, hydrogen dehydrogenase, 4-cresol dehydrogenase, phosphonate dehydrogenase, diethyl 2-methyl-3-oxosuccinate reductase, tropinone reductase, long-chain-fatty-acyl-CoA reductase, carboxylate reductase, D-proline reductase, glycine reductlactase, Heme-proteins such as cytochromes, carbon-carbon lyases, carbon-oxygen lyases, carbon-nitrogen lyases, carbon-sulfur lyases, carbon-halide lyases, and phosphorus-oxygen lyases, alkaline phosphatase, malate dehydrogenase, staphylococcal nuclease, delta-V-steroid isomerase, yeast alcohol dehydrogenase, alpha-glycerophosphate dehydrogenase, triose phosphate isomerase, horseradish peroxidase (HRP), asparaginase, beta-galactosidase, ribonuclease, urease, catalase, glucose-VI-phosphate dehydrogenase, glucoamylase, tyrosinase, pyruvate oxidase, aldehyde oxidase, carbon-monoxide oxidase, retinal oxidase, abscisic-aldehyde oxidase, (methyl) glyoxal oxidase, xanthine oxidase, oxalate oxidase, and acetylcholinesterase.

31. The electrode of any one of claims 1-30, wherein the oxidoreductase is glucose oxidase.

32. The electrode of any one of claims 1-31, wherein the oxidoreductase is present in an amount from about 20% to about 80% (w/w).

33. The electrode of any one of claims 1-32, wherein a ratio of the oxidoreductase to the proteinaceous material is about 10:1 to about 5:1 (w/w).

34. The electrode of any one of claims 1-33, wherein a ratio of the oxidoreductase to the conductive element is about 10:1 to about 5:1 (w/w).

35. The electrode of any one of claims 1-34, wherein the conducting material comprises conductive particles, conductive rods, conductive fibers, conductive nano-particles, conductive polymers, conductive nano-flakes, conductive nanotubes, semi-conductive particles, semi-conductive rods, semi-conductive fibers, semi-conductive nano-particles, semi-conductive nano-flakes, semi-conductive nanotubes, or semi-conductive polymers.

36. The electrode of any one of claims 1-35, wherein the conducting material is a metal, a metalloid, conducting polymer, a conducting carbon-based material, organic compounds having conducting and/or semiconducting properties, or any combination thereof.

37. The electrode of any one of claims 1-36, wherein the conducting material comprises an allotrope of carbon atoms arranged in a hexagonal lattice.

38. The electrode of any one of claims 1-37, wherein the conducting material is graphite, functionalised graphene oxide, reduced graphene oxide, or carbon nano-tubes (CNTs).

39. The electrode of claim 38, wherein the carbon nanotubes are carboxylated carbon nanotubes or aminated carbon nanotubes.

40. The electrode of claim 38, wherein the reduced graphene oxide is a carboxylated reduced graphene oxide or an aminated reduced graphene oxide.

41. The electrode of any one of claims 1-36, wherein the conductive material comprises gold.

42. The electrode of any one of claims 1-36, wherein the conductive material comprises one or more organic compounds having conducting and/or semiconducting properties.

43. The electrode of claim 42, wherein the organic compounds having conducting and/or semiconducting properties are selected from the group consisting of polyacetylenes, porphyrins, phthalocyanins, fullerenes, polyparaphenylenes, polyphenylenevinylenes, polyfluorenes, polythiophenes, polypyrroles, polypyridines, polycarbazoles, polypyridinevinylenes, polyarylvinylenes, poly(p-phenylmethylvinylenes), derivatives and co-polymers thereof, and any combination thereof.

44. The electrode of claim 42 or 43, wherein the organic compounds having conducting and/or semiconducting properties are selected from the group consisting of poly(3-hexylthiophene) (P3HT), perfluoro-3,6-dioxa-4-methyl-7-octenesulfonic acid copolymer, tetrafluoroethylene-perfluoro-3,6-dioxa-4-methyl-7-octenesulfonic acid copolymer, poly(9,9-dioctylfluorene-2,7-diyl-co-bis-N,N-(4-butylphenyl)-bis-N,N-phenyl-1,4-phenylenediamine), poly(9,9-dioctylfluorene-2,7-diyl-co-benzothiadiazole), (6,6)-phenyl-C61-butyric acid methyl ester, poly(2-methoxy-5-(2′-ethyl-hexyloxy)-1,4-phenylene vinylene), poly(4-vinylphenol) (PVP), and copolymers thereof, and any combination thereof.

45. The electrode of any one of claims 1-44, wherein the conductive element is present in an amount from about 0.01% to about 10% (w/v).

46. The electrode of any one of claims 1-45, wherein a ratio of the proteinaceous material to the conductive element is from about 10:1 to about 1:1 (w/w).

47. The electrode of any one of claims 1-46, wherein the proteinaceous material is denatured.

48. The electrode of any one of claims 1-47, wherein the proteinaceous material is non-reversibly denatured.

49. The electrode of any one of claims 1-48, wherein the proteinaceous material is a globular protein.

50. The electrode of any one of claims 1-49, wherein the proteinaceous material is a non-glycosylated protein.

51. The electrode of any one of claims 1-50, wherein the proteinaceous material is a serum albumin protein.

52. The electrode of any one of claims 1-51, wherein the proteinaceous material is bovine serum albumin (BSA) or human serum albumin (HSA).

53. The electrode of any one of claims 1-52, wherein the proteinaceous material is cross-linked with the conductive element.

54. The electrode of any one of claims 1-53, wherein the proteinaceous material is cross-linked to itself.

55. The electrode of any one of claims 1-54, wherein the proteinaceous material is covalently linked to the conductive surface.

56. The electrode of any one of claims 1-55, wherein the proteinaceous material is cross-linked by a cross-linker.

57. The electrode of claim 53, wherein the cross-linker is glutaraldehyde, genipin, or polyethylene glycol.

58. The electrode of any one of claims 1-57, wherein the antifouling coating layer further comprises a target binding molecule capable of binding with a target molecule.

59. The electrode of claim 58, wherein the target binding molecule is covalently linked to the proteinaceous material, or the conductive or semi-conductive material.

60. The electrode of claim 58 or 59, wherein the target binding molecule is a receptor, a ligand for a receptor, an antibody, antigen binding fragment of an antibody, an antigen, an enzyme or a nucleic acid.

61. The electrode of any one of claims 58-60, wherein the target binding molecule is an antibody, an antigen binding fragment of an antibody or an antigen.

62. The electrode of any one of claims 58-61, wherein the antifouling coating layer comprises an anti-microbial agent.

63. The electrode of claim 62, wherein the anti-microbial agent is an anti-bacterial agent, anti-fungal agent or anti-viral agent.

64. The electrode of claim 62 or 63, wherein the anti-microbial agent is an anti-bacterial agent.

65. The electrode of claim 64, wherein the anti-bacterial agent is selected from the group consisting of macrolides or ketolides such as erythromycin, azithromycin, clarithromycin, and telithromycin; beta-lactams including penicillin, cephalosporin, and carbapenems such as carbapenem, imipenem, and meropenem; monolactams such as penicillin G, penicillin V, methicillin, oxacillin, cloxacillin, dicloxacillin, nafcillin, ampicillin, amoxicillin, carbenicillin, ticarcillin, meziocillin, piperacillin, azlocillin, temocillin, cepalothin, cephapirin, cephradine, cephaloridine, cefazolin, cefamandole, cefuroxime, cephalexin, cefprozil, cefaclor, loracarbef, cefoxitin, cefmetazole, cefotaxime, ceftizoxime, cefiriaxone, cefoperazone, ceftazidime, cefixime, cefpodoxime, ceftibuten, cefdinir, cefpirome, cefepime, cefadroxil, ceftriaxone, ceftobiprole and astreonam; quinolones such as nalidixic acid, oxolinic acid, norfloxacin, pefloxacin, enoxacin, ofloxacin, levofloxacin, ciprofloxacin, temafloxacin, lomefloxacin, fleroxacin, grepafloxacin, sparfloxacin, trovafloxacin, clinafloxacin, gatifloxacin, moxifloxacin, sitafloxacin, ganefloxacin, gemifloxacin and pazufloxacin; antibacterial sulfonamides and antibacterial sulphanilamides, including para-aminobenzoic acid, sulfadiazine, sulfisoxazole, sulfamethoxazole and sulfathalidine; aminoglycosides such as streptomycin, neomvcin, kanamycin, paromycin, gentamicin, tobramycin, amikacin, netilmicin, spectinomycin, sisomicin, dibekalin and isepamicin; tetracyclines such as tetracycline, chlortetracycline, demeclocycline, minocycline, oxytetracycline, methacycline, doxycycline; rifamycins such as rifampicin (also called rifampin), rifapentine, rifabutin, bezoxazinorifamycin and rifaximin; lincosamides such as lincomycin and clindamycin; glycopeptides such as vancomycin and teicoplanin; streptogramins such as quinupristin and daflopristin; oxazolidinones such as linezolid; polymyxin, colistin and colymycin; trimethoprim, bacitracin, and phosphonomycin.

66. The electrode of claim 62 or 63, wherein the anti-microbial agent is an antifungal agent.

67. The electrode of claim 66, wherein the antifungal agent is selected from the group consisting of azoles, polyenes, echinocandins, pradimicins, Triclosan, Piroctone, fenpropimorph, terbinafine, cyclopyroxolamine, flucitocin, griseofulvin haloprozin, tolnaftate, naphthypine, hydrochloride, morpholine, butenapin, undecylenic acid, propionic acid, and derivatives and analogs thereof.

68. The electrode of claim 62 or 63, wherein the anti-microbial agent is an antimicrobial peptide or polymer.

69. The electrode of claim 62 or 63, wherein the anti-microbial agent is a metal particle.

70. The electrode of claim 69, wherein anti-microbial agent is titanium oxide, copper, or silver nanoparticles.

71. The electrode of any one of claims 1-70, wherein the antifouling coating layer further comprises a therapeutic agent.

72. The electrode of any one of claims 1-71, wherein the antifouling coating layer further comprises a polymer.

73. The electrode of claim 72, wherein the polymer is a water miscible polymer.

74. The electrode of claim 72 or 73, wherein the polymer is a degradable polymer

75. The electrode of any one of claims 72-74, wherein the polymer is selected from the group consisting of poly(N-isopropyl acrylamide) (PNIPAAm), polyethylene glycol (PEG), alginate, polytetrafluoroethylene (PTFE), polyacetylene (PA), polyaniline (PANI), polypyrrole (PPy), polythiophene (PTH), poly(para-phenylene) (PPP), poly(phenylenevinylene) (PPV), and polyfuran (PF).

76. The electrode of any one of claims 1-75, wherein the electrode is a planar or 3-dimensional electrode.

77. The electrode of any one of claims 1-76, wherein the conductive substrate comprises gold, silver, copper, platinum, aluminum, stainless steel, tungsten, indium tin oxide, titanium, lead, nickel, palladium, zirconium, niobium, tantalum, chromium, molybdenum, manganese, rhenium, ruthenium, rhodium, iridium, silicon, polyimide, parylene, benzocyclobutene, carbon, graphite, or any combination thereof.

78. The electrode of any one of claims 1-77, wherein the conductive substrate comprises a flexible substrate.

79. The electrode of claim 78, wherein the flexible substrate comprises polyethylene terephthalate, polyethylene naphathalate, polyimides, polymeric hydrocarbons, celluloses, plastics, polycarbonates, polystyrenes, or any combination thereof.

80. The electrode of any one of claims 1-79, wherein the coating layer has a thickness from about 2 nm to about 100 μm.

81. A sensor comprising an electrode of any one of claims 1-80.

82. The sensor of claim 81, wherein the sensor comprises a fluid-contact surface and the electrode is immobilized on at least a portion of the fluid-contact surface.

83. The sensor of any one of claims 81-82, wherein the sensor comprises one or more microfluidic flow cells.

84. The sensor of any one of claims 81-83, wherein the fluid-contact surface further comprises a positive control electrode and/or a negative control electrode immobilized thereon.

85. A composition comprising: a proteinaceous material, a conductive element, a redox mediator covalently linked to the proteinaceous material or the conductive element, and an oxidoreductase covalently linked to the proteinaceous material or the conductive element.

86. The composition of claim 85, wherein the redox mediator is covalently linked to the proteinaceous material or the conductive element by a cross-linker.

87. The composition of claim 86, wherein the cross-linker is glutaraldehyde, genipin, or polyethylene glycol.

88. The composition of any one of claims 85-87, wherein the redox mediator is covalently linked with the proteinaceous material.

89. The composition of any one of claims 85-88, wherein the redox mediator is covalently linked with the conductive element.

90. The composition of any one of claims 85-89, wherein the redox mediator is selected from the group consisting of metallocenes metallocence derivatives, quinones, polycyclic aromatic hydrocarbons, redox-active organic molecules, phenazines, organosulfur compounds, and radical.

91. The composition of any one of claims 85-90, wherein the redox mediator is selected from the group consisting of ferrocene, ferrocene derivatives, 3,3′,5,5′-tetramethylbenzidine (TMB), o-phenylenediamine dihydrochloride (OPD), N,N,N′,N′-tetramethyl-p-phenylenediamine, viologens 2,2′-azino-bis(3-ethylbenzothiazoline-6-sulfonic acid) (ABTS), p-nitrophenyl Phosphate (PNPP), 3,3′-diaminobenzidine (DAB), 4-chloro-1-naphthol (4-CN), 5-bromo-4-chloro-3-indolyl-phosphate (BCIP), nitro blue tetrazolium (NBT), methylene blue, tetrathialfulvaene, 2,6-dichloroindophenol (DCIP), 2,6-dichloroindophenyl phosphate, riboflavin 5′-monophosphate (RMP), ethyl viologen (1,1′-bis(ethyl)-4,4′-bipyridinium or BEBP), Paraquat, hexaamine ruthenium, 1,4-naphthoquione, resorufine, cyanomethylviologen, diquat, triquat, 1,1′-bis(2-sulfoethyl)-4,4′-bipyridinium, 1,1′-dibenzyl-4,4′-bipyridinium, 4,4′-dicarboxy-2,2′-bipyridyl, 1-hydroxybenzotriazole, veratryl alcohol, violuric acid, 2-methoxy-phenothiazone, 3-hydroxyanthranilic acid, anthraquinone 2,6-disulfonic acid, N-hydroxyacetanilide, phenol red, 3,3′,5,5′-tetramethyl benzidine, dichlorophenol red, 2,2′,6,6′-tetramethylpiperidine-N-oxyl radical, syringaldehyde and acetosyringone, 2,2,6,6-tetramethylpiperidin-1-yl)oxyl (TEMPO), 2,2′7,7′-tetrakis-(N,N-di-p-methoxyphenyl-amine)-9,9′-spirobifluorene (spiro-MeOTAD), sodium anthraquinone-2,6-di sulphonate (AQDS), benzoquinones, 2,2′-biimidazole, 2-(2-pyridyl) imidazole, 2,2′-bipyridine, tetracyanoquinodimethane (TCNQ), and combinations thereof.

92. The composition of any one of claims 85-91, wherein the redox mediator is ferrocene or a derivative thereof.

93. The composition of any one of claims 85-92, wherein the redox mediator is selected from the group consisting of aminoferrocene, ferrocene monocarboxylic acid, ferrocyanide, 1,1′-ferrocene dicarboxylic acid, 1,1′-dimethylferrocene (DMF), polyvinylferrocene, [N-ferrocenoyl]-4-aminophenyl phosphate, ferrocenylmethyl methacrylate, and ferrocene monocarboxylic acid (FMCA).

94. The composition of any one of claims 85-93, wherein the redox mediator is aminoferrocene.

95. The composition of any one of claims 85-94, wherein the redox mediator is present in an amount from about 0.001 to about 10 (w/w).

96. The composition of any one of claims 85-95, wherein a ratio of the redox mediator to the oxidoreductase is from about 1:100 to about 1:1 (w/w).

97. The composition of any one of claims 85-96, wherein a ratio of the redox mediator to the proteinaceous material is from about 1:10 to about 1:1000 (w/w). The composition of any one of the preceding claims, wherein a ratio of the redox mediator to the conductive element is from about 1:10 to about 1:1000 (w/w).

98. The composition of any one of claims 85-97, wherein the oxidoreductase is covalently linked to the proteinaceous material or the conductive element by a cross-linker.

99. The composition of claim 98, wherein the cross-linker is glutaraldehyde, genipin, or polyethylene glycol.

100. The composition of any one of claims 85-99, wherein the oxidoreductase is covalently linked with the proteinaceous material.

101. The composition of any one of claims 85-100, wherein the oxidoreductase is covalently linked with the conductive element.

102. The composition of any one of claims 85-101, wherein the oxidoreductase is selected from the group consisting of oxidases, reductases, dehydrogenases, peroxidases, oxygenases, monooxygenases, dioxygenases, lipoxygenases, hydrogenases, transhydrogenases, peroxidases, catalases, epoxidases, hydroxylases, demethylases, desaturases, dismutases, hydroxyltransferases, synthases, dehalogenases, and deiodinases.

103. The composition of any one of claims 85-102, wherein the oxidoreductase is selected from the group consisting of glucose oxidase, malate oxidase, hexose oxidase, aryl-alcohol oxidase, alcohol oxidase, long-chain alcohol oxidase, glycerol-3-phosphate oxidase, poly vinyl-alcohol oxidase, D-arabinono-1,4-lactone oxidase, D-mannitol oxidase, xylitol oxidase, oxalate oxidase, carbon-monoxide oxidase, 4-hydroxyphenylpyruvate oxidase, dihydrouracil oxidase, ethanolamine oxidase, lactate oxidase, L-aspartate oxidase, sarcosine oxidase, urate oxidase, methanethiol oxidase, 3-hydroxyanthranilate oxidase, cholesterol oxidase, xanthine oxidase, amino-acid oxidase, laccase, catalase, fatty-acid peroxidase, peroxidase, diarylpropane peroxidase, ferroxidase, pteridine oxidase, columbamine oxidase, catechol 1,2-dioxygenase, gentisate 1,2-dioxygenase, homogentisate 1,2-dioxygenase, lipoxygenase, ascorbate 2,3-dioxygenase, 3-carboxyethylcatechol 2,3-dioxygenase, indole 2,3-dioxygenase, caffeate 3,4-dioxygenase, arachidonate 5-lipoxygenase, biphenyl-2,3-diol 1,2-dioxygenase, linoleate 11-lipoxygenase, acetylacetone-cleaving enzyme, lactate 2-monooxygenase, phenylalanine 2-monooxygenase, inositol oxygenase, fructose dehydrogenase, alcohol dehydrogenase, glycerol dehydrogenase, propanediol-phosphate dehydrogenase, L-lactate dehydrogenase, D-lactate dehydrogenase, glycerate dehydrogenase, glucose 1-dehydrogenase, galactose 1-dehydrogenase, allyl-alcohol dehydrogenase, 4-hydroxybutyrate dehydrogenase, octanol dehydrogenase, aryl-alcohol dehydrogenase, cyclopentanol dehydrogenase, long-chain-3-hydroxyacyl-CoA dehydrogenase, butanal dehydrogenase, terephthalate 1,2-cis-dihydrodiol dehydrogenase, succinate dehydrogenase, glutamate dehydrogenase, glycine dehydrogenase, hydrogen dehydrogenase, 4-cresol dehydrogenase, phosphonate dehydrogenase, diethyl 2-methyl-3-oxosuccinate reductase, tropinone reductase, long-chain-fatty-acyl-CoA reductase, carboxylate reductase, D-proline reductase, glycine reductlactase, Heme-proteins such as cytochromes, carbon-carbon lyases, carbon-oxygen lyases, carbon-nitrogen lyases, carbon-sulfur lyases, carbon-halide lyases, and phosphorus-oxygen lyases, alkaline phosphatase, malate dehydrogenase, staphylococcal nuclease, delta-V-steroid isomerase, yeast alcohol dehydrogenase, alpha-glycerophosphate dehydrogenase, triose phosphate isomerase, horseradish peroxidase (HRP), asparaginase, beta-galactosidase, ribonuclease, urease, catalase, glucose-VI-phosphate dehydrogenase, glucoamylase, tyrosinase, pyruvate oxidase, aldehyde oxidase, carbon-monoxide oxidase, retinal oxidase, abscisic-aldehyde oxidase, (methyl) glyoxal oxidase, xanthine oxidase, oxalate oxidase, and acetylcholinesterase.

104. The composition of any one of claims 85-103, wherein the oxidoreductase is glucose oxidase.

105. The composition of any one of claims 85-104, wherein the oxidoreductase is present in an amount from about 20% to about 80% (w/w).

106. The composition of any one of claims 85-105, wherein a ratio of the oxidoreductase to the proteinaceous material is about 10:1 to about 5:1 (w/w).

107. The composition of any one of claims 85-106, wherein a ratio of the oxidoreductase to the conductive element is about 10:1 to about 5:1 (w/w).

108. The composition of any one of claims 85-107, wherein the conducting material comprises conductive particles, conductive rods, conductive fibers, conductive nano-particles, conductive polymers, conductive nano-flakes, conductive nanotubes, semi-conductive particles, semi-conductive rods, semi-conductive fibers, semi-conductive nano-particles, semi-conductive nano-flakes, semi-conductive nanotubes, or semi-conductive polymers.

109. The composition of any one of claims 85-108, wherein the conducting material is a metal, a metalloid, conducting polymer, a conducting carbon-based material, organic compounds having conducting and/or semiconducting properties, or any combination thereof.

110. The composition of any one of claims 85-109, wherein the conducting material comprises an allotrope of carbon atoms arranged in a hexagonal lattice.

111. The composition of any one of claims 85-110, wherein the conducting material is graphite, functionalised graphene oxide, reduced graphene oxide, or carbon nano-tubes (CNTs).

112. The composition of claim 111, wherein the carbon nanotubes are carboxylated carbon nanotubes or aminated carbon nanotubes.

113. The composition of claim 112, wherein the reduced graphene oxide is a carboxylated reduced graphene oxide or an aminated reduced graphene oxide.

114. The composition of any one of claims 85-109, wherein the conductive material comprises gold.

115. The composition of any one of claims 85-109, wherein the conductive material comprises one or more organic compounds having conducting and/or semiconducting properties.

116. The composition of claim 115, wherein the organic compounds having conducting and/or semiconducting properties are selected from the group consisting of polyacetylenes, porphyrins, phthalocyanins, fullerenes, polyparaphenylenes, polyphenylenevinylenes, polyfluorenes, polythiophenes, polypyrroles, polypyridines, polycarbazoles, polypyridinevinylenes, polyarylvinylenes, poly(p-phenylmethylvinylenes), derivatives and co-polymers thereof, and any combination thereof.

117. The composition of claim 115 or 116, wherein the organic compounds having conducting and/or semiconducting properties are selected from the group consisting of poly(3-hexylthiophene) (P3HT), perfluoro-3,6-dioxa-4-methyl-7-octenesulfonic acid copolymer, tetrafluoroethylene-perfluoro-3,6-dioxa-4-methyl-7-octenesulfonic acid copolymer, poly(9,9-dioctylfluorene-2,7-diyl-co-bis-N,N-(4-butylphenyl)-bis-N,N-phenyl-1,4-phenylenediamine), poly(9,9-dioctylfluorene-2,7-diyl-co-benzothiadiazole), (6,6)-phenyl-C61-butyric acid methyl ester, poly(2-methoxy-5-(2′-ethyl-hexyloxy)-1,4-phenylene vinylene), poly(4-vinylphenol) (PVP), and copolymers thereof, and any combination thereof.

118. The composition of any one of claims 85-117, wherein the conductive element is present in an amount from about 0.01% to about 10% (w/w).

119. The composition of any one of claims 85-118, wherein a ratio of the proteinaceous material to the conductive element is from about 10:1 to about 1:1 (w/w).

120. The composition of any one of claims 85-119, wherein the proteinaceous material is denatured.

121. The composition of any one of claims 85-120, wherein the proteinaceous material is non-reversibly denatured.

122. The composition of any one of claims 85-121, wherein the proteinaceous material is a globular protein.

123. The composition of any one of claims 85-122, wherein the proteinaceous material is a non-glycosylated protein.

124. The composition of any one of claims 85-123, wherein the proteinaceous material is a serum albumin protein.

125. The composition of any one of claims 85-124, wherein the proteinaceous material is bovine serum albumin (BSA) or human serum albumin (HSA).

126. The composition of any one of claims 85-125, wherein the proteinaceous material is cross-linked with the conductive element.

127. The composition of any one of claims 85-126, wherein the proteinaceous material is cross-linked to itself.

128. The composition of any one of claims 85-127, wherein the proteinaceous material is cross-linked by a cross-linker.

129. The composition of claim 128, wherein the cross-linker is glutaraldehyde, genipin, or polyethylene glycol.

130. The composition of any one of claims 85-129, wherein the composition further comprises a target binding molecule capable of binding with a target molecule.

131. The electrode of claim 130, wherein the target binding molecule is covalently linked to the proteinaceous material, or the conductive or semi-conductive material.

132. The electrode of claim 130 or 131, wherein the target binding molecule is a receptor, a ligand for a receptor, an antibody, antigen binding fragment of an antibody, an antigen, an enzyme or a nucleic acid.

133. The electrode of any one of claims 130-132, wherein the target binding molecule is an antibody, an antigen binding fragment of an antibody or an antigen.

134. The composition of any one of claims 85-133, wherein the composition comprises an anti-microbial agent.

135. The composition of claim 130, wherein the anti-microbial agent is an anti-bacterial agent, anti-fungal agent or anti-viral agent.

136. The composition of claim 130 or 131, wherein the anti-microbial agent is an anti-bacterial agent.

137. The composition of claim 128, wherein the anti-bacterial agent is selected from the group consisting of macrolides or ketolides such as erythromycin, azithromycin, clarithromycin, and telithromycin; beta-lactams including penicillin, cephalosporin, and carbapenems such as carbapenem, imipenem, and meropenem; monolactams such as penicillin G, penicillin V, methicillin, oxacillin, cloxacillin, dicloxacillin, nafcillin, ampicillin, amoxicillin, carbenicillin, ticarcillin, meziocillin, piperacillin, azlocillin, temocillin, cepalothin, cephapirin, cephradine, cephaloridine, cefazolin, cefamandole, cefuroxime, cephalexin, cefprozil, cefaclor, loracarbef, cefoxitin, cefmetazole, cefotaxime, ceftizoxime, cefiriaxone, cefoperazone, ceftazidime, cefixime, cefpodoxime, ceftibuten, cefdinir, cefpirome, cefepime, cefadroxil, ceftriaxone, ceftobiprole and astreonam; quinolones such as nalidixic acid, oxolinic acid, norfloxacin, pefloxacin, enoxacin, ofloxacin, levofloxacin, ciprofloxacin, temafloxacin, lomefloxacin, fleroxacin, grepafloxacin, sparfloxacin, trovafloxacin, clinafloxacin, gatifloxacin, moxifloxacin, sitafloxacin, ganefloxacin, gemifloxacin and pazufloxacin; antibacterial sulfonamides and antibacterial sulphanilamides, including para-aminobenzoic acid, sulfadiazine, sulfisoxazole, sulfamethoxazole and sulfathalidine; aminoglycosides such as streptomycin, neomvcin, kanamycin, paromycin, gentamicin, tobramycin, amikacin, netilmicin, spectinomycin, sisomicin, dibekalin and isepamicin; tetracyclines such as tetracycline, chlortetracycline, demeclocycline, minocycline, oxytetracycline, methacycline, doxycycline; rifamycins such as rifampicin (also called rifampin), rifapentine, rifabutin, bezoxazinorifamycin and rifaximin; lincosamides such as lincomycin and clindamycin; glycopeptides such as vancomycin and teicoplanin; streptogramins such as quinupristin and daflopristin; oxazolidinones such as linezolid; polymyxin, colistin and colymycin; trimethoprim, bacitracin, and phosphonomycin.

138. The composition of claim 130 or 131, wherein the anti-microbial agent is an antifungal agent.

139. The composition of claim 138, wherein the antifungal agent is selected from the group consisting of azoles, polyenes, echinocandins, Triclosan, Piroctone, fenpropimorph, terbinafine, cyclopyroxolamine, flucitocin, griseofulvin haloprozin, tolnaftate, naphthypine, hydrochloride, morpholine, butenapin, undecylenic acid, propionic acid, and derivatives and analogs thereof.

140. The composition of claim 130 or 131, wherein the anti-microbial agent is an antimicrobial peptide or polymer.

141. The composition of claim 130 or 131, wherein the anti-microbial agent is a metal particle.

142. The composition of claim 141, wherein anti-microbial agent is titanium oxide, copper, or silver nanoparticles.

143. The composition of any one of claims 85-142, wherein the composition further comprises a therapeutic agent.

144. The composition of any one of claims 85-143, wherein the composition further comprises a polymer.

145. The composition of claim 144, wherein the polymer is a water miscible polymer.

146. The composition of claim 144 or 145, wherein the polymer is a degradable polymer

147. The composition of any one of claims 144-146, wherein the polymer is selected from the group consisting of poly(N-isopropyl acrylamide) (PNIPAAm), polyethylene glycol, alginate, polytetrafluoroethylene (PTFE), polyacetylene (PA), polyaniline (PANI), polypyrrole (PPy), polythiophene (PTH), poly(para-phenylene) (PPP), poly(phenylenevinylene) (PPV), and polyfuran (PF).

148. A surface comprising an antifouling coating layer on at least a part of the surface, wherein the coating layer comprises a composition of any one of claims 85-147.

149. The surface of claim 148, wherein the coating layer is directly or indirectly connected with an electrode.

150. The surface of any one of claims 148-149, wherein the coating layer is adapted for contact with an analyte or a sample comprising an analyte.

151. The surface of any one of claims 148-150, wherein the coating layer is porous.

152. The surface of any one of claims 148-151, wherein the coating layer has a porosity of about 5% to about 95%.

153. The surface of any one of claims 148-152, wherein the coating layer comprises macropores.

154. The surface of any one of claims 148-153, wherein the coating layer comprises mesopores.

155. The electrode of any one of claims 148-154, wherein the coating layer comprises nanopores.

156. The surface of any one of claims 148-155, wherein the coating layer has a thickness from about 2 nm to about 100 μm.

157. The surface of any one of claims 148-156, wherein surface is a conductive substrate.

158. The surface of claim 157, wherein the conductive substrate comprises gold, silver, copper, platinum, aluminum, stainless steel, tungsten, indium tin oxide, titanium, lead, nickel, palladium, zirconium, niobium, tantalum, chromium, molybdenum, manganese, rhenium, ruthenium, rhodium, iridium, silicon, polyimide, parylene, benzocyclobutene, carbon, graphite, or any combination thereof.

159. The surface of claim 157 or 158, wherein the conductive substrate comprises a flexible substrate.

160. The electrode of claim 159, wherein the flexible substrate comprises polyethylene terephthalate, polyethylene naphathalate, polyimides, polymeric hydrocarbons, celluloses, plastics, polycarbonates, polystyrenes, or any combination thereof.

161. Use of an electrode of any one of claims 1-80 or sensor of any one of claims 81-84 for detecting a target analyte in a sample.

162. A method for detecting a target analyte in a sample, the method comprising:
a. contacting a sample suspected of comprising a target analyte with an electrode of any one of claims 1-161; and
b. detecting oxidation or reduction of the target analyte by the oxidoreductase present in or on the antifouling coating.

163. The method of any one of claim 162, wherein said detecting oxidation or reduction of the substrate comprises applying a voltage to the electrode.

164. The method of any one of claims 162-163, wherein said detecting oxidation or reduction of the substrate comprises measuring a current generated from electrode.

165. The method of any one of claims 162-164, wherein said detecting oxidation or reduction of the substrate comprises detecting oxidation or reduction of the redox mediator present in or on the antifouling coating.

166. The method of any one of claims 162-165, wherein the target analyte is a peptide, a polypeptide, a peptidomimetic, a nucleic acid, an oligosaccharide, a polysaccharide, an amino acid, nucleoside, a nucleotide, a carbohydrate, a lipid, a peptidoglycan, a cell, microbial matter, an antigen, a lipid, a steroid, a hormone, a lipopolysaccharide, an endotoxin, a therapeutic agent, a lipid-binding molecule, a cofactor, a small molecule, a toxin, a biological threat agent.

167. The method of any one of claims 162-166, wherein the target analyte is a protein, an antibody, an antigen binding fragment of an antibody, an antigen, a hormone, or a metabolite.

168. The method of any one of claims 162-167, wherein the target analyte is a tumour marker or a clinical chemistry target.

169. The method of any one of claims 162-168, wherein the sample is a biological sample.

170. The method of any one of claims 162-169, wherein the sample is a food, an ingredient for preparing a food, poultry, meat, fish, beverage, or dairy product.

171. The method of any one of claims 162-170, wherein the sample is a non-biological sample.

172. The method of any one of claims 162-171, wherein the sample is pre-processed prior to contacting with the electrode.

173. A kit comprising an electrode, sensor, composition or surface of any one of claims 1-172.
